SEQUENCE LISTING (92 sequences)

SEQ ID NO: 1 - DNA, 71 bp, Zea mays
  source 1..71: mol_type = genomic DNA
tcacagcgacgacaaggttagctagtactccccggycagtaatgtgttgaccaagagaaacaaagaatctc

SEQ ID NO: 2 - DNA, 64 bp, Zea mays
  source 1..64: mol_type = genomic DNA
atcttttctttagaccttagcaatcaagmctgatatttaaatccatcgtgaaaacatggaataa

SEQ ID NO: 3 - DNA, 45 bp, Zea mays
  source 1..45: mol_type = genomic DNA
gaccgcaagcaagatggtgtcctaagttagatgcckatggagtag

SEQ ID NO: 4 - DNA, 68 bp, Zea mays
  source 1..68: mol_type = genomic DNA
cagctgtagggtgaagcctatgacaaataaggrtcacatgggcctctcagagtctcagttgggctatg

SEQ ID NO: 5 - DNA, 71 bp, Zea mays
  source 1..71: mol_type = genomic DNA
acacctagagctccatttggtacatcaatgccctakcttagatgacataggtatgggcgtggctcgataga

SEQ ID NO: 6 - DNA, 42 bp, Zea mays
  source 1..42: mol_type = genomic DNA
tacactatttgtctcaattaccgaaacctaygcatgaagctt

SEQ ID NO: 7 - DNA, 71 bp, Zea mays
  source 1..71: mol_type = genomic DNA
atctagtgatataagcttatgagggctaaagatacwttaaagcagacaagaacttattaacatcctcgtcc

SEQ ID NO: 8 - DNA, 71 bp, Zea mays
  source 1..71: mol_type = genomic DNA
cttgcactgttcctatcacaaggttaatcagcgcakccagatcgctcgtgacgaagcgaaactgtttgtgt

SEQ ID NO: 9 - DNA, 43 bp, synthetic construct
  source 1..43: mol_type = other DNA
gaaggtcggagtcaacggattaggttagctagtactccccggt

SEQ ID NO: 10 - DNA, 48 bp, synthetic construct
  source 1..48: mol_type = other DNA
gaaggtcggagtcaacggattcttttctttagaccttagcaatcaagc

SEQ ID NO: 11 - DNA, 44 bp, synthetic construct
  source 1..44: mol_type = other DNA
gaaggtgaccaagttcatgctatggtgtcctaagttagatgccg

SEQ ID NO: 12 - DNA, 43 bp, synthetic construct
  source 1..43: mol_type = other DNA
gaaggtgaccaagttcatgctgactctgagaggcccatgtgat

SEQ ID NO: 13 - DNA, 45 bp, synthetic construct
  source 1..45: mol_type = other DNA
gaaggtcggagtcaacggattccatttggtacatcaatgccctag

SEQ ID NO: 14 - DNA, 43 bp, synthetic construct
  source 1..43: mol_type = other DNA
gaaggtgaccaagttcatgctccacctttggaagcttcatgcg

SEQ ID NO: 15 - DNA, 50 bp, synthetic construct
  source 1..50: mol_type = other DNA
gaaggtcggagtcaacggattgatataagcttatgagggctaaagatact

SEQ ID NO: 16 - DNA, 42 bp, synthetic construct
  source 1..42: mol_type = other DNA
gaaggtcggagtcaacggattcttcgtcacgagcgatctgga

SEQ ID NO: 17 - DNA, 42 bp, synthetic construct
  source 1..42: mol_type = other DNA
gaaggtgaccaagttcatgctggttagctagtactccccggc

SEQ ID NO: 18 - DNA, 50 bp, synthetic construct
  source 1..50: mol_type = other DNA
gaaggtgaccaagttcatgctatcttttctttagaccttagcaatcaaga

SEQ ID NO: 19 - DNA, 46 bp, synthetic construct
  source 1..46: mol_type = other DNA
gaaggtcggagtcaacggattagatggtgtcctaagttagatgcct

SEQ ID NO: 20 - DNA, 42 bp, synthetic construct
  source 1..42: mol_type = other DNA
gaaggtcggagtcaacggattactctgagaggcccatgtgac

SEQ ID NO: 21 - DNA, 45 bp, synthetic construct
  source 1..45: mol_type = other DNA
gaaggtgaccaagttcatgctccatttggtacatcaatgccctat

SEQ ID NO: 22 - DNA, 43 bp, synthetic construct
  source 1..43: mol_type = other DNA
gaaggtcggagtcaacggattccacctttggaagcttcatgca

SEQ ID NO: 23 - DNA, 50 bp, synthetic construct
  source 1..50: mol_type = other DNA
gaaggtgaccaagttcatgctgatataagcttatgagggctaaagataca

SEQ ID NO: 24 - DNA, 40 bp, synthetic construct
  source 1..40: mol_type = other DNA
gaaggtgaccaagttcatgcttcgtcacgagcgatctggc

SEQ ID NO: 25 - DNA, 28 bp, synthetic construct
  source 1..28: mol_type = other DNA
cacgagattctttgtttctcttggtcaa

SEQ ID NO: 26 - DNA, 30 bp, synthetic construct
  source 1..30: mol_type = other DNA
ccttttattccatgttttcacgatggattt

SEQ ID NO: 27 - DNA, 30 bp, synthetic construct
  source 1..30: mol_type = other DNA
cacaattatagcaccatcaaggacatattt

SEQ ID NO: 28 - DNA, 25 bp, synthetic construct
  source 1..25: mol_type = other DNA
gctgtagggtgaagcctatgacaaa

SEQ ID NO: 29 - DNA, 25 bp, synthetic construct
  source 1..25: mol_type = other DNA
ccacgcccatacctatgtcatctaa

SEQ ID NO: 30 - DNA, 30 bp, synthetic construct
  source 1..30: mol_type = other DNA
tacactatttgtctcaattaccgaaaccta

SEQ ID NO: 31 - DNA, 29 bp, synthetic construct
  source 1..29: mol_type = other DNA
tcgaaatcggacgaggatgttaataagtt

SEQ ID NO: 32 - DNA, 26 bp, synthetic construct
  source 1..26: mol_type = other DNA
gcttgcactgttcctatcacaaggtt

SEQ ID NO: 33 - DNA, 2383 bp, Zea mays
  source 1..2383: mol_type = genomic DNA
ctggaagcctgcttggcccaatcccccgctgcgcaaaagtgcaaaccgaaaccgaagtcagtggcagaaacggcccttatccacacacacaaaaaggtacgcgacacaagccacgctctccgcttgtggctaagctgctgcacgcgcggaccgacaaaaatccagcactagccaaagcacaggcgaagcagcagcagcgagaggccaccacaaacatggcgaccacggcgtttctacccccggccaagctgccgctgcaggggcgtcggttcgctagccccaaaacggcgggacgtatcagtgtcagggtccctccggcgcgagcgcagcaagagcagcaggtgaaggaggaggaggccgaggtggcgagcgtgccgccgccgcaggccgcggagaaggaggcgaggaagggggacgcgcagtcgctgccgcggcagcctttggcggagagcaagaacatgagccgcgagtacggcgggcagtggctgagcagcaccacgcggcacgtccgcatctacgctgcgtacatcgacccggagaccaacgcgttcgaccagacgcagatggacaagctgacgctcatgctcgacccccaggacgagttcgcctggaccgacgaggcctgccagatggtcttcaacgagttccaggacctcgtcgatcactacgaggtaacctctgtagttgctcccacagtcccactccaacagctccgatcaggggcggacccaggcccatggccgcctgggccacggcccggggcgcaggcccatttatttttttagaagatcctgggccacagcagagcgccgagcgcgtgacggattgacgcgcgcgaaccctagttcgcccgacttcactggctcgccgcgcgccgcgccccagtccgctctccgctggccgggtgggacgaggagtccgtcctcctcgccgcgatggtcgtcgaggacaccccagtctgcgagtcccgtcgcaagaggcgggcctccacctcctccgtcggcggcagcgccggatccagcaccaggtacggcggcctaccctcctaattctctatcctttttccttttcatgctcgggttcatcgtccctgacgtctgaatgccgccttctcgtaggaagaggaggccgcgtaggcagtctccggccaagatcctgccagtggttttcgcgatcgacgacgaaaagtcggacactgcggcaggtttgttcctcagatcgaatcgatttttttgggaaaattcctgctatgagtataatcttctttggggttctctatctggcgttcatttggaaatctcctagatggcaaatcggaagtgaaggccggcaaggaggaagagaagcctgtccttgtgggcgacaaggaggcgtccgattctggcgagaaggcagtggccgcggcaaacatgtcgtgcatggatcggctccgggaggagctctcttgcgcagtaagggtgcttcttttgtttttctttatatgatctgagctccgatagaatttatgatcgcttcggcccgggtcttggctaatttctgggtccgccactggctccgatagtagataaaaaaaatttgaattgaatctcttgttgtctgaaattcgtacggtcatttccccccttttcatctactttagagccactcctgccaaaaaaataaaacatacggtcttggcaaaagaagtgatcggtaagaggatagctgagctcgtttctgctttgaaacagggggctgagctgtcggagtacacacttcggttgatcgggtcagaccttgagcactacatccgcaaactgctgtacgacggggtgatcaagtacaacatgaggtccagagtcctcaatttcagcatgggcaagcctcgcatcaagttcaacagcaaccagataccagaaaagatatagaatgtacttatccaacaccctgaaaagaatggtttctctccattcgattaccgagcaagatgtatactctgcagtcccagatacgatccagagcattccagaattcatattacgcagatgcaaagccatacaccgattctgttggcaaaataagggtttgctcgagtttagataccccttctgtttctagggcactcgaattgggttacacgataccatagtttctgctaatagatacgtatcaccgaataccacatgaggtcagagagttgcacagagacgaccgcgctaacttaccaaacgactttctgaactggatggagtaatggctagtatgggtaaaccctctacaaacattccatgacaactcggtactttttaaattcatcgctccatggtctgaattggtcttctagctcaatgttctacagaagcataaagatacagtaagggctagtttgggaacctcaattttc

SEQ ID NO: 34 - DNA, 624 bp, Zea mays
  source 1..624: mol_type = genomic DNA
atggcgaccacggcgtttctacccccggccaagctgccgctgcaggggcgtcggttcgctagccccaaaacggcgggacgtatcagtgtcagggtccctccggcgcgagcgcagcaagagcagcaggtgaaggaggaggaggccgaggtggcgagcgtgccgccgccgcaggccgcggagaaggaggcgaggaagggggacgcgcagtcgctgccgcggcagcctttggcggagagcaagaacatgagccgcgagtacggcgggcagtggctgagcagcaccacgcggcacgtccgcatctacgctgcgtacatcgacccggagaccaacgcgttcgaccagacgcagatggacaagctgacgctcatgctcgacccccaggacgagttcgcctggaccgacgaggcctgccagatggtcttcaacgagttccaggacctcgtcgatcactacgagctgtcggagtacacacttcggttgatcgggtcagaccttgagcactacatccgcaaactgctgtacgacggggtgatcaagtacaacatgaggtccagagtcctcaatttcagcatgggcaagcctcgcatcaagttcaacagcaaccagataccagaaaagatatag

SEQ ID NO: 35 - protein, 207 aa, Zea mays
MATTAFLPPAKLPLQGRRFASPKTAGRISVRVPPARAQQEQQVKEEEAEVASVPPPQAAEKEARKGDAQSLPRQPLAESKNMSREYGGQWLSSTTRHVRIYAAYIDPETNAFDQTQMDKLTLMLDPQDEFAWTDEACQMVFNEFQDLVDHYELSEYTLRLIGSDLEHYIRKLLYDGVIKYNMRSRVLNFSMGKPRIKFNSNQIPEKI

SEQ ID NO: 36 - DNA, 1541 bp, Zea mays
  source 1..1541: mol_type = genomic DNA
ctggaagcctgcttggcccaatcccccgctgcgcaaaagtgcaaaccgaaaccgaagtcagtggcagaaacggcccttatccacacacacaaaaaggtacgcgacacaagccacgctctccgcttgtggctaagctgctgcacgcgcggaccgacaaaaatccagcactagccaaagcacaggcgaagcagcagcagcgagaggccaccacaaacatggcgaccacggcgtttctacccccggccaagctgccgctgcaggggcgtcggttcgctagccccaaaacggcgggacgtatcagtgtcagggtccctccggcgcgagcgcagcaagagcagcaggtgaaggaggaggaggccgaggtggcgagcgtgccgccgccgcaggccgcggagaaggaggcgaggaagggggacgcgcagtcgctgccgcggcagcctttggcggagagcaagaacatgagccgcgagtacggcgggcagtggctgagcagcaccacgcggcacgtccgcatctacgctgcgtacatcgacccggagaccaacgcgttcgaccagacgcagatggacaagctgacgctcatgctcgacccccaggacgagttcgcctggaccgacgaggcctgccagatggtcttcaacgagttccaggacctcgtcgatcactacgaggtaacctctgtagttgctcccacagtcccactccaacagctccgatagtagataaaaaaaatttgaattgaatctcttgttgtctgaaattcgtacggtcatttccccccttttcatctactttagagccactcctgccaaaaaaataaaacatacggtcttggcaaaagaagtgatcggtaagaggatagctgagctcgtttctgctttgaaacagggggctgagctgtcggagtacacacttcggttgatcgggtcagaccttgagcactacatccgcaaactgctgtacgacggggtgatcaagtacaacatgaggtccagagtcctcaatttcagcatgggcaagcctcgcatcaagttcaacagcaaccagataccagaaaagatatagaatgtacttatccaacaccctgaaaagaatggtttctctccattcgattaccgagcaagatgtatactctgcagtcccagatacgatccagagcattccagaattcatattacgcagatgcaaagccatacaccgattctgttggcaaaataagggtttgctcgagtttagataccccttctgtttctagggcactcgaattgggttacacgataccatagtttctgctaatagatacgtatcaccgaataccacatgaggtcagagagttgcacagagacgaccgcgctaacttaccaaacgactttctgaactggatggagtaatggctagtatgggtaaaccctctacaaacattccatgacaactcggtactttttaaattcatcgctccatggtctgaattggtcttctagctcaatgttctacagaagcataaagatacagtaagggctagtttgggaacctcaattttc

SEQ ID NO: 37 - DNA, 633 bp, Zea mays
  source 1..633: mol_type = other DNA
atggcgaccacggcgtttctacccccggccaagctgccgctgcaggggcgtcggttcgctagccccaaaacggcgggacgtatcagtgtcagggtccctccggcgcgagcgcagcaagagcagcaggtgaaggaggaggaggccgaggtggcgagcgtgccgccgccgcaggccgcggagaaggaggcgaggaagggggacgcgcagtcgctgccgcggcagcctttggcggagagcaagaacatgagccgcgagtacggcgggcagtggctgagcagcaccacgcggcacgtccgcatctacgctgcgtacatcgacccggagaccaacgcgttcgaccagacgcagatggacaagctgacgctcatgctcgacccccaggacgagttcgcctggaccgacgaggcctgccagatggtcttcaacgagttccaggacctcgtcgatcactacgagggggctgagctgtcggagtacacacttcggttgatcgggtcagaccttgagcactacatccgcaaactgctgtacgacggggtgatcaagtacaacatgaggtccagagtcctcaatttcagcatgggcaagcctcgcatcaagttcaacagcaaccagataccagaaaagatatag

SEQ ID NO: 38 - protein, 210 aa, Zea mays
MATTAFLPPAKLPLQGRRFASPKTAGRISVRVPPARAQQEQQVKEEEAEVASVPPPQAAEKEARKGDAQSLPRQPLAESKNMSREYGGQWLSSTTRHVRIYAAYIDPETNAFDQTQMDKLTLMLDPQDEFAWTDEACQMVFNEFQDLVDHYEGAELSEYTLRLIGSDLEHYIRKLLYDGVIKYNMRSRVLNFSMGKPRIKFNSNQIPEKI

SEQ ID NO: 39 - DNA, 1541 bp, Zea mays; identical to SEQ ID NO: 36
  source 1..1541: mol_type = genomic DNA

SEQ ID NO: 40 - DNA, 624 bp, Zea mays; identical to SEQ ID NO: 34
  source 1..624: mol_type = other DNA

SEQ ID NO: 41 - protein, 207 aa, Zea mays; identical to SEQ ID NO: 35

SEQ ID NO: 42 - DNA, 1380 bp, Zea mays
  source 1..1380: mol_type = genomic DNA
ccagcactagccaaagcacaggcgaagcagcagcagcgagaggccaccacaaacatggcgaccacggcgtttctacccccggccaagctgccgctgcaggggcgtcggttcgctagccccaaaacggcgggacgtatcagtgtcagggtccctccggcgcgagcgcagcaagagcagcaggtgaaggaggaggaggccgaggtggcgagcgtgccgccgccgcaggccgcggagaaggaggcgaggaagggggacgcgcagtcgctgccgcggcagcctttggcggagagcaagaacatgagccgcgagtacggcgggcagtggctgagcagcaccacgcggcacgtccgcatctacgctgcgtacatcgacccggagaccaacgcgttcgaccagacgcagatggacaagctgacgctcatgctcgacccccaggacgagttcgcctggaccgacgaggcctgccagatggtcttcaacgagttccaggacctcgtcgatcactacgaggtaacctctgtagttgctcccacagtcccactccaacagctccgatagtagataaaaaaaatttgaattgaatctcttgttgtctgaaattcgtacggtcatttccccccttttcatctactttagagccactcctgccaaaaaaataaaacatacggtcttggcaaaagaagtgatcggtaagaggatagctgagctcgtttctgctttgaaacagggggctgagctgtcggagtacacacttcggttgatcgggtcagaccttgagcactacatccgcaaactgctgtacgacggggtgatcaagtacaacatgaggtccagagtcctcaatttcagcatgggcaagcctcgcatcaagttcaacagcaaccagataccagaaaagatatagaatgtacttatccaacaccctgaaaagaatggtttctctccattcgattaccgagcaagatgtatactctgcagtcccagatacgatccagagcattccagaattcatattacgcagatgcaaagccatacaccgattctgttggcaaaataagggtttgctcgagtttagataccccttctgtttctagggcactcgaattgggttacacgataccatagtttctgctaatagatacgtatcaccgaataccacatgaggtcagagagttgcacagagacgaccgcgctaacttaccaaacgactttctgaactggatggagtaatggctagtatgggtaaaccctctacaaacattccatgacaactcggtactttttaaattcatcgctccatggtctgaattggtcttctagctcaatgttctacagaagcataaagatacagtaagggctagtttgggaacctcaattttc

SEQ ID NO: 43 - DNA, 633 bp, Zea mays; identical to SEQ ID NO: 37
  source 1..633: mol_type = other DNA

SEQ ID NO: 44 - protein, 210 aa, Zea mays; identical to SEQ ID NO: 38

SEQ ID NO: 45 - DNA, 1587 bp, Zea mays
  source 1..1587: mol_type = genomic DNA
ctggaagcctgcttggcccaatggtgcgctgtgcaaaagtgcaaaccgaaaccgaagtcagtggcagaaacggcccttatccacacacacaaaaaagtacgcgacacaagccacgctctccgcttgtggctaagctgctgcacgcgcggaacgacaaaaatccagcactagccaaagcacaggcgaagaagcaggcgagaggccaccacaaacatggcgaccacggcgtttctacccccggccaagctgccgctgcaggggcgtcggttcgctagccccaaaacggcgggacgtatcagcgtcagggtccctccggcgcgagcgcagcaagagcagcaggtgaaggaggaggaggccgaggtggcgagcgtgccgccgccacaggacgcggagaagcaggcgaggaagggggacgcgcagtccctgccgcggcagcctttggcggagagcaagaacatgagccgcgagtacggcgggcagtggctgagcagcaccacgcggcacgtccgcatctacgctgcgtacatcgacccggtgaccaacgcgttcgaccagacgcagatggacaagctgacgctcatgctcgacccccaggacgagttcgcctggaccgacgaggcctgccagatggtcttcaacgagttccaggacctcgtcgatcactacgaggtaacctctgtagttgctcccactccaacagctccgatagtagataaaaaaaatttgaattgaatctcttgttgtctgaaattcgtacggtcatttccccccttttcatctactttagagccactcctgccaaaaaaataaaacatacggtcttggcaaaagaagtgatcggtaagaggatagctgagctcgtttctgctttgaaacagggggctgagctgtcggagtacacacttcggttgatcgggtcagaccttgagcactacatccgcaaactgctgtacgacggggtgatcaagtacaacatgaggtccagagtcctcaatttcagcatgggcaagcctcgcatcaagttcaacagcaaccagataccagaaaagatatagaatgtacttatccaacgcccttaaaagaatggtttctctccattcgattaccgagcaagatgtatactctgcagtcccagatacgatccagagcattccagaattcatattacgcagatgcaaagccatacaccgattctgttggcagaattttgtcacccctgcacagtaataaccacataacgagagtctgggcaaaataagggtttgctcgagtttagataccccttctgtttctagggcactcgaattgggttacagtacacaataccatagtttctgctaatagatacgtatcaccgaataccacatgaggtcagagagttgcacagagacgaccgcgctaacttaccaaacgactttctgaactggatggagtaatggctagtatgggtaaaccctctacaaacattccatgacaactcggtactttttaaattcatcgctccatggtctgaattggtcttctagctcaatgttctacagaagcataaagatacagtaagggctagtttgggaacctcaattttct

SEQ ID NO: 46 - DNA, 633 bp, Zea mays
  source 1..633: mol_type = other DNA
atggcgaccacggcgtttctacccccggccaagctgccgctgcaggggcgtcggttcgctagccccaaaacggcgggacgtatcagcgtcagggtccctccggcgcgagcgcagcaagagcagcaggtgaaggaggaggaggccgaggtggcgagcgtgccgccgccacaggacgcggagaagcaggcgaggaagggggacgcgcagtccctgccgcggcagcctttggcggagagcaagaacatgagccgcgagtacggcgggcagtggctgagcagcaccacgcggcacgtccgcatctacgctgcgtacatcgacccggtgaccaacgcgttcgaccagacgcagatggacaagctgacgctcatgctcgacccccaggacgagttcgcctggaccgacgaggcctgccagatggtcttcaacgagttccaggacctcgtcgatcactacgagggggctgagctgtcggagtacacacttcggttgatcgggtcagaccttgagcactacatccgcaaactgctgtacgacggggtgatcaagtacaacatgaggtccagagtcctcaatttcagcatgggcaagcctcgcatcaagttcaacagcaaccagataccagaaaagatatag

SEQ ID NO: 47 - protein, 210 aa, Zea mays
MATTAFLPPAKLPLQGRRFASPKTAGRISVRVPPARAQQEQQVKEEEAEVASVPPPQDAEKQARKGDAQSLPRQPLAESKNMSREYGGQWLSSTTRHVRIYAAYIDPVTNAFDQTQMDKLTLMLDPQDEFAWTDEACQMVFNEFQDLVDHYEGAELSEYTLRLIGSDLEHYIRKLLYDGVIKYNMRSRVLNFSMGKPRIKFNSNQIPEKI

SEQ ID NO: 48 - DNA, 4185 bp, Zea mays
  source 1..4185: mol_type = genomic DNA
atggccgccgaggcgcccgtgcgagcccccgccacggcgcggtcctcgcgccggccggccgccgccgtcgtctccgcctcgcgcctcctcctcggccaccgccccttccttgcgccgcgcttcgctgcggggcgcgccgcggtggcaggccccgcggccggactacgcccgcgcccgcgcaggccgcgcctctcggtcgtcgtcatggccagcagcggtgagcgagcgctgtaatccatctgtggactctccattctcttattgggcgcagccgcgcaggcgttgctgtagtgcgccggcgcgtgagtgttctctgtgtttttcgaaatggccaagagcgtacctccgtagagctccggtggattgcagtatacgagagtgaaattaaggctgattgttagtgattgtgctgcatcgtgcttatccaaagggaccagtgtgcttcttgacgtgcttgtctctgccgtgtctataatacttgttttatgtttaggcccctatacttgttaatcgtctgtgctttacctgataaattacattaagttgttcgttactaaaatggtggaatatggtgttcagaatcgaagttatagcaatgatgaacttcatagctttctgatggacttttgtttctgtaggtgttattctaattaaatttggttatcacaaatccaggttagaagtaactgaaagcataggccttagaactattccaaaaatatttctaaacttcactacctatatatctatttaaagagccatcccatgaatatttttttgtgtgaaaaaattataagagaactaccttatttggaatttagagagccatttagcctctccattagcggagagcgagaagggtggttttaaaggggggaaaatatggagactggtgttgagcgtctattagagatgctcttaaggagacccaataatggaagttctgcgatttttgttcaaaaatggaagttctgcaaatgaaaaaattgggtccatggccttgttaagtgaatgtgccgccactacatgagtttgcatgatgtgaatttgctgcaagtacagaaatatcggtgcagagggtgtcaatatagaatataccatgcttgtcatgccgcttggtactagagatctctttttatgcaacttaaaatattggcacttgtcatatcaatgctacacctttctgccatatacatcaatgagactgaccaaatgaacctctgaaaattgcatgccagttgttttttaattacatttcttattgctaaatagtaaatacaacattcttgttggaaataatatatcactttgttggtttaacaagttagatattaacttgctttcttttgtatccagatcgccaagtgcctttacatgattaccgcaatattggtattatggcccatatagatgcaggaaagacaacaactactgagcgcattctgtattacactggaaggaactacaagattggcgaggttcatgagggaacagctacaatggactggatggaacaagaacaagagagaggaataaccattacatctgcagcaacgactgccttctggaacaaacacagaatcaacattattgatactcctggtcacgtcgacttcactcttgaggttgaacgtgctctcagggtgctggacggtgctatatgtctctttgacagtgttgctggggtagaaccacaatctgaaactgtgtggcgccaggcagataaatatggggttccaagaatatgtttcgtgaacaaaatggaccgccttggagctaacttctttagaactagagacatgatcgttgcaaatttgggtgccaaaccattggtgattcagttgccgattggttcggaggacaatttccaaggagttgttgatctagtcataatgaaagctattgtatggacaggggaggagctgggtgcaaaatttgaatacaaagacatacctgctgatctccaagagatggctcaagactatcgtgttcaaatgctggaaactattattgaattggatgatgaagttatggagaaatatcttgaaggaactgaaccagacgaggaaactgttaagaaactaatcagaaagggaacaatttctgccagctttgtaccagttttatgtggttcagcattcaaaaacaagggtgtccaaccattgcttgatgctgttgtcgattacttgccatctccacttgatctaccttcaatgaagggtactgacccagaagaccctgaaataatatttgaaaggcaaccaagtgacgatgagccattttctgggttagctttcaagatcatgactgatccatttgtggggtcactaacatttgttcgcatatactccgggaagctgatagctggttcatatgttctcaatgcaaataaagataagaaagaaagaattggaagacttcttgagatgcacgctaacagtaaggaagatataccagttgctgtgacaggtgacatagtagcgcttgctggtctgaaagacacaattactggtgaaacactctgtgacccagacaagcctgtagtgcttgaacgtatggaatttcctgatcctgtcattaaggttgctattgaacccaagaccaaagctgatgccgacaaaatggctactgggttaatcaagcttgctcaagaagacccgtcattccacttctctagagacgaggaaaccaaccagactgttattgaaggaatgggagaattgcatcttgatatcattgtagacagactaaagagggagttcaaggttagtattctgactagctgcatttatgcagttgcatgcgttgtatgtttctttaattctattctatatagaaatttattattcggtcattagctgttgtcaagtcagagttttgtacctgtcacattacctttacttttatgcaaacgatctgtgcaggttgaagcaaacgttggagctccacaagtcaactaccgtgaaagtatttccaaagttgcggaaatacaatatgtccacaaaaagcaatctggtggatctgggcagtttgcagacattattgtgcgttttgaacctttggaagctggaagtgggtatgagttcaagagtgaaatcaagggaggggcagtgcccaaagaatatgtaccaggggtgatgaaaggattggaagaaagcttacccaatggtgtcctcgccggttacccagttgtggacttccgggctgtgctggttgatggctcatatcacgatgtcgattcaagtgtcttagcattccaaattgcagccagaggagcattccgtgagggaatgaggaaagctggtccaaggcttctagagcctataatgaaagttgaagtgataactcctgaagagcatctgggtgatgttattggtgatttgaactctagaagaggacaggtcaacagcttcggggataagcctggtggactcaaggtatgaaacagtagatcatcccccttttgtacattagtttatcatatcctactatttgcaacgtgaccttaaacatttttcttgcatttctgttgatcacaattcacaggtggttgattcttttgtgccacttgctgagatgttccaatacgtcagcaccctccggggcatgaccaaggggcgagcgtcctacacgatgcagctcgccaagtttgacgtcgtcccacagcacatccagaaccagatctccgtggcaaaaacagaggaagctgctgcttagttttggttttccgtagatatatccatacagcaacaggagagcaggttgataggaaggacatcaaggtgtatgtagctttgtagtgtaaatctcttaaggcttgttcggatgattggaggggattgatatggattgagagagattttaatttactatggattgaaacttcctcaattcatatggattggactagaaccgaacatggtctaagggagcattatttgccattgattttgcacctgataataatggcctttgttgagttttcacaatgacaggacagttgaattatatgtttttggtttcctgctccctgttgagaactcagttctcaactttagtttggaaaacatagcacctgtaacaatataaattaatgcaagatggaaagaactttttttaaaaaatgttatattttaaacaaccatttcactctcatttatatacttgtgtcttggttatatagtt

SEQ ID NO: 49 - DNA, 2310 bp, Zea mays
  source 1..2310: mol_type = other DNA
atggccgccgaggcgcccgtgcgagcccccgccacggcgcggtcctcgcgccggccggccgccgccgtcgtctccgcctcgcgcctcctcctcggccaccgccccttccttgcgccgcgcttcgctgcggggcgcgccgcggtggcaggccccgcggccggactacgcccgcgcccgcgcaggccgcgcctctcggtcgtcgtcatggccagcagcgatcgccaagtgcctttacatgattaccgcaatattggtattatggcccatatagatgcaggaaagacaacaactactgagcgcattctgtattacactggaaggaactacaagattggcgaggttcatgagggaacagctacaatggactggatggaacaagaacaagagagaggaataaccattacatctgcagcaacgactgccttctggaacaaacacagaatcaacattattgatactcctggtcacgtcgacttcactcttgaggttgaacgtgctctcagggtgctggacggtgctatatgtctctttgacagtgttgctggggtagaaccacaatctgaaactgtgtggcgccaggcagataaatatggggttccaagaatatgtttcgtgaacaaaatggaccgccttggagctaacttctttagaactagagacatgatcgttgcaaatttgggtgccaaaccattggtgattcagttgccgattggttcggaggacaatttccaaggagttgttgatctagtcataatgaaagctattgtatggacaggggaggagctgggtgcaaaatttgaatacaaagacatacctgctgatctccaagagatggctcaagactatcgtgttcaaatgctggaaactattattgaattggatgatgaagttatggagaaatatcttgaaggaactgaaccagacgaggaaactgttaagaaactaatcagaaagggaacaatttctgccagctttgtaccagttttatgtggttcagcattcaaaaacaagggtgtccaaccattgcttgatgctgttgtcgattacttgccatctccacttgatctaccttcaatgaagggtactgacccagaagaccctgaaataatatttgaaaggcaaccaagtgacgatgagccattttctgggttagctttcaagatcatgactgatccatttgtggggtcactaacatttgttcgcatatactccgggaagctgatagctggttcatatgttctcaatgcaaataaagataagaaagaaagaattggaagacttcttgagatgcacgctaacagtaaggaagatataccagttgctgtgacaggtgacatagtagcgcttgctggtctgaaagacacaattactggtgaaacactctgtgacccagacaagcctgtagtgcttgaacgtatggaatttcctgatcctgtcattaaggttgctattgaacccaagaccaaagctgatgccgacaaaatggctactgggttaatcaagcttgctcaagaagacccgtcattccacttctctagagacgaggaaaccaaccagactgttattgaaggaatgggagaattgcatcttgatatcattgtagacagactaaagagggagttcaaggttgaagcaaacgttggagctccacaagtcaactaccgtgaaagtatttccaaagttgcggaaatacaatatgtccacaaaaagcaatctggtggatctgggcagtttgcagacattattgtgcgttttgaacctttggaagctggaagtgggtatgagttcaagagtgaaatcaagggaggggcagtgcccaaagaatatgtaccaggggtgatgaaaggattggaagaaagcttacccaatggtgtcctcgccggttacccagttgtggacttccgggctgtgctggttgatggctcatatcacgatgtcgattcaagtgtcttagcattccaaattgcagccagaggagcattccgtgagggaatgaggaaagctggtccaaggcttctagagcctataatgaaagttgaagtgataactcctgaagagcatctgggtgatgttattggtgatttgaactctagaagaggacaggtcaacagcttcggggataagcctggtggactcaaggtggttgattcttttgtgccacttgctgagatgttccaatacgtcagcaccctccggggcatgaccaaggggcgagcgtcctacacgatgcagctcgccaagtttgacgtcgtcccacagcacatccagaaccagatctccgtggcaaaaacagaggaagctgctgcttag

SEQ ID NO: 50 - protein, 769 aa, Zea mays
MAAEAPVRAPATARSSRRPAAAVVSASRLLLGHRPFLAPRFAAGRAAVAGPAAGLRPRPRRPRLSVVVMASSDRQVPLHDYRNIGIMAHIDAGKTTTTERILYYTGRNYKIGEVHEGTATMDWMEQEQERGITITSAATTAFWNKHRINIIDTPGHVDFTLEVERALRVLDGAICLFDSVAGVEPQSETVWRQADKYGVPRICFVNKMDRLGANFFRTRDMIVANLGAKPLVIQLPIGSEDNFQGVVDLVIMKAIVWTGEELGAKFEYKDIPADLQEMAQDYRVQMLETIIELDDEVMEKYLEGTEPDEETVKKLIRKGTISASFVPVLCGSAFKNKGVQPLLDAVVDYLPSPLDLPSMKGTDPEDPEIIFERQPSDDEPFSGLAFKIMTDPFVGSLTFVRIYSGKLIAGSYVLNANKDKKERIGRLLEMHANSKEDIPVAVTGDIVALAGLKDTITGETLCDPDKPVVLERMEFPDPVIKVAIEPKTKADADKMATGLIKLAQEDPSFHFSRDEETNQTVIEGMGELHLDIIVDRLKREFKVEANVGAPQVNYRESISKVAEIQYVHKKQSGGSGQFADIIVRFEPLEAGSGYEFKSEIKGGAVPKEYVPGVMKGLEESLPNGVLAGYPVVDFRAVLVDGSYHDVDSSVLAFQIAARGAFREGMRKAGPRLLEPIMKVEVITPEEHLGDVIGDLNSRRGQVNSFGDKPGGLKVVDSFVPLAEMFQYVSTLRGMTKGRASYTMQLAKFDVVPQHIQNQISVAKTEEAAA

SEQ ID NO: 51 - DNA, 4185 bp, Zea mays; identical to SEQ ID NO: 48
  source 1..4185: mol_type = genomic DNA

SEQ ID NO: 52 - DNA, 2310 bp, Zea mays; identical to SEQ ID NO: 49
  source 1..2310: mol_type = other DNA

SEQ ID NO: 53 - protein, 769 aa, Zea mays; identical to SEQ ID NO: 50

SEQ ID NO: 54 - DNA, 4171 bp, Zea mays
  source 1..4171: mol_type = genomic DNA
gaaaaaaacgctaatccataacgtgagacggggaggcgtcctcctagcagccgcgcgcgctcacattatcctcaggcaccatcgcaccattggccgccgctcacgcgcataaaacccacagtccgctcgcgacagcctcacggcgcaggcgagaaggcgaggagagggagctcgccggcaaaggccaccaagagcgaaaaggaaccacagaggcagtctccatggccgccgaggcgcccgtgcgagcccccgccacggcgcggtcctcgcgccggccggccgccgccgtcgtctccgcctcgcgcctcctcctcggccaccgccccttccttgcgccgcgcttcgctgcggggcgcgccgcggtggcaggccccgcggccggactacgcccgcgcccgcgcaggccgcgcctctcggtcgtcgtcatggccagcagcggtgagcgagcgctgtaatccttctgtggactctccattttcttattgggcgcagccgcgcaggcgttgctgtagtgcgccggcgcgtgagtgttttctgtgtttgtcgaaatggccaagagcgtacctccgtagagctccggtggattgcagtatacgagagtgaaattaaggctgattgttagtgattgtgctgcatcgtgcttatccaaagggaccagtgtgcttcttgacgtgcttgtctctgccgtgtctataatacttgtttaggcccctatacttgttaatcgtctgtgctttacctgataaattactttaagttgttcgttactaaaatggtggaatatggtgttcagaatcgaagttatagcaatgatgaacttcatagctttctgatggacttttgtttctgtaggtgttattctaattaaatttgattatcacaaatccaggttagaagtaactgaaagcataggccttataactattccaaaaatatttctaaacttcactacctatatatctatttaaagagccatcccatgaatatttttttgtgtgaaaaaattataagagaactaccatatttggaatttagagagccatttagcctctccattagtggaaagcgagaagggtggttttaaagggaggaaaatatggggactggtgttgagcgtctattagagatgctcttaaggagacccaataatggaagttctgcaatttttgttcaaaaatggaagttctgcaaatgaaaaaattgggtccatggccttgttcagtgaatgtgccgccgctacatgagtttgcatgatgtgaatttgctgcaagtacagaaatatcggtgcagagggtgtcaatatagaatataccatgcttggcatgccgcttggtactagatatctattggcacttgtcatatcaatgctaaacctttctgccatatacatcaatgagactgaccaaatgaacctctgaaaattgcatgccagttgttttttaattacatttcttattgctaaatacaccattcttatatcactttgttgatttaacaagttagatattaacttgctttcttttgtatccagatcgccaagtgcctttacatgattaccgcaatattggtattatggcccatatagatgcaggaaagacaacaactactgagcgcattctgtattacactggaaggaactacaagattggtgaggttcatgagggaacagctacaatggattggatggaacaagaacaagagagaggaataaccattacatctgcagcaacgactgccttctggaacaaacacagaatcaacattattgatactcctggtcacgtcgacttcactcttgaggttgaacgtgctctcagggtgctggacggtgctatatgtctctttgacagtgttgctggggtagaaccacaatctgaaactgtgtggcgccaggcagataaatatggggttccaagaatatgtttcgtgaacaaaatggaccgccttggagctaacttctttagaactagagacatgatcgttgcaaatttgggtgccaaaccattggtgattcagttgccgattggttcggaggacaatttccaaggagttgttgatctagtaagaatgaaagctattgtatggacaggggaggagctgggtgcaaaatttgaatacaaagacatacctgctgatctccaagagatggctcaagactatcgtgttcaaatgctggaaactattattgaattggatgatgaagttatggagaaatatcttgaaggaactgaaccagacgaggaaactgttaagaaactaatcagaaagggagcaatttctgccagctttgtcccagttttatgtggttcggcattcaaaaacaagggtgtccaaccattgcttgatgctgttgtcgattacttgccatctccacttgatctaccttcaatgaagggtactgacccagaagaccctgaaataatatttgaaaggcaaccaagtgacgatgaaccattttctgggttagctttcaagatcatgactgatccattcgtggggtcactaacatttgttcgcatatactccgggaagctgatagctggttcctatgttctcaatgcaaataaagataagaaagaaagaattggaagacttcttgagatgcacgcaaacagtaaggaagatataccagttgctgtgacaggtgacatagtagcgcttgctggtctgaaagacacaattactggtgaaacactctgtgacccagacaagcctgtagtgcttgaacgtatggaatttcctgatcctgtcattaaggttgctattgaacccaagaccaaagctgatgccgacaaaatggctactgggttaatcaagcttgctcaagaagacccgtcattccacttctctagagacgaggaaaccaaccagactgttattgaaggaatgggagaactgcatcttgatatcattgtagacagactaaagagggagttcaaggttagtattctgactagctgcatttatgcagttgcatgcgctgtatgtttctttaattctattctatatagaaatttattattcggtcattagctgttgtcgagtcagagttttgtacctgtcacattacctttacttcttttatgcaaacgatctgtgcaggttgaagcaaacgttggagctccacaagtcaactaccgtgaaagtatttccaaagttgcggaaatacaatatgtccacaaaaagcaatctggtggatctgggcagtttgcagacattattgtgcgttttgaacctttggaagctggaagtgggtatgagttcaagagtgaaatcaagggaggggcagtgcccaaagaatatgtaccaggggtgatgaaaggattggaagaaagcttacccaatggtgtcctagccggttacccagttgtggacttccgggctgtgctggttgatggctcatatcacgatgtcgattcaagtgtcttagcattccaaattgcagccagaggagcattccgtgagggaatgaggaaagctggtccaaggcttctagagcctataatgaaagttgaagtgataactcctgaagagcatctgggtgatgttattggtgatttgaactctagaagaggacaggtcaacagcttcggggataagcctggtggactcaaggtatgaaacagtagatcatcccccttttgtacattagtttatcatatcctactatttgcaacgtgaccttaaacatttttcttgcatttctgttgatcacaattcacaggtggttgattcttttgtgccacttgctgagatgttccaatacgtcagcaccctccggggcatgaccaaggggcgagcgtcctacacgatgcagctcgccaagtttgacgtcgtcccacagcacatccagaaccagatctccgtggcaaaaacagaggaagctgctgcttagttttggttttccgtagatatatccatacagcaacaggagagcaggttgataggaaggacatcaaggtgtatgtagctttgtagtgtaaatctcttaaggcttgttcggatgattggaggggattgatatggattgagagagattttaatttactatggattgaaacttcctcaattcatatggattggactagaaccgaacatggtctaagggagcattatttgccattgattttgcacctgataataatggcctttgttgagttttcacaatgac

SEQ ID NO: 55 - DNA, 2310 bp, Zea mays
  source 1..2310: mol_type = other DNA
atggccgccgaggcgcccgtgcgagcccccgccacggcgcggtcctcgcgccggccggccgccgccgtcgtctccgcctcgcgcctcctcctcggccaccgccccttccttgcgccgcgcttcgctgcggggcgcgccgcggtggcaggccccgcggccggactacgcccgcgcccgcgcaggccgcgcctctcggtcgtcgtcatggccagcagcgatcgccaagtgcctttacatgattaccgcaatattggtattatggcccatatagatgcaggaaagacaacaactactgagcgcattctgtattacactggaaggaactacaagattggtgaggttcatgagggaacagctacaatggattggatggaacaagaacaagagagaggaataaccattacatctgcagcaacgactgccttctggaacaaacacagaatcaacattattgatactcctggtcacgtcgacttcactcttgaggttgaacgtgctctcagggtgctggacggtgctatatgtctctttgacagtgttgctggggtagaaccacaatctgaaactgtgtggcgccaggcagataaatatggggttccaagaatatgtttcgtgaacaaaatggaccgccttggagctaacttctttagaactagagacatgatcgttgcaaatttgggtgccaaaccattggtgattcagttgccgattggttcggaggacaatttccaaggagttgttgatctagtaagaatgaaagctattgtatggacaggggaggagctgggtgcaaaatttgaatacaaagacatacctgctgatctccaagagatggctcaagactatcgtgttcaaatgctggaaactattattgaattggatgatgaagttatggagaaatatcttgaaggaactgaaccagacgaggaaactgttaagaaactaatcagaaagggagcaatttctgccagctttgtcccagttttatgtggttcggcattcaaaaacaagggtgtccaaccattgcttgatgctgttgtcgattacttgccatctccacttgatctaccttcaatgaagggtactgacccagaagaccctgaaataatatttgaaaggcaaccaagtgacgatgaaccattttctgggttagctttcaagatcatgactgatccattcgtggggtcactaacatttgttcgcatatactccgggaagctgatagctggttcctatgttctcaatgcaaataaagataagaaagaaagaattggaagacttcttgagatgcacgcaaacagtaaggaagatataccagttgctgtgacaggtgacatagtagcgcttgctggtctgaaagacacaattactggtgaaacactctgtgacccagacaagcctgtagtgcttgaacgtatggaatttcctgatcctgtcattaaggttgctattgaacccaagaccaaagctgatgccgacaaaatggctactgggttaatcaagcttgctcaagaagacccgtcattccacttctctagagacgaggaaaccaaccagactgttattgaaggaatgggagaactgcatcttgatatcattgtagacagactaaagagggagttcaaggttgaagcaaacgttggagctccacaagtcaactaccgtgaaagtatttccaaagttgcggaaatacaatatgtccacaaaaagcaatctggtggatctgggcagtttgcagacattattgtgcgttttgaacctttggaagctggaagtgggtatgagttcaagagtgaaatcaagggaggggcagtgcccaaagaatatgtaccaggggtgatgaaaggattggaagaaagcttacccaatggtgtcctagccggttacccagttgtggacttccgggctgtgctggttgatggctcatatcacgatgtcgattcaagtgtcttagcattccaaattgcagccagaggagcattccgtgagggaatgaggaaagctggtccaaggcttctagagcctataatgaaagttgaagtgataactcctgaagagcatctgggtgatgttattggtgatttgaactctagaagaggacaggtcaacagcttcggggataagcctggtggactcaaggtggttgattcttttgtgccacttgctgagatgttccaatacgtcagcaccctccggggcatgaccaaggggcgagcgtcctacacgatgcagctcgccaagtttgacgtcgtcccacagcacatccagaaccagatctccgtggcaaaaacagaggaagctgctgcttag

SEQ ID NO: 56 - protein, 769 aa, Zea mays
MAAEAPVRAPATARSSRRPAAAVVSASRLLLGHRPFLAPRFAAGRAAVAGPAAGLRPRPRRPRLSVVVMASSDRQVPLHDYRNIGIMAHIDAGKTTTTERILYYTGRNYKIGEVHEGTATMDWMEQEQERGITITSAATTAFWNKHRINIIDTPGHVDFTLEVERALRVLDGAICLFDSVAGVEPQSETVWRQADKYGVPRICFVNKMDRLGANFFRTRDMIVANLGAKPLVIQLPIGSEDNFQGVVDLVRMKAIVWTGEELGAKFEYKDIPADLQEMAQDYRVQMLETIIELDDEVMEKYLEGTEPDEETVKKLIRKGAISASFVPVLCGSAFKNKGVQPLLDAVVDYLPSPLDLPSMKGTDPEDPEIIFERQPSDDEPFSGLAFKIMTDPFVGSLTFVRIYSGKLIAGSYVLNANKDKKERIGRLLEMHANSKEDIPVAVTGDIVALAGLKDTITGETLCDPDKPVVLERMEFPDPVIKVAIEPKTKADADKMATGLIKLAQEDPSFHFSRDEETNQTVIEGMGELHLDIIVDRLKREFKVEANVGAPQVNYRESISKVAEIQYVHKKQSGGSGQFADIIVRFEPLEAGSGYEFKSEIKGGAVPKEYVPGVMKGLEESLPNGVLAGYPVVDFRAVLVDGSYHDVDSSVLAFQIAARGAFREGMRKAGPRLLEPIMKVEVITPEEHLGDVIGDLNSRRGQVNSFGDKPGGLKVVDSFVPLAEMFQYVSTLRGMTKGRASYTMQLAKFDVVPQHIQNQISVAKTEEAAA

SEQ ID NO: 57 - DNA, 3129 bp, Zea mays
  source 1..3129: mol_type = genomic DNA
gcgtctgtccggcgggttctcgttttctcctgttgtccggaacattccagaatcgtggagaccggtcgggactcgggactagaacccgtgccttctctctccactctttatacttgcccctcctcgacggttcgccaaaaggttcgagacgcgcaccgccagccgatacgaatccgacgagctcgatcgctccacttccccgatcgcacgccaatcagcaccagccaccgtggcgatgacggaggaggcggaggcgaaggcgaagggcaacgcggcgttcgcggccggccgtttcgaggaggctgtccagcacttcagcgacgccatcgcgctcgcgcccgacaaccacgtcctctactccaaccgctcggcggcgtacgcgtcgctctggcgctacgcggaggcgctagatgacgccaagcggaccgtggcgctgaagccggactgggccaagggctactcccgcctcggcgccgcgcaccttggcctcggtgacgcgcccaaggccgtcgaggcctacgagaaagggcttgcgctcgagccgtccaatgaggcgctcaaatccggcctcgcacaggcgcgccagtccgcatcggcgccgagacgtccggtgggctccggggctgacgccgttggcaagatgttccagggccctgagctatggagcaaggtcgcggccgatcccaccacgcgtggctacctcgaccagccggacttcgtgcagatgctgcgggaggtgcagcgaaaccccggcagactgaacagctacctttctgaccaacgaatggttcaggtgctcaccctcatgctgaacatcaagttccagaaccagagcagtggggcgcctgacccggcagcagcacaatcgaacccgccgccaccaaagcagcagccagaggcgaaggcaagagaaccagagccggacccggagcccgagcctatggaggtgacagaggaggataaggagcagaaggagaggaaagcgtcggcgcagaaggagaaggaagcaggcaacgccgcttacaaaaggaaggattttgataccgcgatccagcactacacagaagccatggagctcgatgacgaggatatctcctacatcaccaatcgtgctgctgtttaccttgagatgggaaaggtaaggactcgtaggtccctagttctaaatcacaagttatttacatatatatgtagtctttagttaaagaaaaaaaacgtaaaatgtgggttgtctgaattttgtcaatgtgatcagctaccgagaactttttacaacgtactgctaacagcttttagatataaaattggctaagtattggtctctccttggagtaatggggtcaattgatattccagttctacttgaatttgtcgagaactcgagttgcattggcctcctttggatcaaaggattggaaaaatgtaggaataggaaaaatacaggaatatgataggaatgccagggtaaaacacaggattgtagaagaggatttttgtgagaatggtcctttggataggctcgaggaaaaacacaggatttttaaagacaatagattcaaatgaatgtcaaattgtatttactaacatcatctgcaattgattatccatgtattagatgacttaggtcccatctagcttgtggttggcctcgatcctatgcgacaaggaagaaaaaatgaggttcaagtggatgttttaaatcctccaaatttcccatgaaactgtcaccttggaaattttcctaggcttttcccatgccctaatcctctgttccaaagggtgctatagtaccaattccaacaggaatgagaatcccaatgttttcgcgtgaaattcctttgatccaaaggagccctaaaaggaagttttgtggtacgagttggtgattgtgacagtgagcaaaatacattagtgttccacagtcatgaaattctgatacaggtagggtggtaatgggctctagattttacaatataaaatttaaggattgaattggattaggatcaggttctattcctattcattttttgaactaaaattagttaagggctctaacttattgtgaagaaatatttggatcgtgttccattctcacccctagatacatgccacttgggagttccctagtcctgaaattctgatatggacaacttggagcttgatgagtagacgcaagccagaacgacattgttgttgaagtgattccttgtatttttttcttactccaagtcgtgaatgaattcacttctattgtcggcttatgcttaggcaatgcaaaaggcttatctggtgtccttcgaaactaatatttttttctttgaattccagtatgatgaatgcattaaggactgtgataaggctgtcgagagaggaagggaactgcatgctgatttcaagatgatctcaagggcacttaccagaaaaggaactgctcttgccaaacttgctaaatcatcgcaagactatgatgctgccattgagattttccagaaggcgttaactgagcaccggaacccagatacactgaaaaaactaaatgatgcagaacgagcaaagaaggagctggagcaacaagagtactatgacccaagaattgctgacgaggagcgagaaaaaggtacgccacatgacttctttctgctaccagtcatcacttgatcattattgataaagtgagttctgttgcctgtagtttgtttactgtcaagaattgcatgtggtgttggcgttgaaccatttattatttctgtgcaggtaatgagttctttaagcagcagaaatatccagaagcagtgaaacattacaccgaagctctcaggagaaaccccaaggaccccagggtaatttgctgtacatcctctctcacttgcataatctgtagtctgcctagtatcactgtgcaacctttaatctgctgggtaaaattatttgatagtattgttggaatgtgaaccctaaccctaaacaggggtgagaaggtcattacacggggaagaccctaaaccctaaacgggtattctaacaccccccgcagtcacaactctatcctgtacagatgttgagactggatcgaaagtctaaaaatacactcgacggtaaccccttggtgaagatgtcggcgaactgtag

SEQ ID NO: 58 - DNA, 1305 bp, Zea mays
  source 1..1305: mol_type = other DNA
atgacggaggaggcggaggcgaaggcgaagggcaacgcggcgttcgcggccggccgtttcgaggaggctgtccagcacttcagcgacgccatcgcgctcgcgcccgacaaccacgtcctctactccaaccgctcggcggcgtacgcgtcgctctggcgctacgcggaggcgctagatgacgccaagcggaccgtggcgctgaagccggactgggccaagggctactcccgcctcggcgccgcgcaccttggcctcggtgacgcgcccaaggccgtcgaggcctacgagaaagggcttgcgctcgagccgtccaatgaggcgctcaaatccggcctcgcacaggcgcgccagtccgcatcggcgccgagacgtccggtgggctccggggctgacgccgttggcaagatgttccagggccctgagctatggagcaaggtcgcggccgatcccaccacgcgtggctacctcgaccagccggacttcgtgcagatgctgcgggaggtgcagcgaaaccccggcagactgaacagctacctttctgaccaacgaatggttcaggtgctcaccctcatgctgaacatcaagttccagaaccagagcagtggggcgcctgacccggcagcagcacaatcgaacccgccgccaccaaagcagcagccagaggcgaaggcaagagaaccagagccggacccggagcccgagcctatggaggtgacagaggaggataaggagcagaaggagaggaaagcgtcggcgcagaaggagaaggaagcaggcaacgccgcttacaaaaggaaggattttgataccgcgatccagcactacacagaagccatggagctcgatgacgaggatatctcctacatcaccaatcgtgctgctgtttaccttgagatgggaaagtatgatgaatgcattaaggactgtgataaggctgtcgagagaggaagggaactgcatgctgatttcaagatgatctcaagggcacttaccagaaaaggaactgctcttgccaaacttgctaaatcatcgcaagactatgatgctgccattgagattttccagaaggcgttaactgagcaccggaacccagatacactgaaaaaactaaatgatgcagaacgagcaaagaaggagctggagcaacaagagtactatgacccaagaattgctgacgaggagcgagaaaaaggtaatgagttctttaagcagcagaaatatccagaagcagtgaaacattacaccgaagctctcaggagaaaccccaaggaccccagggtaatttgctatgtcggcgaactgtag

SEQ ID NO: 59 - protein, 434 aa, Zea mays
MTEEAEAKAKGNAAFAAGRFEEAVQHFSDAIALAPDNHVLYSNRSAAYASLWRYAEALDDAKRTVALKPDWAKGYSRLGAAHLGLGDAPKAVEAYEKGLALEPSNEALKSGLAQARQSASAPRRPVGSGADAVGKMFQGPELWSKVAADPTTRGYLDQPDFVQMLREVQRNPGRLNSYLSDQRMVQVLTLMLNIKFQNQSSGAPDPAAAQSNPPPPKQQPEAKAREPEPDPEPEPMEVTEEDKEQKERKASAQKEKEAGNAAYKRKDFDTAIQHYTEAMELDDEDISYITNRAAVYLEMGKYDECIKDCDKAVERGRELHADFKMISRALTRKGTALAKLAKSSQDYDAAIEIFQKALTEHRNPDTLKKLNDAERAKKELEQQEYYDPRIADEEREKGNEFFKQQKYPEAVKHYTEALRRNPKDPRVICYVGEL

SEQ ID NO: 60 - DNA, 3129 bp, Zea mays; identical to SEQ ID NO: 57
  source 1..3129: mol_type = genomic DNA

SEQ ID NO: 61 - DNA, 1305 bp, Zea mays; identical to SEQ ID NO: 58
  source 1..1305: mol_type = other DNA

SEQ ID NO: 62 - protein, 434 aa, Zea mays; identical to SEQ ID NO: 59

SEQ ID NO: 63 - DNA, 9406 bp, Zea mays
  source 1..9406: mol_type = genomic DNA
gcgtctgtccggcgggttctcgttttctcctgttgtccggaacattccagaatcgtggagaccggtcgggactcgggactagaacccgtgccttctctctccactctttatacttgcccctcctcgacggttcgccaaaaggttcgagacgcgcaccgccagccgatacgaatccgacgagctcgatcgctccacttccccgatcgcacgccaatcagcaccagccaccgtggcgatgacggaggaggcggaggcgaaggcgaagggcaacgcggcgttcgcggccggccgtttcgaggaggctgtccagcacttcagcgacgccatcgcgctcgcgcccgacaaccacgtcctctactccaaccgctcggcggcgtacgcgtcgctctggcgctacgcggaggcgctagatgacgccaagcggaccgtggcgctgaagccggactgggccaagggctactcccgcctcggcgccgcgcaccttggcctcggtgacgcgcccaaggccgtcgaggcctacgagaaagggcttgcgctcgagccgtccaatgaggcgctcaaatccggcctcgcacaggcgcgccagtccgcatcggcgccgagacgtccggtgggctccggggctgacgccgttggcaagatgttccagggccctgagctatggagcaaggtcgcggccgatcccaccacgcgtggctacctcgaccagccggacttcgtgcagatgctgcgggaggtgcagcgaaaccccggcagactgaacagctacctttctgaccaacgaatggttcaggtgctcaccctcatgctgaacatcaagttccagaaccagagcagtggggcgcctgacccggcagcagcacaatcgaacccgccgccaccaaagcagcagccagaggcgaaggcaagagaaccagagccggacccggagcccgagcctatggaggtgacagaggaggataaggagcagaaggagaggaaagcgtcggcgcagaaggagaaggaagcaggcaacgccgcttacaaaaggaaggattttgataccgcgatccagcactacacagaagccatggagctcgatgacgaggatatctcctacatcaccaatcgtgctgctgtttaccttgagatgggaaaggtaaggactcgtaggtccctagttctaaatcacaagttatttacatatatatgtagtctttagttaaagaaaaaaaacgtaaaatgtgggttgtctgaattttgtcaatgtgatcagctaccgagaactttttacaacgtactgctaacagcttttagatataaaattggctaagtattggtctctccttggagtaatggggtcaattgatattccagttctacttgaatttgtcgagaactcgagttgcattggcctcctttggatcaaaggattggaaaaatgtaggaataggaaaaatacaggaatatgataggaatgccagggtaaaacacaggattgtagaagaggatttttgtgagaatggtcctttggataggctcgaggaaaaacacaggatttttaaagacaatagattcaaatgaatgtcaaattgtatttactaacatcatctgcaattgattatccatgtattagatgacttaggtcccatctagcttgtggttggcctcgatcctatgcgacaaggaagaaaaaatgaggttcaagtggatgttttaaatcctccaaatttcccatgaaactgtcaccttggaaattttcctaggcttttcccatgccctaatcctctgttccaaagggtgctatagtaccaattccaacaggaatgagaatcccaatgttttcgcgtgaaattcctttgatccaaaggagccctaaaaggaagttttgtggtacgagttggtgattgtgacagtgagcaaaatacattagtgttccacagtcatgaaattctgatacaggtagggtggtaatgggctctagattttacaatataaaatttaaggattgaattggattaggatcaggttctattcctattcattttttgaactaaaattagttaagggctctaacttattgtgaagaaatatttggatcgtgttccattctcacccctagatacatgccacttgggagttccctagtcctgaaattctgatatggacaacttggagcttgatgagtagacgcaagccagaacgacattgttgttgaagtgattccttgtatttttttcttactccaagtcgtgaatgaattcacttctattgtcggcttatgcttaggcaatgcaaaaggcttatctggtgtccttcgaaactaatatttttttctttgaattccagtatgatgaatgcattaaggactgtgataaggctgtcgagagaggaagggaactgcatgctgatttcaagatgatctcaagggcacttaccagaaaaggaactgctcttgccaaacttgctaaatcatcgcaagactatgatgctgccattgagattttccagaaggcgttaactgagcaccggaacccagatacactgaaaaaactaaatgatgcagaacgagcaaagaaggagctggagcaacaagagtactatgacccaagaattgctgacgaggagcgagaaaaaggtacgccacatgacttctttctgctaccagtcatcacttgatcattattgataaagttagttctgttgcctgtagtttgtttactgtcaagaattgcatgtggtgttggcgttgaaccatttattatttctgtgcaggtaatgagttctttaagcagcagaaatatccagaagcagtgaaacattacaccgaagctctcaggagaaaccccaaggaccccagggtaatttgctgtacatcctctctcacttgcataatctgtagtctgcctagtatcactgtgcaacctttaatctgctgggtaaaattatttgatagtattgttggaatgtgaaccctaaccctaaacaggggtgagaaggtcattacacggggaagaccctaaaccctaaacgggtattctaacaccccccgcagtcacaactctatcctgtacagatgttgagactggatcgaaagtctaaaaatacactcgacggtaaccccttggtgaagatgtcggcgaactgtagcgcggtggggacgctgagaacccgaacgtcaccggcagcgacacgctcgcggacgaagtgcaggtcgatctccacatgcttcgtgcgctaatgctgcacggggttggtggagaggtagaccgcgctgacgttgtcgcagtagacgagggtggcgcgctggaggggactgtggagctcgtggaggagctggcgcagccaggaggcctctgccacgccgttggccacgacacgttactcggccttagcgctggagcgagagacgacgggctgacgcttggcggcccaagatacgaggttggcgcccaggaacacggtgtaaccggaggtgtaccggcgcgtgtcgggacagccagcccagtcagcgtcggtgtagaccacgagctccgacgttggggatggtcggagaaggaggccgtagtcgagggagccgcagaggtagcgcagaatccgcttgagcgcagtgagatggggctcccgcggagtgtgcatatgcaggcacacctgctggacggcgtatgcgatgtcgggcctggagaaggtgaggtactggagcgcgtcggtcaggctccgataggacatcgcgtcggcgactggaggcccgtcgtcctcagagagcttcgcctgagtgtcgacaggcgtggagcagggcttgcagtcagacatgccagcccgctccaggatgtcgatggcgtactggtgctggtggaggaagagaccctgaggccggcgctcggcggtgatgccgaggaagtggtgtagttgccctaggtccttcatcgcgaactcccgctgaagggcgaggatcgtgcggtgtagaaggtcggcggtggatgccgtgagcacaatgtcgtcgacgtagagcaggagatagacaatgtcgtcgccgcgccggtagatgaacagggacgtgtctgacttggcctcaacgaagccgatggaggccaggtaggaggcgaagcgactgtaccatgcccgtggcgcctgcttgaggccgtacagggatcggttcagccggcagaccagatccggacggtcagcgtcgacgaagccggtgggctggctgcagtagacagcctccgttagagtgccatggaggaaggcattcttgacatcgagctgatggatcgcccagtcgtgggagagggcgagggagaggacggcgcgaacagtggcgaatttgacgacggggctgaaggtctcgtcgtagtccactccggggcgctgtgtgaagccccgaaggacccaacgggacttgtagcggtcgagggagccatccgaggtcagcttgtggcgaaatagccacttgccggtgaccacgttggtgcctggtggacacggcactaggtcccaggtgtggttggccaatagggccgcgtactcctccatagcacgacgccaatgtgggtcggcgagggcagtgcgaacggaggagggcaccggggaagcgtcgggtggagtgctggacgtatcagcagccaggatcagccggtcgacggggcgaagaacgccagcggcgcgtcgagtcaccatcgggtggacgtgtccggggtcgcggtggatggcgaccgggtggtatacggacggctcggagcggaccgccggaacgtcgagagcggcgggtgtggccggcatgcggcggtgatagacgacggcggggtcggcgaagcgtgtcgcgctcgacgacgggcccgagtcggggggcgcggaggtagtggcttggctgcggcggtggtagacgagcgtggggttggcgaagcgagccggggtcgacggggccgcgcgtggcgcaggcaggatcgacggggccgcgcgtggcgcaggcaggatcgacggggccgcgcatggcgcaggcatgatcgacgtggccgcgcgtggcgcaggcggggtcgacggggccgcgcgtggcgcggaaatgggcgctagctgtggcgtcgtggctgcacgaggagcaggtaacggcgcaagacggggcgccgggggtgggggggaaccgggtcagactcgaggagggagtcaagagcggtgggtggggtggagcctgcaaggggaaacacatcttcgtcgaagacgacgtgacgagagatgatgatgcggtgggaggtgaggtccaagcaccgataccccttgtggtcaggggagtagccgaggaagaggcaacgagtggagcgaggagaaagcttgttaggggcggtagcggaaatgttagggtagcagtcacagccgaacacgcgcaggtggtcgtaggaaggggttgtgccgtacagggcaaagtgaggtgtagggtggctcaccgccttcgagggaagacggttgaggagatgcatggcggtatgaagggcctctgcccagtaggtggcagggagagacgcctgaaagagaaggcagcgaatcatgttggtgtggtgcgaatcatgcgctcggcccggccgttctgagcagaggtgtagggacacgagagacgcaactggacgccatgagtgaggaagaaagagcgggaggcgttgttgtcgaactcgtggccattgtcgcactgcagggcacagaccgggcaccggaactgggtggatacccaggcgaagaagtgagtgagggtggtaaacgtgtcggacttcagccgaagggggaaagtccaaaggaagtgggagtagtcatccagaatgaccaggtagtatttgtagccggagaggctaaggacaggagatgtccagagatcacagtggacaagatcaaaggcctgaacaaccctggacgtggaagtggaaaaaggaagacgagagtgacggccgagctgacaagcatgacagaggcgttcagaagagccctgagtatatgatatgtcggagtggccggaaagctgggatatgacgtcaggtccagggtgctcgaggcgacggtgccaaatggcggaggaggtggtggtagtagcaagagcatgtgatgtggtggtagtagtaagtacaggagatgaggctactctggtgtggggagtggagggcaagtgaagagaatagagggggccgaagctgtcacggcgagcgagcacaacatgtgtgggaaggtggcgtatggtgagaccccaggggtcgaactccatagagcactggttgtcactagtgaagcgacgaaccgagaggaggggatgagtcagtccgggagcaacaaggacgtcgttaaggcagaatggtcctggaagaaccgatgtacccactgaggtgaccgggagggtggaaccgttgccgacgacgatggaggtaggatgtgtgaggtggggtggatgggagtggagtaacgaattagcggtgggggtagtgtggaaggaggccctggagtcaaccacccaatcggtggtggttcggggaggtggaagtggcgaaggtacggtgtgagcggagagggccaaagaaggtgcaccgacagaggcactaggaagggagggagatgacacgggctcaatcgctagggaggcgatcgcagcacgtgggaagacaagctgtaagggcccgcctgagggtggagctcgacgcagtcccgaagaatagggttccagattggatcgaaggacacgaacatgccccggatgagctggccatcgtggaggaggacacacatagtcacgagagcggcaggcgaggtaaagctcatggcgaccggtgcaggagggtcgaggtactgctggggccggggcgcggtcagccggcgcagggagacgcggactgggcagccgggcgtggaacagcacgggcggctgggtgctcggcgttaggcgtccttggcacgggacagcaggggcgctgcgcggcgcggaacagcaggggtgcgggtccaccgggcgggtgctcctggacggcgcagggtcgacgggcgcgacaaccggtgggggcggcggcgcctgagatggaggagcagaacaaagccgcagtactgaacggagccgcagaggcgctgctggccgacacgaactgggccgctggcggggcgaggcggaggcgctgtggcgcgacagtcgcagctggccggcgtgggctgcgggcacgccgctgggtggagcaacgctacagcggcacgacaaactggaacaggcggagcgggcgacgccttcaggttgcggctgcgagtggcggcgaggcgcggcgctgcagactagaggcctgtttggtgggctggctcgggcgccacaccgcgccacactccggctgccgcgggtgtggcgcccaaatcggccgccgcaggtgtggcgggctgcggcgccgtaggctcctctccaaacaggccctagaaacgaaggggcgacgaccagcaggggctgcgcgcgggtggagcgtcgcgcgctggggccggggaagcagggccgtggggtcgcgtcggctgcaggcgcgagtggagaaggccaggcggcggcgctgcttgggggagaggagagaggaatggaagggagcagggcaggcggccagcagggggccgacggcgggagggaggccgacggctgggggccggcggctgggagggagaagggtgtggcggctgcaggggtggaaggagcccccggcggctgcagctactgtagaaaaaaaaactaaacctaggctgataccatgttggaatgtgaaccctaaccctaacaggggttgggtgatgtattaataggctgagtaaagtgggctaggcccattacagaggggtgagaaggtcattacacggggaagaccccaaaccctaaacgggtattctaacaagtatgaattgatgcaacattggggaacttgaatacaacatttgaatgaatctgtaactgattctttgacttttgctagtgccaatttaaaaacgtcatgtttgcctttttaacttccaggtatgtcatgtgttgatgatgtggacatttttgtgagttctttccctcatgaatgatttcaggtatacagcaatagggctgcctgttacaccaagctaggggcccttcctgaaggtctgaaagatgctgagaagtgcattgagctagatcccacattctccaagggatacacaaggaaaggtgctatccagtttttcatgaaagaatatgacaaagcgctggaaacttatcaagctggcctaaagcatgacccgaaaaaccaggagttacttgatggtgtaaggaggtaatgctgccggtggaagatcatgcacgtcttgtgatggtggtgtgtgtgctaagcagcaatctaatcgtcgctgatctttttcgtttcaggtgtgttgaacagatcaacaaggcgagcagaggcgaactcagtgaggaggaactgaaagagagacaggtaaaagtgttttctctaaaaatctttgttcggtaacgatggatagagtacttgccagtcgtttcgtccactactaatgcgagaagccaagaacacaatggctaacgggtcacctttgctatgcagaacaaagccatgcaagaccctgagatccagaacatcctgactgatccgatcatgcagcaggtaaggatagacgtctccctttgatttgaaatgcacataagtaggtgggcctgatggtgatctggtgatgccgagcttgccatgcaggttttgacggatctgcaggagaaccctcgggctgcgcaggcgcacctcaagaaccccggagtgatgcagaagatccagaagctcgtcagcgcagggatagttcagatgaagtagacggtggctggaatgtccgggttcagatctgggacctgtgctggaaatgctgtgaacttgatcattgtttcgcattttggaaactgaacccttttgtgcgaccatagtttggtcatggctgttaatgatccatatatgggttttcccatccgatatgttccaattcgtgcctttccccctggaatgcccgtgaaaaaggaagaatattcatcaaccttgcactcgagcagtttgtagagtcgtttaatgcgatggtatggtaccgtactaccgtggtaacatccagttgaacagcggcagaggtagtatttgcccccctcgtttgccaccggatagttactaggataatgattggtaccatccggataggcatcagggaacatatgaggtacactatgcaatcaagacgtgaaagtttttttttttaatattagtacataggaaaggtgagagtgagaggctgttttgaaacataatattatagatgaggtgatgtaaacataatagatggaaccatacatacatattagtagtgcaactatataaccaagacacaagtatataaatgagagtgaaatggttgtttaaaatat

SEQ ID NO: 64 - DNA, 1506 bp, Zea mays
  source 1..1506: mol_type = other DNA
atgacggaggaggcggaggcgaaggcgaagggcaacgcggcgttcgcggccggccgtttcgaggaggctgtccagcacttcagcgacgccatcgcgctcgcgcccgacaaccacgtcctctactccaaccgctcggcggcgtacgcgtcgctctggcgctacgcggaggcgctagatgacgccaagcggaccgtggcgctgaagccggactgggccaagggctactcccgcctcggcgccgcgcaccttggcctcggtgacgcgcccaaggccgtcgaggcctacgagaaagggcttgcgctcgagccgtccaatgaggcgctcaaatccggcctcgcacaggcgcgccagtccgcatcggcgccgagacgtccggtgggctccggggctgacgccgttggcaagatgttccagggccctgagctatggagcaaggtcgcggccgatcccaccacgcgtggctacctcgaccagccggacttcgtgcagatgctgcgggaggtgcagcgaaaccccggcagactgaacagctacctttctgaccaacgaatggttcaggtgctcaccctcatgctgaacatcaagttccagaaccagagcagtggggcgcctgacccggcagcagcacaatcgaacccgccgccaccaaagcagcagccagaggcgaaggcaagagaaccagagccggacccggagcccgagcctatggaggtgacagaggaggataaggagcagaaggagaggaaagcgtcggcgcagaaggagaaggaagcaggcaacgccgcttacaaaaggaaggattttgataccgcgatccagcactacacagaagccatggagctcgatgacgaggatatctcctacatcaccaatcgtgctgctgtttaccttgagatgggaaagtatgatgaatgcattaaggactgtgataaggctgtcgagagaggaagggaactgcatgctgatttcaagatgatctcaagggcacttaccagaaaaggaactgctcttgccaaacttgctaaatcatcgcaagactatgatgctgccattgagattttccagaaggcgttaactgagcaccggaacccagatacactgaaaaaactaaatgatgcagaacgagcaaagaaggagctggagcaacaagagtactatgacccaagaattgctgacgaggagcgagaaaaaggtaatgagttctttaagcagcagaaatatccagaagcagtgaaacattacaccgaagctctcaggagaaaccccaaggaccccagggtatacagcaatagggctgcctgttacaccaagctaggggcccttcctgaaggtctgaaagatgctgagaagtgcattgagctagatcccacattctccaagggatacacaaggaaaggtgctatccagtttttcatgaaagaatatgacaaagcgctggaaacttatcaagctggcctaaagcatgacccgaaaaaccaggagttacttgatggtgtaaggaggtaa

SEQ ID NO: 65 - protein, 501 aa, Zea mays
MTEEAEAKAKGNAAFAAGRFEEAVQHFSDAIALAPDNHVLYSNRSAAYASLWRYAEALDDAKRTVALKPDWAKGYSRLGAAHLGLGDAPKAVEAYEKGLALEPSNEALKSGLAQARQSASAPRRPVGSGADAVGKMFQGPELWSKVAADPTTRGYLDQPDFVQMLREVQRNPGRLNSYLSDQRMVQVLTLMLNIKFQNQSSGAPDPAAAQSNPPPPKQQPEAKAREPEPDPEPEPMEVTEEDKEQKERKASAQKEKEAGNAAYKRKDFDTAIQHYTEAMELDDEDISYITNRAAVYLEMGKYDECIKDCDKAVERGRELHADFKMISRALTRKGTALAKLAKSSQDYDAAIEIFQKALTEHRNPDTLKKLNDAERAKKELEQQEYYDPRIADEEREKGNEFFKQQKYPEAVKHYTEALRRNPKDPRVYSNRAACYTKLGALPEGLKDAEKCIELDPTFSKGYTRKGAIQFFMKEYDKALETYQAGLKHDPKNQELLDGVRR

SEQ ID NO: 66 - DNA, 735 bp, Zea mays
  source 1..735: mol_type = genomic DNA
atggaccctcacctcagctcccacgttcacgcggcagctgcggcggcctcggctttccccgatggtaagaagttggagagtgacgtgctgcctccaggggtcgtgaccctcgacgtcgaggcgggtgcgccgacgaacgcggccggggccgatccgtgcgacgccgacgacggggacgccggcttcgactacatggcccgcgcgcagtggctccgcgcggccgtcctcggcgcgaacgacggcctcgtctccgtggcctccctcatgatcggcatcggcgccgtgaacgcgacgcgcaaggccatgctggtctccggcatggcggggctcgtggccggcgcctgcagcatggccatcggcgagttcgtctccgtgtacgcgcagtacgacatcgaggtctcgcagatcaagcgcgacggcgaggaagccgccagggacagcctgccgagcccgacgcaggccgcgatcgcgtcggcgctggcgttcgcgtttggcgccctcctgccgctgctggcgggggtgttcgtaccgtcgtgggccgcaagggtggccgcggtgtgcgccgcgaccagcgtcggcctggccgggttcggcgtggcgggcgcgtacctgggcggcgccaacatgctgaggtccggcctgagggtgctcctgggcggctggttcgccatgctcgtcacgttcggtgtgctccggctgttcgggacggtcttccacatacaagtctcgtcggcg

SEQ ID NO: 67 - DNA, 735 bp, Zea mays; identical to SEQ ID NO: 66
  source 1..735: mol_type = other DNA

SEQ ID NO: 68 - protein, 735 aa, Zea mays
ATGGACCCTCACCTCAGCTCCCACGTTCACGCGGCAGCTGCGGCGGCCTCGGCTTTCCCCGATGGTAAGAAGTTGGAGAGTGACGTGCTGCCTCCAGGGGTCGTGACCCTCGACGTCGAGGCGGGTGCGCCGACGAACGCGGCCGGGGCCGATCCGTGCGACGCCGACGACGGGGACGCCGGCTTCGACTACATGGCCCGCGCGCAGTGGCTCCGCGCGGCCGTCCTCGGCGCGAACGACGGCCTCGTCTCCGTGGCCTCCCTCATGATCGGCATCGGCGCCGTGAACGCGACGCGCAAGGCCATGCTGGTCTCCGGCATGGCGGGGCTCGTGGCCGGCGCCTGCAGCATGGCCATCGGCGAGTTCGTCTCCGTGTACGCGCAGTACGACATCGAGGTCTCGCAGATCAAGCGCGACGGCGAGGAAGCCGCCAGGGACAGCCTGCCGAGCCCGACGCAGGCCGCGATCGCGTCGGCGCTGGCGTTCGCGTTTGGCGCCCTCCTGCCGCTGCTGGCGGGGGTGTTCGTACCGTCGTGGGCCGCAAGGGTGGCCGCGGTGTGCGCCGCGACCAGCGTCGGCCTGGCCGGGTTCGGCGTGGCGGGCGCGTACCTGGGCGGCGCCAACATGCTGAGGTCCGGCCTGAGGGTGCTCCTGGGCGGCTGGTTCGCCATGCTCGTCACGTTCGGTGTGCTCCGGCTGTTCGGGACGGTCTTCCACATACAAGTCTCGTCGGCG

SEQ ID NO: 69 - DNA, 735 bp, Zea mays; identical to SEQ ID NO: 66
  source 1..735: mol_type = genomic DNA

SEQ ID NO: 70 - DNA, 735 bp, Zea mays; identical to SEQ ID NO: 66
  source 1..735: mol_type = other DNA

SEQ ID NO: 71 - protein, 245 aa, Zea mays
MDPHLSSHVHAAAAAASAFPDGKKLESDVLPPGVVTLDVEAGAPTNAAGADPCDADDGDAGFDYMARAQWLRAAVLGANDGLVSVASLMIGIGAVNATRKAMLVSGMAGLVAGACSMAIGEFVSVYAQYDIEVSQIKRDGEEAARDSLPSPTQAAIASALAFAFGALLPLLAGVFVPSWAARVAAVCAATSVGLAGFGVAGAYLGGANMLRSGLRVLLGGWFAMLVTFGVLRLFGTVFHIQVSSA

SEQ ID NO: 72 - DNA, 1098 bp, Zea mays
  source 1..1098: mol_type = genomic DNA
gacagttctctcctgctcactcactcccttggccttctccagtcctctgtcctcgatcgatcggcttgtttacttccttagcttggagatccatttccatggaccctcacctcagctcccacgttcacgcggcagctgcggcggcctcggctttccccgatggtaagaagttggagagtgacgtgctgcctccaggggtcgtgaccctcgacgtcgaggcgggtgcgccgacgaacgcggccggggccgatccgcgcgacggcgacgacggggacgccggcttcgactacatggcccgcgcgcagtggctccgcgcggccgtcctcggcgcgaacgacggcctcgtctccgtggcctccctcatgatcggcatcggcgccgtgaacgcgacgcgcaaggccatgctggtctccggcatggcggggctcgtggccggcgcctgcagcatggccatcggcgagttcgtctccgtgtacgcgcagtacgacatcgaggtctcgcagatcaagcgcgacggcgaggaagccgccagggacagcctgccgagcccgacgcaggccgcgatcgcgtcggcgctggcgttcgcgtttggcgccctcctgccgctgctggcgggggtgttcgtaccgtcgtgggccgccagggtggccgcggtgtgcgccgcgaccagcgtcggcctggccgggttcggcgtggcgggcgcgtacctgggcggcgccaacatgctgaggtccggcctgagggtgctcctgggcggctggttcgccatgctcgtcacgttcggtgtgctccggctgttcgggacggtcttccacatacaagtctcgtcggcgtgatagctctcggagtctcgggctatggtggcaaatgaaataacgatccagggcccaggggcctagctatagctcttgacatcggaatgtgaaattaaaacctgcatgtcttgagtaaaaaactagcggtcgcctggatagcgaaatagaggcatgggtgtgggtgtgtccgtgtggcagtggatgtactgctttattgtaatgttatcttgtgctactggtatctgctgacgagaaaatcgaacacagattgtataattgtatt

SEQ ID NO: 73 - DNA, 738 bp, Zea mays
  source 1..738: mol_type = other DNA
atggaccctcacctcagctcccacgttcacgcggcagctgcggcggcctcggctttccccgatggtaagaagttggagagtgacgtgctgcctccaggggtcgtgaccctcgacgtcgaggcgggtgcgccgacgaacgcggccggggccgatccgcgcgacggcgacgacggggacgccggcttcgactacatggcccgcgcgcagtggctccgcgcggccgtcctcggcgcgaacgacggcctcgtctccgtggcctccctcatgatcggcatcggcgccgtgaacgcgacgcgcaaggccatgctggtctccggcatggcggggctcgtggccggcgcctgcagcatggccatcggcgagttcgtctccgtgtacgcgcagtacgacatcgaggtctcgcagatcaagcgcgacggcgaggaagccgccagggacagcctgccgagcccgacgcaggccgcgatcgcgtcggcgctggcgttcgcgtttggcgccctcctgccgctgctggcgggggtgttcgtaccgtcgtgggccgccagggtggccgcggtgtgcgccgcgaccagcgtcggcctggccgggttcggcgtggcgggcgcgtacctgggcggcgccaacatgctgaggtccggcctgagggtgctcctgggcggctggttcgccatgctcgtcacgttcggtgtgctccggctgttcgggacggtcttccacatacaagtctcgtcggcgtga

SEQ ID NO: 74 - protein, 245 aa, Zea mays
MDPHLSSHVHAAAAAASAFPDGKKLESDVLPPGVVTLDVEAGAPTNAAGADPRDGDDGDAGFDYMARAQWLRAAVLGANDGLVSVASLMIGIGAVNATRKAMLVSGMAGLVAGACSMAIGEFVSVYAQYDIEVSQIKRDGEEAARDSLPSPTQAAIASALAFAFGALLPLLAGVFVPSWAARVAAVCAATSVGLAGFGVAGAYLGGANMLRSGLRVLLGGWFAMLVTFGVLRLFGTVFHIQVSSA

SEQ ID NO: 75 - DNA, 1473 bp, Zea mays
  source 1..1473: mol_type = genomic DNA
gaccatggagcgatgaatttaaaaagtaccgagttgtcatggaatgtttgtagagggtttacccatactagccattactccacccttatccacacacacaaaaaggtacgcgacacaagccacgctctccgcttgtggctaagctgctgcacgcgcggaccgacaaaaatccagcactagccaaagcacaggcgaagcagcagcagcgagaggccaccacaaacatggcgaccacggcgtttctacccccggccaagctgccgctgcaggggcgtcggttcgctagccccaaaacggcgggacgtatcagtgtcagggtccctccggcgcgagcgcagcaagagcagcaggtgaaggaggaggaggccgaggtggcgagcgtgccgccgccgcaggccgcggagaaggaggcgaggaagggggacgcgcagtcgctgccgcggcagcctttggcggagagcaagaacatgagccgcgagtacggcgggcagtggctgagcagcaccacgcggcacgtccgcatctacgctgcgtacatcgacccggagaccaacgcgttcgaccagacgcagatggacaagctgacgctcatgctcgacccccaggacgagttcgcctggaccgacgaggcctgccagatggtcttcaacgagttccaggacctcgtcgatcactacgaggtaacctctgtagttgctcccacagtcccactccaacagctccgatagtagataaaaaaaatttgaattgaatctcttgttgtctgaaattcgtacggtcatttccccccttttcatctactttagagccactcctgccaaaaaaataaaacatacggtcttggcaaaagaagtgatcggtaagaggatagctgagctcgtttctgctttgaaacagggggctgagctgtcggagtacacacttcggttgatcgggtcagaccttgagcactacatccgcaaactgctgtacgacggggtgatcaagtacaacatgaggtccagagtcctcaatttcagcatgggcaagcctcgcatcaagttcaacagcaaccagataccagaaaagatatagaatgtacttatccaacaccctgaaaagaatggtttctctccattcgattaccgagcaagatgtatactctgcagtcccagatacgatccagagcattccagaattcatattacgcagatgcaaagccatacaccgattctgttggcaaaataagggtttgctcgagtttagataccccttctgtttctagggcactcgaattgggttacacgataccatagtttctgctaatagatacgtatcaccgaataccacatgaggtcagagagttgcacagagacgaccgcgctaacttaccaaacgactttctgaactggatggagtaatggctagtatgggtaaaccctctacaaacattccatgacaactcggtactttttaaattcatcgctccatggt

SEQ ID NO: 76 - DNA, 633 bp, Zea mays; identical to SEQ ID NO: 37
  source 1..633: mol_type = other DNA

SEQ ID NO: 77 - protein, 210 aa, Zea mays; identical to SEQ ID NO: 38

SEQ ID NO: 78 - DNA, 1286 bp, Zea mays
  source 1..1286: mol_type = genomic DNA
ccaaagcacaggcgaagcagcagcagcgagaggccaccacaaacatggcgaccacggcgtttctacccccggccaagctgccgctgcaggggcgtcggttcgctagccccaaaacggcgggacgtatcagtgtcagggtccctccggcgcgagcgcagcaagagcagcaggtgaaggaggaggaggccgaggtggcgagcgtgccgccgccgcaggccgcggagaaggaggcgaggaagggggacgcgcagtcgctgccgcggcagcctttggcggagagcaagaacatgagccgcgagtacggcgggcagtggctgagcagcaccacgcggcacgtccgcatctacgctgcgtacatcgacccggagaccaacgcgttcgaccagacgcagatggacaagctgacgctcatgctcgacccccaggacgagttcgcctggaccgacgaggcctgccagatggtcttcaacgagttccaggacctcgtcgatcactacgaggtaacctctgtagttgctcccacagtcccactccaacagctccgatagtagataaaaaaaatttgaattgaatctcttgttgtctgaaattcgtacggtcatttccccccttttcatctactttagagccactcctgccaaaaaaataaaacatacggtcttggcaaaagaagtgatcggtaagaggatagctgagctcgtttctgctttgaaacagggggctgagctgtcggagtacacacttcggttgatcgggtcagaccttgagcactacatccgcaaactgctgtacgacggggtgatcaagtacaacatgaggtccagagtcctcaatttcagcatgggcaagcctcgcatcaagttcaacagcaaccagataccagaaaagatatagaatgtacttatccaacaccctgaaaagaatggtttctctccattcgattaccgagcaagatgtatactctgcagtcccagatacgatccagagcattccagaattcatattacgcagatgcaaagccatacaccgattctgttggcaaaataagggtttgctcgagtttagataccccttctgtttctagggcactcgaattgggttacacgataccatagtttctgctaatagatacgtatcaccgaataccacatgaggtcagagagttgcacagagacgaccgcgctaacttaccaaacgactttctgaactggatggagtaatggctagtatgggtaaaccctctacaaacattccatgacaactcggtactttttaaattcatcgct

SEQ ID NO: 79 - DNA, 633 bp, Zea mays; identical to SEQ ID NO: 37
  source 1..633: mol_type = other DNA

SEQ ID NO: 80 - protein, 210 aa, Zea mays; identical to SEQ ID NO: 38

SEQ ID NO: 81 - DNA, 25 bp, synthetic construct
  source 1..25: mol_type = other DNA
accctgaaaagaatggtttctctcc

SEQ ID NO: 82 - DNA, 25 bp, synthetic construct
  source 1..25: mol_type = other DNA
tatggctttgcatctgcgtaatatg

SEQ ID NO: 83 - DNA, 20 bp, synthetic construct
  source 1..20: mol_type = other DNA
tgtactcggcaatgctcttg

SEQ ID NO: 84 - DNA, 20 bp, synthetic construct
  source 1..20: mol_type = other DNA
tttgatgctccaggcttacc

SEQ ID NO: 85 - DNA, 100 bp, Zea mays
  source 1..100: mol_type = genomic DNA
cttggacttgcctcaatggaaattaaattattggtaaaccactaaatctttggataaaaatcatcatgtcaaagtctcttcatatttttgttagtttctt

SEQ ID NO: 86 - DNA, 99 bp, Zea mays
  source 1..99: mol_type = genomic DNA
cttggacttgcctcaatggaaattaaattattggtaaaccactaaatctttggataaaatcatcatgtcaaagtctcttcatatttttgttagtttctt

SEQ ID NO: 87 - DNA, 100 bp, Zea mays
  source 1..100: mol_type = genomic DNA
cttggacttgcctcaatgggaattaaattattggtaaaccactaaatcttaggataaaaatcatcatgtcaaagtctcttcatatttatgttagtttctt

SEQ ID NO: 88 - DNA, 20 bp, synthetic construct
  source 1..20: mol_type = other DNA
accgaagtgtgtactccgac

SEQ ID NO: 89 - DNA, 20 bp, synthetic construct
  source 1..20: mol_type = other DNA
aacggcccttatccacacac

SEQ ID NO: 90 - DNA, 413 bp, synthetic construct
  source 1..413: mol_type = other DNA
cccttccctcttcgtccataatggcaattatctcagcagcgagaggccaccacaaacatggcgaccacggcgtttctacccccggccaagctgccgctgcaggggcgtcggttcgctagccccaaaacggcgggacgtatcagtgtcagggtccctccggcgcgagcgcagcaagagcagcaggtgaaggaggaggaggccgaggcggcgagcgtgccgccgccgcaggccgcggagaaggaggcgaggaagggggacgcgcagtccctgccgcggcagcctttggcggagagcaagaacatgagccgcgagtacggcgggcagtggctgagcagcaccacgcggcacgtccgcatctacgctgcgtacatcgacccggtgaccaacgcgttcgaccagacgcagatggacaa

SEQ ID NO: 91 - DNA, 417 bp, synthetic construct
  source 1..417: mol_type = other DNA
ttagagaagccaacgccatcgcctcctatttcgtcgaatccccttccctcttcgtccataatggcaattatctcagcagcgagaggccaccacaaacatggcgaccacggcgtttctacccccggccaagctgccgctgcaggggcgtcggttcgctagccccaaaacggcgggacgtatcagtgtcagggtccctccggcgcgagcgcagcaagagcagcaggtgaaggaggaggaggccgaggcggcgagcgtgccgccgccgcaggccgcggagaaggaggcgaggaagggggacgcgcagtccctgccgcggcagcctttggcggagagcaagaacatgagccgcgagtacggcgggcagtggctgagcagcaccacgcggcacgtccgcatctacgctgcgtacatcgaccc

SEQ ID NO: 92 - DNA, 422 bp, synthetic construct
  source 1..422: mol_type = other DNA
ccggaacccttcccttcttcgtccataatgggcaattatctcagcagcgagaggccaccacaaacatggcgaccacggcgtttctacccccggccaagctgccgctgcaggggcgtcggttcgctagccccaaaacggcgggacgtatcagtgtcagggtccctccggcgcgagcgcagcaagagcagcaggtgaaggaggaggaggccgaggcggcgagcgtgccgccgccgcaggccgcggagaaggaggcgaggaagggggacgcgcagtccctgccgcggcagcctttggcggagagcaagaacatgagccgcgagtacggcgggcagtggctgagcagcaccacgcggcacgtccgcatctacgctgcgtacatcgacccggtgaccaacgcgttcgaccagacgcagatggacaaa

(19) United States
(12) Patent Application Publication
(10) Pub. No.: US 2026/0215383 A1
(43) Pub. Date: Jul. 30, 2026

(54) PLANTS WITH IMPROVED PERFORMANCE

(71) Applicants: KWS Saat SE & Co. KGaA, Einbeck (DE); Technische Universität München, München (DE); Heinrich-Heine-Universität Düsseldorf, Düsseldorf (DE)

(72) Inventors: Daniela SCHEUERMANN, Einbeck (DE); Claude URBANY, Einbeck (DE); Milena OUZUNOVA, Göttingen (DE); Thomas PRESTERL, Einbeck (DE); Manfred MAYER, Bad Füssing (DE); Chris-Carolin SCHÖN, München (DE); Sebastian URZINGER, Freising (DE); Peter WESTHOFF, Neuss (DE); Karin ERNST, Wuppertal (DE)

(21) Appl. No.: 19/159,066

(22) PCT Filed: Jan. 29, 2024
(86) PCT No.: PCT/EP24/52094

§ 371 Date: Aug. 22, 2025

(30) Foreign Application Priority Data
Feb. 22, 2023 (EP) 23157908.7

(51) Int. Cl. A01H 1/04; A01H 1/00; C12N 9/22; C12N 15/11; C12N 15/82; C12Q 1/6895

(52) U.S. Cl. CPC A01H 1/045; A01H 1/1225; C12N 9/222; C12N 15/111; C12N 15/8201; C12N 15/8273; C12Q 1/6895; C12N 2310/20; C12Q 2600/13

(57) ABSTRACT

Described herein are methods of identifying maize plants with an improved early plant vigor and early plant height. Further, described herein is a specific set of markers diagnostic for a chromosomal region of about or less than 0.68 Mb on chromosome 2 of Zea mays, the region defining a quantitative trait locus (QTL) locus that affects sensitivity to cold stress, early vigor and early plant height at stages V4 and V6 and maximum potential quantum efficiency of photosystem II. Also described herein are methods and means for detecting the desired phenotype. Finally, described herein are methods of using the QTL-bearing chromosomal region for the manufacture and/or identification of a plant of the genus Zea having early plant vigor.

### TECHNICAL FIELD

The present invention relates to the field of identifying maize plants with an improved early plant vigor and early plant height. Further, there is provided a specific set of markers diagnostic for a chromosomal region of about or less than 0.68 Mb on chromosome 2 of Zea mays, said region defining a quantitative trait locus (QTL) locus that affects sensitivity to cold stress, early vigor and early plant height at stages V4 and V6 and maximum potential quantum efficiency of photosystem II. Methods and means for detecting the desired phenotype are also provided. Finally, uses of the QTL-bearing chromosomal region for the manufacture and/or identification of a plant of the genus Zea having early plant vigor are provided.

### BACKGROUND

Maize (Zea mays L.), being a C4 plant of tropical origin, is sensitive to chilling. The expression “chill” means temperatures at which the maize plant can survive, but the growth is compromised or even substantially compromised. The optimal growth temperature for the germination of maize seeds and the development of maize plants is between 21-27° C. (Greaves, 1996).

Thus, stress already occurs below temperatures of 20° C., which is a typical temperature in Northern Europe during planting times. Mild chilling stress with reduced photosynthesis in light and reduced growth is seen at 12-17° C., and severe chilling stress occurs together with cold-induced water stress, a type of drought stress, in light at 2-10° C. (Marocco et al., 2005).

Chilling stress in maize (Zea mays L.) burdens plant development and in consequence decreases yield. Chill stress is accompanied by either photoinhibition and oxidative stress in light or gene expression alterations in the dark (summarized in Marocco et al., 2005). The heterotrophic phase (sowing up to the third leaf) is the most sensitive, but the early autotrophic phase is also affected by chill stress (Bhosale et al., 2007). The long-term action of low temperatures results in irreversible damage to the cells and tissue (Greaves, 1996) and is associated with reduced growth and yield.

Especially in the early seedling stage, various developmental and physiological processes are affected by chilling stress (Greaves 1996; Marocco et al. 2005). Notably, photosynthesis is impaired and a chlorotic phenotype manifests (Baker and Nie 1994; Foyer et al. 2002; Fryer et al. 1998; Leipner et al. 1999; Marocco et al. 2005). This is mainly caused by an overexcitation of the phytochromes and a concomitant production of oxygen radicals.

Correspondingly, early plant vigor is regarded as an important trait for maize grown in the cool environments of Central and Northern Europe. Improving cold tolerance in maize would allow early sowing for avoiding summer drought, diseases, and pests (Kucharik 2006). Additionally, early sowing could potentially increase yield by prolonging growth cycle (Revilla et al. 2005; Strigens et al. 2012). Classical plant breeding has resulted in a slow but steady improvement of this trait in Central Europe (Frei 2000).

Mayer et al., 2020 describes a genome wide association study (GWAS) for early development traits. The study identified many regions associated with early vigor and plant height across all chromosomes with some intervals having a size of several Mb. Due to the sheer size of such large regions, these are not well suited for molecular breeding. As multiple recombination events occur regularly within such a region, it is not stably inherited as a whole and markers flanking the interval or at a random position within cannot reliably predict the desired traits. Moreover, since the actual locus causal for the improved traits within such an interval may well be over 1000 times smaller than the entire region, using the complete interval may have various undesirable effects, resulting from numerous different loci therein unrelated or even adversely related to the desired traits. In addition, such a large region is impossible to use for the production of plant lines by genomic engineering, as this would require e.g. a discrete gene to be inserted or a discrete position to be modified. Furthermore, while the analysis in Mayer et al., 2020 assesses early plant vigor and plant height, chilling tolerance and photosynthesis were never addressed. Hence, there is no indication, which of the many regions, if any, could potentially be associated with these traits.

It was thus an object of the present invention to provide a narrow, well-defined and verified, quantitative trait locus (QTL) associated with improved traits of abiotic stress tolerance, including chilling tolerance, early vigor, early plant height, and/or quantum efficiency (as a measure of photosynthesis). Moreover, it was a further objective to provide one or more genes and/or discrete genomic sequences associated with the desired traits, which may not only be used for identification and selection plants but also for the production of plant lines, e.g. by genome editing.

In view of the complexity of crop plant genomes, a successful identification of novel traits, e.g., by identifying a causative gene or QTL, and the characterization of the phenotypic effect is tremendously cumbersome. Moreover, even the availability of a locus or predictions and extrapolations starting from known candidate genes and loci frequently fail in the context of a complex plant genome in a typical breeding set-up.

The inventors have identified and localized a narrow QTL of 0.68 Mb on maize chromosome 2 contributing to genetic variation for improved early vigor and early plant height, in particular at stages V4 and V6, maximum potential quantum efficiency of Photosystem II and tolerance to cold treatment. This QTL region was characterized on the sequence level and its phenotypic effect at the molecular, biochemical and physiological level is described herein. Candidate genes for the phenotypic effects of the QTL region were identified, and functional validation studies based on mutant plants and gene expression studies conducted. The analysis showed that functional expression of the gene ZmNdhM (Zm00001eb075370) within the QTL positively correlates with all four desired traits.

### SUMMARY OF THE INVENTION

In a first aspect, there is provided a method for identifying and/or selecting a plant of the genus Zea having an improved early vigor and/or early plant height phenotype, preferably due to an improved abiotic stress tolerance, including chilling tolerance, and/or an improved quantum efficiency, the method comprising a) providing a plant or a plant population of the genus Zea comprising genomic DNA; and b1) detecting the presence or absence of one, two, three or more QTL allele(s) comprising at least one allele being selected from a “G” at position 23261992, and/or an “A” at position 23478077, and/or a “G” at position 24154201, wherein the positions refer to chromosome 2 and are defined according to the Zea mays physical map B73_AGPv05 reference annotation, preferably wherein the one, two, three or more QTL allele(s) is/are associated with an improved early vigor and/or early plant height phenotype; and/or b2) detecting the presence or absence of at least one functional copy of Zm00001eb075370, and/or a functional fragment, ortholog, homolog or paralog thereof, preferably wherein expression of said gene encoding at least one functional copy of Zm00001eb075370 is about 50% to about 150%, more preferably about 80% to about 120% or about the same level as Zm00001eb075370 expression as in Kemater DH line KE0482 deposited as NCIMB 44328; and c) optionally: selecting at least one plant as having an improved early vigor and/or early plant height phenotype if the one or more of the QTL alleles of b1) and/or the at least one functional copy of Zm00001eb075370 of b2) is/are present.

In one embodiment of the first aspect, the method comprises an additional step of obtaining at least one plant, or plant cell, seed, tissue, and/or organ thereof identified as having an improved early vigor and/or early plant height phenotype based on the detection step b1) and/or b2), preferably by directly obtaining said at least one plant, plant cell, seed, tissue, and/or organ in step c) of selecting at least one plant as having an improved early vigor and/or plant height phenotype if the one or more of the QTL alleles of b1) and/or the at least one functional copy of Zm00001eb075370 of b2) is/are present. “Directly obtaining” in this context is to be understood as meaning that said at least one plant, plant cell, seed, tissue, and/or organ is directly obtained during the selection step as the immediate result of screening and selecting by selecting exactly said at least one plant, plant cell, seed, tissue, and/or organ of the same generation as the material screened, optionally including a step of asexual callus regeneration or propagation of the material, but without a further step of crossing.

In some embodiments of the first aspect, the presence or absence of the one, two, three or more QTL alleles is detected using one, two, three or more markers selected from SEQ ID NO: 1 to SEQ ID NO: 8, or a sequence having at least 95%, at least 96%, at least 97%, at least 98%, or at least 99% sequence identity thereto, preferably using at least one marker selected from SEQ ID NO: 4 and/or 5 or a sequence having at least 95%, at least 96%, at least 97%, at least 98%, or at least 99% sequence identity thereto.

In some embodiments of the first aspect, the method comprises the detection of an “A” at position 23478077 as defined above.

In some embodiments of the first aspect, the method comprises the detection of a “G” at position 24154201 as defined above.

In some embodiments of the first aspect, the method comprises the detection of an “A” at position 23478077 and a “G” at position 24154201 as defined above.

In some embodiments of the first aspect, the plant of the genus Zea is a Zea mays plant.

In a second aspect, there is provided a method of producing a plant of the genus Zea having an improved early vigor and/or early plant height phenotype, the method comprising the (a) introduction of, and/or (b) mutagenesis to confer, preferably chemical- or radiation-induced mutagenesis, and/or targeted mutagenesis, at least one functional copy of Zm00001eb075370, and/or a functional ortholog, homolog or paralog thereof, optionally wherein the plant of the genus Zea is a Zea mays plant.

In some embodiments of the second aspect, the method comprises increasing the expression of Zm00001eb075370, or a functional fragment, ortholog, homolog or paralog thereof, in at least one plant cell.

In one embodiment of the second aspect, the method comprises the introduction of at least one nucleic acid molecule comprising or consisting of a nucleic acid sequence according to SEQ ID NO: 39, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto, or of a functional fragment, ortholog, homolog or paralog thereof, or a nucleic acid molecule encoding any one of SEQ ID NOs: 35, 38, 41, 44, 47, 77 or 80, or a sequence having at least 90%, 91%, 92%, 93%, 94%, 95%, 96%, 97%, 98% or at least 99% sequence identity thereto into at least one plant cell.

In another embodiment of the second aspect, the method comprises targeted mutagenesis of at least one plant cell to confer at least one functional copy of Zm00001eb075370 and/or a functional ortholog, homolog or paralog, optionally wherein the targeted mutagenesis is SDN1 genome editing.

In a third aspect, there is provided a transgenic plant, or plant cell, seed, tissue, and/or organ thereof of the genus Zea, optionally a Zea mays plant, or plant cell, seed, tissue, and/or organ thereof, comprising at least one functional copy of Zm00001eb075370 and/or a functional fragment, ortholog, homolog or paralog thereof.

In a fourth aspect, there is provided a use of one, two or more marker(s) capable of detecting the detecting the presence or absence of one, two, three or more of the QTL alleles as defined in claim 1 for identifying and/or selecting a plant of the genus Zea having an improved early vigor and/or early plant height phenotype, preferably wherein the use comprises a use of a marker capable of detecting the detecting the presence or absence of A″ at position 23478077, and/or a marker capable of detecting “G” at position 24154201, optionally a marker capable of detecting the detecting the presence or absence of A″ at position 23478077 and a of a marker capable of detecting “G” at position 24154201, wherein the positions are as defined in above, preferably wherein the one, two or more marker(s) is/are independently selected from SEQ ID NO: 1 to SEQ ID NO: 8, or a sequence having at least 95%, at least 96%, at least 97%, at least 98%, or at least 99% sequence identity thereto, preferably using at least one marker selected from SEQ ID NO: 4 and/or 5 or a sequence having at least 95%, at least 96%, at least 97%, at least 98%, or at least 99% sequence identity thereto.

In some embodiments of the fourth aspect, the use comprises the use of one, two or three or more markers selected from SEQ ID NO: 1 to SEQ ID NO: 8, or a sequence having at least 95%, at least 96%, at least 97%, at least 98%, or at least 99% sequence identity thereto, preferably using at least one marker selected from SEQ ID NO: 4 and/or 5 or a sequence having at least 95%, at least 96%, at least 97%, at least 98%, or at least 99% sequence identity thereto.

In a fifth aspect, there is provided a use of a QTL on chromosome 2, or at least one part thereof, associated with early vigor and/or plant height, flanked by positions 23261992 and 24154201, preferably flanked by positions 23478077 and 24154201, defined according to the Zea mays physical map B73_AGPv05 reference annotation, and/or a use of a functional copy of Zm00001eb075370 of Zm00001eb075370, and/or a functional ortholog, homolog or paralog thereof, for the identification, selection and/or manufacture of a plant of the genus Zea, including a Zea mays plant, having an improved early vigor and/or early plant height phenotype.

### Definitions

Unless defined otherwise, technical and scientific terms used herein have the same meaning as commonly understood by one of ordinary skill in the art to which this invention belongs.

The term “about” means+/−10% of the recited value, preferably +/−5% of the recited value.

The term “AGPv05 reference annotation” refers to the physical map (B73 v5 reference genome) from maize as described in (Hufford et al., 2021). Unless specifically stated otherwise, numbers specifying one or more positions on a chromosome are always given in base pairs (bp) and always refer the chromosome 2 of the AGPv05 reference annotation/genome. Distances spanning from a first position to a second position, i.e. the flanking positions, are, unless stated otherwise, given in the unit megabase(s) (Mb), wherein windows and/or regions specified herein always include the flanking positions. Consequently, a region defined as “flanked by”, refers to the interval comprising the stretch between the flanking positions and the flanking positions themselves. Notably, the B73v5 genome can be obtained from MaizeGDB's Download section under https://download.maizegdb.org/Zm-B73-REFERENCE-NAM-5.0/. The corresponding genome assembly can be assessed from https://download.maizegdb.org/Zm-B73-REFERENCE-NAM-5.0/Zm-B73-REFERENCE-NAM-5.0.fa.gz.

The term “allele” as used herein refers to a nucleic acid sequence variant at a specific location, such as an allele of a single nucleotide polymorphism. Generally, an allele can be understood as any one of two or more genes and/or loci that may occur alternatively at a given site on a chromosome. Alleles may occur in pairs, or there may be multiple alleles affecting the expression (phenotype) of a particular trait.

The term “allelic variation” as used herein describes the presence or number of different allele forms at a particular locus on a chromosome.

An allele, “associated with” an improved trait as disclosed herein, directly and/or indirectly confers or contributes to an increase and/or improvement in for respective trait, compared to at least one different allele, such as alleles “B” in Table 2. This includes sequence variants that—themselves—directly and/or indirectly cause the conferral of and/or contribution to improvement in for respective trait, but also includes alleles, e.g. marker alleles, that are indicative of one or more sequence variants that directly and/or indirectly cause the conferral of and/or contribution to improvement in for respective trait. A QTL “associated with” an improved trait as disclosed herein, refers to a QTL comprising one or more polymorphic sites for which at least one allele is associated with the respective improved trait. A gene “associated with” an improved trait, as disclosed herein, is a gene, which has at least one allele, i.e. a sequence variant of the gene sequence itself, that is “associated with” the respective improved trait or which is otherwise involved in the association of one or more alleles associated with the respective improved trait, e.g. the expression of which may affected by one or more alleles “associated with” an improved trait as disclosed herein.

“Chilling tolerance” or “cold tolerance”, e.g., in the context of maize being a C(4) plant species with higher temperature optima than C(3) plant species, refers to a higher tolerance of a plant in comparison to a reference plant towards chilling or cold conditions, e.g., during outgrowth, but also during regular growth. Chilling stress is a complex phenomenon with physiological and biochemical responses at both cellular and whole-organ level, e.g., influencing CO₂ assimilation rates of plants. In maize, chilling stress is particularly known to reduce leaf size, stem extension and root proliferation, disturb plant water relations, and impede nutrient uptake so that chilling tolerance is a highly desirable phenotype.

Plant height is the height of a free-standing plant, measured from the soil surface to its highest tip when all leaves are stretched upwards.

Plant vigor describes the strength of a plant by its visual appearance. Plant vigor considers the biomass (height, width, leaf area), color (greenness, discolorations) and health (necrosis, general appearance) of a plant. Plant vigor is scored on a scale of 1-9 that is given by visual assessment of a plant. A score of 1 means a plant has a very low vigor, (small, discolored, weak) and a score of 9 meaning it is very vigorous (big, green, strong).

The terms “quantum efficiency” or “Fv/Fm” are used interchangeably herein and refer to the trait of maximum quantum yield efficiency of photosystem 2 (Fv/Fm), i.e. the ratio of the number of molecules reacted or formed and the number of photons absorbed by photosystem 2.

A “functional copy of Zm00001eb075370” as used herein, refers to a variant of Zm00001eb075370, being the only variant thereof within a plant genome or being one of two or more variants thereof within an plant genome, that allows expression of Zm00001eb075370 that is about 50% to about 150%, preferably about 80% to about 120% or about the same level as Zm00001eb075370 expression as in Kemater DH line KE0482 deposited as NCIMB 44328, as measurable on transcript level or protein level, preferably as measurable on transcript level, wherein the expression may be quantified as disclosed herein or by any other suitable method known in the art; preferably wherein the intron sequence of a functional the copy of Zm00001eb075370 may not comprise an insertion, relative to the intron sequence of ZmNdhM according to positions 672 to 897 of SEQ ID NO: 39, that is larger than 100 bp, 50 bp, 30 bp or 10 bp; and/or wherein the intron sequence of a functional the copy of Zm00001eb075370 may not comprise an insertion, relative to the sequence SEQ ID NO 39, between the nucleotides according to positions 710 and 718 of SEQ ID NO 39 that is larger than 100 bp, 50 bp, 30 bp or 10 bp, optionally wherein the intron sequence does not comprise an insertion between the nucleotides according to positions 710 and 718 of SEQ ID NO 39; and/or wherein the intron sequence of a functional copy of Zm00001eb075370 may not comprise an insertion between the nucleotides according to positions 23631857 to 23631867 according to the AGPv05 reference annotation that is larger than 100 bp, 50 bp, 30 bp or 10 bp, optionally wherein the intron sequence does not comprise an insertion between the nucleotides according to positions 23631857 to 23631867 according to the AGPv05 reference annotation; and/or wherein the intron sequence of a functional the copy of Zm00001eb075370 may comprise or consist of an intron sequence according to positions 672 to 897 of SEQ ID NO: 39 or a sequence having least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto; and/or wherein the sequence of a functional the copy of Zm00001eb075370 may comprise or consist of a sequence according to SEQ ID NOs: 39, or a sequence having least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

A “functional ortholog, homolog or paralog of Zm00001eb075370” refers to any gene other than Zm00001eb075370 that encodes a polypeptide according to any one of SEQ ID NOs: 35, 38, 41, 44, 47, 77 or 80, or a sequence having at least 90%, 91%, 92%, 93%, 94%, 95%, 96%, 97%, 98% or at least 99% sequence identity thereto, wherein expression of said gene that is about 50% to about 150%, preferably about 80% to about 120% or about the same level as Zm00001eb075370 expression as in Kemater DH line KE0482 deposited as NCIMB 44328, as measured on transcript level or protein level, preferably as measured on transcript level, wherein the expression may be quantified as disclosed herein or by any other suitable method known in the art. A “functional ortholog, homolog or paralog of Zm00001eb075370” may for example be a sequence according to SEQ ID NOs: 75 without being limited thereto. Transcription and/or expression levels to define a certain level of transcription or expression are known to the skilled person. Suitable examples are exemplified below and shown in FIG. 5 showing quantitative proteomics data for NDHM, wherein they-axis refers to observed reporter ion intensities associated with the NDHM protein expression level. On RNA level, as a high-throughput alternative to RT-qPCR, Gene Expression Assays can be used, for example with an assay available at ThermoFisher (https://www.thermofisher.com/de/de/home/life-science/gene-expression-analysis-aenotypin/quantigene-rna-assays/quantigene-singleplex-assay/quantigene-singleplex-ht-assay.html). On protein level, quantification of expression levels can be performed, for example, by antibody-based methods, e. g. ELISA (HNASKO, Robert (Hg.). Elisa. New York, NY, USA: Humana Press, 2015.). For Arabidopsis, a NDHM antibody is readily available (https://www.biorbyt.com/ndhm-antibody-orb792644.html). On Protein level quantification could be performed by antibody based methods, e. g. ELISA (HNASKO, Robert (Hg.). Elisa. New York, NY, USA: Humana Press, 2015.). For Arabidopsis NDHM antibody is readily available (https://www.biorbyt.com/ndhm-antibody-orb792644.html).

The term “genetic variation” as used herein describes the presence and/or number of differences in sequences of genes between individual organisms of a species.

The terms “genome modification” and “genome editing” are used interchangeably herein.

The term “haplotype” refers to the genotype of an individual at a plurality of loci, i.e. a combination of more than one allele, e.g. more than one of the alleles defined in Table 1.

“Improved” quantitative traits, such as chilling tolerance, early vigor, early plant height and/or quantum efficiency, refers to an increase, preferably an increase of at least 5%, 10%, 15%, 20%, 25%, 30%, 30%, 40%, 45%, or at least 50%, optionally up to 200%, in the respective trait, wherein the improved trait refers to an increase in any of said traits as conferred or conferrable by one or more alleles associated with the improved trait(s), as disclosed herein, in comparison to a plant not comprising said one or more allele(s), in an otherwise, at least outside of the specified QTL, identical or nearly identical genetic background, i.e. the genotype outside of the specified QTL being identical or nearly identical, if grown under the same conditions within reasonable limits. The traits of plant height, plant vigor and quantum efficiency may be improved under and/or after cold conditions (about 2° C. to 20° C.) and/or at control conditions (about 21° C. to 27° C.), preferably the traits are improved under cold conditions, after cold conditions and under control conditions. Generally, plant height, plant vigor and quantum efficiency may be subject to a stronger improvement by the QTL of the present invention under and/or after cold conditions.

An improved trait, as disclosed herein, encompasses situations in which one or more alleles associated with the improved trait are present and/or introduced, e.g. through crossing, by mutagenesis, by targeted genome editing or as a transgene, wherein the improved trait conferred by said one or more allele(s) may not be directly phenotypically visible due to other alleles, e.g. outside of the defined QTL, negatively affecting one the trait that may also have been introduced, and the improved trait may only become visible, for instance, though backcrossing to remove such adverse alleles. The traits disclosed herein can be quantitatively assessed as disclosed herein or may be measured by any suitable method used in the art, as long as samples to be compared are assessed using the same measurement.

An improved “early” trait as used herein, refers to an improvement of said trait at least at the 6-leaf-stage (V6; see e.g. Zhao et al 2011), optionally at least at and/or between the 4-leaf-stage (V4) and/or the 6-leaf-stage (V6). “Improved early vigor and/or plant height” as used herein, refers to improved early vigor and/or improved early plant height.

The term “insertion” as used herein refers to the presence of one or more additional nucleotides or nucleotide base pairs in one nucleotide sequence relative to another nucleotide sequence, i.e. the number of nucleotides or nucleotide base pairs in said sequence is increased relative to said other sequence at the specified region comprising an insertion. In insertion may be present in combination with one or more nucleotide exchanges, i.e. the presence of a different nucleotide at a certain position. Irrespective of possible nucleotide exchanges, an insertion is present (relative to a different nucleotide sequence) within a certain region, if the number of nucleotides or nucleotide base pairs in said region is increased (relative to the corresponding region of said different nucleotide sequence).

The term “marker” as used herein refers to a position and/or sequence comprising a variation, such as a single nucleotide polymorphism (SNP) that may be used for genetic mapping and identification of desirable traits. Markers disclosed in SEQ ID NOs: 1 to 8 comprise a SNP. The location within chromosome 2 of the respective polymorphic site shown in any one of SEQ ID NOs: 1 to 8 is indicated in Table 1. Naturally, to detect the markers, it is not required to detect the entire sequence as disclosed in any one of SEQ ID NOs: 1 to 8. It is sufficient to detect the allele of the polymorphic site, in this case the respective SNP. Alleles of polymorphic sites of a population can be detected by various methods well known in the art. These include, e.g., PCR-based and/or amplification-based methods, such as competitive allele specific PCR (KASP; Semagn et al 2014), e.g. with allele-specific primers as disclosed in SEQ ID NOs: 9 to 32, restriction fragment length polymorphisms (RFLP/PCR-RFLP) detection (Hashim and Al-Shuhaib 2019, single-strand conformation polymorphism (SSCP/PCR-SSCP) detection (Hashim and Al-Shuhaib 2019), allele specific hybridization (ASH; Poulsen et al., 2011), or allele-specific oligonucleotide (ASO) hybridization, detection of simple sequence repeats (SSRs; Qu and Liu 2013), or conventional sequencing methods, such as sanger sequencing or next generation sequencing, including high-throughput sequencing, and the likes. The skilled person is well aware of the design and use of various different types of nucleic acid molecules for the detection of alleles and/or genotyping of nucleic acid sequences.

The terms “plant” as used herein refer to a plant organism, a plant organ, differentiated and undifferentiated plant tissues, plant cells, seeds, and derivatives and progenies thereof. Plant cells include without limitation, for example, cells from seeds, from mature and immature embryos, meristematic tissues, seedlings, callus tissues in different differentiation states, leaves, flowers, roots, shoots, male or female gametophytes, sporophytes, pollen, pollen tubes and microspores, protoplasts, macroalgae and microalgae. Different plant cells can have any degree of ploidy, i.e. they may be either haploid, diploid, tetraploid, hexaploid or polyploid.

The term “regulatory region” as used herein refers to a nucleic acid sequence that is not part of the protein-encoding nucleotide sequence, but can direct and/or influence the expression of the protein-encoding nucleotide sequence. The term thus refers to core promoter sequence, a proximal promoter sequence, a terminator sequences or polyadenylation signals and the like. Other examples of regulatory sequences are enhancers, silencers, insulators, tethering elements, introns, or cis-regulatory regions, trans regulatory regions or a locus control sequence. A regulatory my also influence splicing of a particular nucleic acid sequence, for instance regulating alternative splicing variants. A regulatory sequence can further be a combination of the above. Depending on the type of regulatory region it is located on the nucleic acid molecule before (i.e., 5′ of), after (i.e., 3′ of) or between (in case of intronic elements) the protein-encoding nucleotide sequence.

The terms “SDN-1”, “SDN-2”, and “SDN-3” as used herein are abbreviations for the platform technique “site-directed nuclease” 1, 2, or 3, respectively, as caused by any site-directed nuclease of interest, including, for example, Meganucleases, Zinc-Finger Nucleases (ZFNs), Transcription Activator Like Effector Nucleases (TALENs), and CRISPR nucleases. SDN-1 produces a double-stranded or single-stranded break in the genome of a plant without the addition of foreign DNA. A “site-directed nuclease” is thus able to recognize and cut, optionally assisted by further molecules, a specific sequence in a genome or an isolate genomic sequence of interest. For SDN-2 and SDN-3, an exogenous nucleotide template is provided to the cell during the gene editing. For SDN-2, however, no recombinant foreign DNA is inserted into the genome of a target cell, but the endogenous repair process copies, for example, a mutation as present in the template to induce a (point) mutation. In contrast, SDN-3 mechanism uses the introduced template during repair of the DNA break so that genetic material is introduced into the genomic material.

The term “sequence identity” as used herein refers to a comparison over the entire length of the respective nucleic acid or amino acid sequence to be compared to another, the sequence of interest or subject representing the reference sequence (e.g., in the form of a SEQ ID NO as disclosed herein) wherein these identity or homology values define those as obtained by using the EMBOSS Water Pairwise Sequence Alignments (nucleotide) software (http://www.ebi.ac.uk/Tools/psa/emboss_water/) nucleic acids or the EMBOSS Water Pairwise Sequence Alignments (protein) software (http://www.ebi.ac.uk/Tools/psa/emboss_water/) for amino acid sequences. Those tools provided by the European Molecular Biology Laboratory (EMBL) European Bioinformatics Institute (EBI) for local sequence alignments use a modified Smith-Waterman algorithm (see http://www.ebi.ac.uk/Tools/psa/ and Smith, T. F. & Waterman, M. S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5.

The terms “transformation” and “transfection” are used interchangeably herein for any kind of introduction of a material, in particular a nucleic acid (DNA/RNA) and/or amino acid material, into at least one cell of interest by any kind of physical (e.g., bombardment), chemical or biological (e.g., Agrobacterium) way of introducing the relevant at least one material.

The term “quantitative trait locus” or “QTL” refers to a region of genomic DNA that is associated with and/or influences a quantitative phenotypic trait in at least one genetic background, e.g., in at least one breeding population. A “QTL allele” refers to an allele, whose physical location within the genome is within, including the two outermost (i.e. flanking) positions, the QTL.

### BRIEF DESCRIPTION OF FIGURES

FIG. 1 shows relative quantification of the expression of ZmNdhM, normalized to the housekeeping gene MEP with KE0482 expression set to 1. Significance of difference in means was tested by a two-sided t-test with significance level p<0.01 (n=5 (KE0482), n=5 (KE0678)).

FIG. 2 shows RNAseq reads of inbred lines A188, F7 and six Kemater and six Petkuser DH lines mapped against candidate gene ZmNdhM (Zm00001d002815, B73AGPv4) in control and cold stress conditions. In the yellow-patch line KE0007 expression of ZmNdhM is strongly reduced compared to other DH (double haploid) lines and the reference inbred lines. The landrace DH lines are grouped in cold sensitive and cold tolerant lines based on their performance in phenotyping assays in controlled conditions.

FIG. 3A and B shows plant height and Fv/Fm of heterogeneous inbred families and their parents in control conditions and after cold treatment. The 48-hour time period of the severe cold treatment is indicated in the plot. Significances of effects based on ANOVAs for allele, treatment and allele-treatment interaction are indicated with ns (non-significant), * (p<0.05),**(p<0.01) and *** (p<0.001). FIG. 3A: Plant height: In each biological group (combination of allele and treatment), the mean of plant height for all replicates of HIF1, HIF2 and the parent matching the allele and the associated standard error is shown. KE0678 Allele—cold: n=17; KE0678 Allele—control: n=17; KE0482 Allele—cold: n=19; KE0482 Allele—control: n=19). FIG. 3B: Fv/Fm of heterogeneous inbred families and their parents in control conditions and after cold treatment.

FIG. 4 shows pairwise sequence alignment of the target region (spanning from SEQ ID NO: 4 to SEQ ID NO: 5) derived from PacBio HiFi assemblies of KE0482 and KE0678. The diagonal line represents stretches of DNA sequence with more than 99% sequence identity and a length of more than 1 kb. Dots in the line represent breakpoints in the pairwise alignment. The bars on the x and y axis represent the location of blast hits of B73_AGPv05 genes in the genomes of KE0482 and KE0678.

FIG. 5 shows quantification of the expression of ZmNdhM on protein level by proteomics with TMT labeling. Significance of difference in means between ZmNdhM was tested by a two-sided t-test across genotypes with a significance level of p<0.01 (n=3 (KE0482), n=3 (KE0678), n=3 (HIF1A), n=3 (HIF1B), n=3 (HIF3A), n=3 (HIF3B).

FIG. 6A, B, C and D shows a transposon insertion line and its wild type with expression levels of ZmNdhM normalized to MEP, Fv/Fm and plant height in optimal conditions. Significance of difference in means was tested by a two-sided t-tests with ns (non-significant), * (p<0.05), ** (p<0.01) and *** (p<0.001). FIG. 6A: shows a scheme of the position of the insertion in the 5′UTR of ZmNdhM in the transposon insertion line FIG. 6B: shows relative quantification of the expression of ZmNdhM, normalized to the housekeeping gene MEP with KE0482 expression set to 1. FIG. 6C Plant height: For each genotype the mean of plant height for all replicates and the associated standard error is shown. FIG. 6D: Fv/Fm: For each genotype the mean of plant height for all replicates and the associated standard error is shown.

FIG. 7A and B shows RNA expression levels of ZmNdhM normalized to MEP for 27 Kemater DH lines with or without an insertion in ZmNdhM and the correlation of Fv/Fm with ZmNdhM expression levels. Significance of difference in means was tested by a two-sided t-tests with *** (p<0.001). FIG. 7A: shows relative quantification of the expression of ZmNdhM, normalized to the housekeeping gene MEP with KE0482 expression set to 1. FIG. 7B shows the correlation between Fv/Fm and relative expression of ZmNdhM.

### LIST OF SEQUENCES

SEQ ID NO: 1: KASP marker AX-91512997 of Table 1, wherein y at position 36 is T for the KE0482 “A” allele or C for the KE0678 “B” allele.

SEQ ID NO: 2: KASP marker AX-91513237 of Table 1, wherein m at position 29 is C for the KE0482 “A” allele or A for the KE0678 “B” allele.

SEQ ID NO: 3: KASP marker AX-90736227 of Table 1, wherein k at position 36 is G for the KE0482 “A” allele T for the KE0678 “B” allele.

SEQ ID NO: 4: KASP marker AX-90736268 of Table 1, wherein r at position 33 is A for the KE0482 “A” allele G for the KE0678 “B” allele.

SEQ ID NO: 5: KASP marker AX-90736476 of Table 1, wherein k at position 36 is G for the KE0482 “A” allele T for the KE0678 “B” allele.

SEQ ID NO: 6: KASP marker AX-90736551 of Table 1, wherein y at position 31 is C for the KE0482 “A” allele T for the KE0678 “B” allele.

SEQ ID NO: 7: KASP marker AX-90737128 of Table 1, wherein w at position 36 is T for the KE0482 “A” allele A for the KE0678 “B” allele.

SEQ ID NO: 8: KASP marker AX-90737994 of Table 1, wherein k at position 36 is T for the KE0482 “A” allele G for the KE0678 “B” allele.

SEQ ID NO: 9: KASP primer for AX-91512997 marker, KE0482 “A” allele, forward

SEQ ID NO: 10: KASP primer for AX-91513237 marker, KE0482 “A” allele, forward

SEQ ID NO: 11: KASP primer for AX-90736227 marker, KE0482 “A” allele, forward

SEQ ID NO: 12: KASP primer for AX-90736268 marker, KE0482 “A” allele, reverse

SEQ ID NO: 13: KASP primer for AX-90736476 marker, KE0482 “A” allele, forward

SEQ ID NO: 14: KASP primer for AX-90736551 marker, KE0482 “A” allele, reverse

SEQ ID NO: 15: KASP primer for AX-90737128 marker, KE0482 “A” allele, forward

SEQ ID NO: 16: KASP primer for AX-90737994 marker, KE0482 “A” allele, reverse

SEQ ID NO: 17: KASP primer for AX-91512997 marker, KE0678 “B” allele, forward

SEQ ID NO: 18: KASP primer for AX-91513237 marker, KE0678 “B” allele, forward

SEQ ID NO: 19: KASP primer for AX-90736227 marker, KE0678 “B” allele, forward

SEQ ID NO: 20: KASP primer for AX-90736268 marker, KE0678 “B” allele, reverse

SEQ ID NO: 21: KASP primer for AX-90736476 marker, KE0678 “B” allele, forward

SEQ ID NO: 22: KASP primer for AX-90736551 marker, KE0678 “B” allele, reverse

SEQ ID NO: 23: KASP primer for AX-90737128 marker, KE0678 “B” allele, forward

SEQ ID NO: 24: KASP primer for AX-90737994 marker, KE0678 “B” allele, reverse

SEQ ID NO: 25: KASP primer for AX-91512997 marker, common primer, reverse

SEQ ID NO: 26: KASP primer for AX-91513237 marker, common primer, reverse

SEQ ID NO: 27: KASP primer for AX-90736227 marker, common primer, reverse

SEQ ID NO: 28: KASP primer for AX-90736268 marker, common primer, forward

SEQ ID NO: 29: KASP primer for AX-90736476 marker, common primer, reverse

SEQ ID NO: 30: KASP primer for AX-90736551 marker, common primer, forward

SEQ ID NO: 31: KASP primer for AX-90737128 marker, common primer, reverse

SEQ ID NO: 32: KASP primer for AX-90737994 marker, common primer, forward

SEQ ID NO: 33: second copy of ZmNdhM (Zm00001eb075370) of KE0678, genomic sequence

SEQ ID NO: 34: second copy of ZmNdhM (Zm00001eb075370) of KE0678, coding sequence

SEQ ID NO: 35: second copy of ZmNdhM (Zm00001eb075370) of KE0678, protein sequence

SEQ ID NO: 36: first copy of ZmNdhM (Zm00001eb075370) of KE0678, genomic sequence

SEQ ID NO: 37: first copy of ZmNdhM (Zm00001eb075370) of KE0678, coding sequence

SEQ ID NO: 38: first copy of ZmNdhM (Zm00001eb075370) of KE0678, protein sequence

SEQ ID NO: 39: second copy of ZmNdhM (Zm00001eb075370) of KE0482, genomic sequence

SEQ ID NO: 40: second copy of ZmNdhM (Zm00001eb075370) of KE0482, coding sequence

SEQ ID NO: 41: second copy of ZmNdhM (Zm00001eb075370) of KE0482, protein sequence

SEQ ID NO: 42: first copy of ZmNdhM (Zm00001eb075370) of KE0482, genomic sequence

SEQ ID NO: 43: first copy of ZmNdhM (Zm00001eb075370) of KE0482, coding sequence

SEQ ID NO: 44: first copy of ZmNdhM (Zm00001eb075370) of KE0482, protein sequence

SEQ ID NO: 45: ZmNdhM (Zm00001eb075370) of B73, genomic sequence

SEQ ID NO: 46: ZmNdhM (Zm00001eb075370) of B73, coding sequence

SEQ ID NO: 47: ZmNdhM (Zm00001eb075370) of B73, protein sequence

SEQ ID NO: 48: Zm00001eb075420 of KE0678, genomic sequence

SEQ ID NO: 49: Zm00001eb075420 of KE0678, coding sequence

SEQ ID NO: 50: Zm00001eb075420 of KE0678, protein sequence

SEQ ID NO: 51: Zm00001 eb075420 of KE0482, genomic sequence

SEQ ID NO: 52: Zm00001 eb075420 of KE0482, coding sequence

SEQ ID NO: 53: Zm00001eb075420 of KE0482, protein sequence

SEQ ID NO: 54: Zm00001eb075420 of B73, genomic sequence

SEQ ID NO: 55: Zm00001eb075420 of B73, coding sequence

SEQ ID NO: 56: Zm00001eb075420 of B73, protein sequence

SEQ ID NO: 57: Zm00001 eb075450 of KE0678, genomic sequence

SEQ ID NO: 58: Zm00001 eb075450 of KE0678, coding sequence

SEQ ID NO: 59: Zm00001eb075450 of KE0678, protein sequence

SEQ ID NO: 60: Zm00001 eb075450 of KE0482, genomic sequence

SEQ ID NO: 61: Zm00001 eb075450 of KE0482, coding sequence

SEQ ID NO: 62: Zm00001eb075450 of KE0482, protein sequence

SEQ ID NO: 63: Zm00001eb075450 of B73, genomic sequence

SEQ ID NO: 64: Zm00001eb075450 of B73, coding sequence

SEQ ID NO: 65: Zm00001eb075450 of B73, protein sequence

SEQ ID NO: 66: Zm00001 eb075400 of KE0678, genomic sequence

SEQ ID NO: 67: Zm00001 eb075400 of KE0678, coding sequence

SEQ ID NO: 68: Zm00001eb075400 of KE0678, protein sequence

SEQ ID NO: 69: Zm00001 eb075400 of KE0482, genomic sequence

SEQ ID NO: 70: Zm00001eb075400 of KE0482, coding sequence

SEQ ID NO: 71: Zm00001eb075400 of KE0482, protein sequence

SEQ ID NO: 72: Zm00001eb075400 of B73, genomic sequence

SEQ ID NO: 73: Zm00001eb075400 of B73, coding sequence

SEQ ID NO: 74: Zm00001eb075400 of B73, protein sequence

SEQ ID NO: 75: ZmNdhM (Zm00041ab076840) of Tx303, genomic sequence

SEQ ID NO: 76: ZmNdhM (Zm00041ab076840) of Tx303, coding sequence

SEQ ID NO: 77: ZmNdhM (Zm00041ab076840) of Tx303, protein sequence

SEQ ID NO: 78: ZmNdhM (Zm00041ab076830) of Tx303, genomic sequence

SEQ ID NO: 79: ZmNdhM (Zm00041ab076830) of Tx303, coding sequence

SEQ ID NO: 80: ZmNdhM (Zm00041ab076830) of Tx303, protein sequence

SEQ ID NO: 81: qPCR primer, ZmNdhM (Zm00001 eb075370) forward

SEQ ID NO: 82: qPCR primer, ZmNdhM (Zm00001eb075370) reverse

SEQ ID NO: 83: qPCR primer, MEP (membrane protein PB1A10.07c, Zm00001eb257640) forward

SEQ ID NO: 84: qPCR primer, MEP (membrane protein PB1A10.07c, Zm00001eb257640) reverse

SEQ ID NO: 85: KE0482 sequence corresponding to SEQ ID NO: 86 without deletion

SEQ ID NO: 86: KE0678 1 bp deletion in regulatory region of Zm00001eb075420

SEQ ID NO: 87: B73 sequence corresponding to SEQ ID NO: 86

SEQ ID NO: 88: ZmNdhM Exon2 forward Primer

SEQ ID NO: 89: ZmNdhM_5UTR_reverse Primer

SEQ ID NO: 90: F7_MuMutant CONTIG (Zm00001eb075370)

SEQ ID NO: 91 EF70519264_EF70519264 reverse complement aligning to SEQ ID NO: 90

SEQ ID NO: 92: EF70519268_EF70519268 aligning to SEQ ID NO: 90

### DETAILED DESCRIPTION

The present inventors succeeded in defining a pleiotropic QTL influencing abiotic stress tolerance, including chilling tolerance, early development and the photosynthesis related trait quantum efficiency. The pleiotropic QTL was validated in field experiments and controlled conditions in a bi-parental mapping population. In a bi-parental population derived from two doubled-haploid lines from the landrace “Kemater Landmais Gelb” the QTL could be fine-mapped to a genomic fragment of 676 kB (B73_AGPv05), enclosing 20 genes on chromosome 2 of Zea mays.

Phenotyping of contrasting recombinants showed that growth rates differ both in cold conditions as well as control conditions, but the cold treatment has a stronger adverse effect in recombinants carrying the “B” allele as defined in Table 1. The same pattern was observed for the photosynthesis trait Fv/Fm, showing that the QTL has a significant effect on cold tolerance in recombinants. Reduction of Fv/Fm hints to the cold sensitivity of photosystem 2 as a physiological explanation for the observed differences in the early development of recombinants. In conclusion, the inventors have identified a major locus influencing photosynthesis and early development, in particular under cold conditions.

This sets the basis for using this QTL for breeding purposes without the risk of undesired linkage drag in view of the concise region of the locus of interest. Additionally, a role of the gene ZmNdhM (Zm00001eb075370) was identified, and functional validation studies were conducted. Finally, molecular marker data integration and application allows detection of positive and negative haplotypes at the locus and gene level, characterizing material, and monitoring diversity at and surrounding the locus as such.

In addition, markers are disclosed that allow determining and/or detecting the genomic state at this locus or parts thereof. This information can be linked to phenotype values of parental lines; near isogenic lines (NILs); recombinants and generated recombinants to use the disclosed marker/trait correlations. Altogether, the findings can be integrated into selection processes to select for specific allele compositions and to characterize germplasm in regard to the desired traits as disclosed herein. In particular, the present invention can e.g. be used for “Marker assisted selection” (MAS) of plants having improved traits.

Correspondingly, using single allele state information as well as binned information resulting in haplotypes is the basis for a fast, precise, and improved classification of genetic material during a common selection process. Finally, allelic variation at the ZmNdhM gene level can be used to improve the above-mentioned phenotypes by either modulating expression of the ZmNdhM genes, modifying the molecular activity of such genes and gene products or generating any allelic versions derived from such genes.

In a first aspect, there is provided a method for identifying and/or selecting a plant of the genus Zea having an improved early vigor and/or early plant height phenotype, preferably due to an improved abiotic stress tolerance, including chilling tolerance, and/or an improved quantum efficiency, the method comprising a) providing a plant or a plant population of the genus Zea comprising genomic DNA; and b1) detecting the presence or absence of one, two, three or more QTL allele(s) comprising at least one allele being selected from a “G” at position 23261992, and/or an “A” at position 23478077, and/or a “G” at position 24154201, wherein the positions refer to chromosome 2 and are defined according to the Zea mays physical map B73_AGPv05 reference annotation, preferably wherein the one, two, three or more QTL allele(s) is/are associated with an improved early vigor and/or early plant height phenotype; and/or b2) detecting the presence or absence of at least one functional copy of Zm00001eb075370, and/or a functional fragment, ortholog, homolog or paralog thereof, preferably wherein said a functional fragment, ortholog, homolog or paralog encodes a polypeptide according to any one of SEQ ID NOs: 35, 38, 41, 44, 47, 77 or 80, or a sequence having at least 90%, 91%, 92%, 93%, 94%, 95%, 96%, 97%, 98% or at least 99% sequence identity thereto and/or wherein expression of said gene encoding at least one functional copy of Zm00001eb075370 is about 50% to about 150%, more preferably about 80% to about 120% or about the same level as Zm00001eb075370 expression as in Kemater DH line KE0482 deposited as NCIMB 44328; and c) optionally: selecting at least one plant as having an improved early vigor and/or early plant height phenotype if the one or more of the QTL alleles of b1) and/or the at least one functional copy of Zm00001eb075370 of b2) is/are present.

The method of the first aspect can, for instance, be used as part of or in combination with breeding programs, genome editing, mutagenesis, such as TILLING (Targeting Induced Local Lesions in Genomes) approaches, or other means of developing of new plant lines, including donor lines, comprising the resistance alleles according to the present invention.

An allele can be detected by any means known in the art, including hybridization to allele-specific oligonucleotides, such as competitive allele specific PCR (KASP), or simply by nucleotide sequencing of the respective polymorphic region, including next generation sequencing and/or high throughput sequencing methods.

In certain embodiments, the method may comprise an amplification step, such as PCR amplification or LCR (ligase chain reaction, a technique well known to the skilled person) amplification, to genotype one or more polymorphic positions and/one to detect one or more alleles associated with an improved trait DNA amplifications methods are well known in the art.

Embodiments relating to allele-specific amplification, including KASP, may comprise a non-allele-specific amplification step followed by allele-specific amplification or may use allele-specific amplification direction on the provided genomic DNA.

The method according to the present invention may be utilized for marker-assisted selection (MAS; Sun et al., 2020), including genomic selection (GS) also referred to as genome wide selection (GWS).

In certain embodiments, a donor plant or donor plant population, comprising one or more alleles associated with an improved trait according to the present invention, may be crossed with a recipient plant or a recipient plant population, such as a plant of an elite line or any plant of interest, to introduce one or more alleles associated with an improved trait in the recipient plant or plant population, e.g. as part of a breeding program. The method according to the present invention may be used to identify one or more progenies of such crossings, having an improved trait.

In certain embodiments, one or more selected plants are used for one or more further breeding/crossing steps, including backcrossing, the one or more progenies of said one or more further crossing steps may be selected again for an improved trait by methods according to the invention. Cycles of crossing/breeding and selection of plants having an improved trait according to the present invention may be repeated multiple times.

The term “crossed” or “cross” refers to a sexual cross and involves the fusion of two haploid gametes via pollination to produce diploid progeny (e.g., cells, seeds or plants). The term encompasses both the pollination of one plant by another and selfing (or self-pollination, e.g., when the pollen and ovule are from the same plant).

“Backcrossing” refers to the process by which progeny are repeatedly crossed back to one of the parents, such as the (donor) parent comprising one or more alleles associated with an improved trait according to the present invention. In a backcrossing scheme, the “donor” parent refers to the parental plant with the desired gene/genes, locus/loci, or specific phenotype to be introgressed. The “recipient” parent (used one or more times) or “recurrent” parent (used two or more times) refers to the parental plant into which the gene or locus is being introgressed.

In certain embodiments, one or more plants having at least one improved trait according to the present disclosure may be identified directly, i.e. one or more parts of the plant, comprising genomic DNA of said one or more plants, is removed and used for the method according to the present invention, thereby identifying the presence of one or more alleles associated with an improved trait in, or representing the situation in, said one or more plants itself.

In certain embodiments, the method may comprise regeneration of an entire plant, preferably a fertile plant, from a plant cell, preferably derived from somatic tissue, embryonic tissue, callus tissue or protoplast. Regeneration may also be somatic embryogenesis, which is an artificial process in which a plant or embryo is derived from a single somatic cell or group of somatic cells. Somatic embryos are formed from plant cells that are not normally involved in the development of embryos, i.e. plant tissue like buds, leaves, shoots etc.

In certain embodiments, the method comprises the step of analyzing and/or verifying the desired trait phenotype of one or more selected plants as disclosed herein and/or by using any suitable method known in the art.

The gene ZmNdhM (ORF name Zm00001eb075370, see MaizeGDB (maize genetics and genomics database), NCBI: LOC100277720, EMBL: ONM15409.1), encodes NAD(P)H-quinone oxidoreductase subunit M, involved in cyclic electron transport in photosynthesis. Experiments underlying the present invention have shown a strong correlation between the ZmNdhM gene sequence as well as the ZmNdhM expression with early plant vigor, early, plant height, chilling tolerance and quantum efficiency.

In one embodiment, the method comprises the detection of the presence or absence of an intron sequence of ZmNdhM that does not comprise an insertion, relative to the intron sequence of ZmNdhM according to positions 672 to 897 of SEQ ID NO: 39, that is larger than 100 bp, 50 bp, 30 bp or 10 bp, wherein at least one plant may optionally be selected as having an improved early vigor and/or early plant height phenotype if said intron sequence is present.

In one embodiment, the method comprises the detection of the presence or absence of an intron sequence of ZmNdhM that does not comprise an insertion relative to SEQ ID NO 39 between the nucleotides according to positions 710 and 718 of SEQ ID NO 39, that is larger than 100 bp, 50 bp, 30 bp or 10 bp, optionally wherein the intron sequence does not comprise an insertion between the nucleotides according to positions 710 and 718 of SEQ ID NO 39, wherein at least one plant may optionally be selected as having an improved early vigor and/or early plant height phenotype if said intron sequence is present.

In another embodiment, the method comprises the detection of the presence or absence of an intron sequence of ZmNdhM that does not comprise an insertion between the nucleotides according to positions 23631857 to 23631867 according to the AGPv05 reference annotation that is larger than 100 bp, 50 bp, 30 bp or 10 bp, optionally wherein the intron sequence does not comprise an insertion between the nucleotides according to positions 23631857 to 23631867 according to the AGPv05 reference annotation, wherein at least one plant may optionally be selected as having an improved early vigor and/or early plant height phenotype if said intron sequence is present.

In another embodiment, the method comprises the detection of the presence or absence of an intron sequence of ZmNdhM according to positions 672 to 897 of SEQ ID NO: 39 or a sequence having least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto, wherein at least one plant may optionally be selected as having an improved early vigor and/or early plant height phenotype if said intron sequence is present.

In another embodiment, the method comprises the detection of the presence or absence of a ZmNdhM sequence according to any one of SEQ ID NOs: 39, or a sequence having least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto, wherein at least one plant may optionally be selected as having an improved early vigor and/or early plant height phenotype if said sequence of ZmNdhM is present.

In another embodiment, the method comprises the detection of the presence or absence of a ZmNdhM sequence according to any one of SEQ ID NOs: 45, 75 or 78, or a sequence having least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto, wherein at least one plant may optionally be selected as having an improved early vigor and/or early plant height phenotype if said sequence of ZmNdhM is present.

In another embodiment, the method comprises the detection of the ZmNdhM expression, wherein at least one plant may optionally be selected as having an improved early vigor and/or early plant height phenotype if the ZmNdhM expression is about 50% to about 150%, preferably about 80% to about 120% or about the same level of Zm00001eb075370 expression as in Kemater DH line KE0482, said line having been deposited under the Budapest Treaty on the International Recognition of the Deposit of Cultivars for the purpose of Patent Procedure under the accession number NCIMB 44328 on 18 Jan. 2024 with the National Collection of Industrial, Food and Marine Bacteria (NCIMB, Aberdeen, Scotland).

In another embodiment, the method comprises the detection of at least one variation or mutation in a regulatory and/or in any other region relevant for and/or indicative of an ZmNdhM expression that is about 50% to about 150%, preferably about 80% to about 120% or about the same level of Zm00001eb075370 expression as in Kemater DH line KE0482.

Detection of ZmNdhM expression may be performed by quantifying the ZmNdhM transcript as disclosed herein (through quantitative PCR and/or RNA seq analysis) or by any other means known in the art for quantifying transcripts and/or RNA. The ZmNdhM expression may also be quantified on polypeptide level by any suitable method known in the art.

In some embodiments of the first aspect, the method comprises, in addition or instead of step b1) and/or b2), b3) detecting the presence or absence of a 1 bp deletion in the stretch of nucleotides at positions 23911737 to 23911741 according to the AGPv05 reference annotation in the regulatory region of Zm00001eb075420, wherein at least one plant may optionally be selected as having an improved early vigor and/or early plant height phenotype if said 1 bp deletion is absent. The deletion is shown in SEQ ID NO: 86, which comprises a 1 bp deletion between positions 55 and 56 (numbers relative to the sequence of SEQ ID NO: 86) relative to SEQ ID NO: 85. The 1 bp deletion is in the highly conserved regulatory region of chloroplastic elongation factor G (Zm00001 eb075420).

In another embodiment, the method comprises the detection of the presence or absence of a 1 bp deletion in the stretch of nucleotides at positions 23911737 to 23911741 according to the AGPv05 reference annotation, and the presence or absence of an “A” at position 23911701 and/or a of a “T” at position 23911732 and/or a of a “T” at position 23911772, wherein the positions are according to the AGPv05 reference annotation, wherein at least one plant may optionally be selected as having an improved early vigor and/or early plant height phenotype if said 1 bp deletion is absent and if “A” at position 23911701 and/or a of a “T” at position 23911732 and/or a of a “T” at position 23911769 is/are present. All positions are shown in SEQ ID NO: 85, which comprises an “A” at position 20, a “T” at positon 51 and five “A” nucleotides (no deletion) at positions 56 to 60 and a “T” at position 88 (numbers relative to the sequence of SEQ ID NO: 85).

In another embodiment, the method comprises the detection of the presence or absence of a sequence according to SEQ ID NO: 85 at positions 23911682 to 23911781 according to the AGPv05 reference annotation, wherein at least one plant may optionally be selected as having an improved early vigor and/or early plant height phenotype if said sequence of ZmNdhM is present.

In some embodiments of the first aspect, the method comprises b2) detecting the presence or absence of at least one functional copy of Zm00001eb075370, and/or a functional fragment, ortholog, homolog or paralog thereof by any one of the embodiments defined above; and b3) detecting the presence or absence of a 1 bp deletion at position 23911737 according to the AGPv05 reference annotation in the regulatory region of Zm00001eb075420 by any one of the embodiments defined above. It may be of particular interest, to detect the sequence of Zm00001eb075370 and the conserved regulatory sequence of Zm00001eb075420 in combination to allow best possible detection of the desired traits according to the invention.

In one embodiment of the first aspect, the method comprises an additional step of obtaining at least one plant, or plant cell, seed, tissue, and/or organ thereof identified as having an improved early vigor and/or plant height phenotype based on the detection step b1) and/or b2), preferably by directly obtaining said at least one plant, plant cell, seed, tissue, and/or organ in step c) of selecting at least one plant as having an improved early vigor and/or early plant height phenotype if the one or more of the QTL alleles of b1) and/or the at least one functional copy of Zm00001eb075370 of b2) is/are present.

In some embodiments of the first aspect, the presence or absence of the one, two, three or more QTL alleles is detected using one, two, three or more markers selected from SEQ ID NO: 1 to SEQ ID NO: 8, or a sequence having at least 95%, at least 96%, at least 97%, at least 98%, or at least 99% sequence identity thereto, preferably using at least one marker selected from SEQ ID NO: 4 and/or 5 or a sequence having at least 95%, at least 96%, at least 97%, at least 98%, or at least 99% sequence identity thereto.

In some embodiments of the first aspect, the method comprises the detection of an “A” at position 23478077 as defined above.

In some embodiments of the first aspect, the method comprises the detection of a “G” at position 24154201 as defined above.

In some embodiments of the first aspect, the method comprises the detection of an “A” at position 23478077 and a “G” at position 24154201 as defined above.

In some embodiments of the first aspect, the plant of the genus Zea is a Zea mays plant.

Embodiments according to the various aspects of the present invention can, in particular, be useful for flint corn (Zea mays var. indurate).

In a second aspect, there is provided a method of producing a plant of the genus Zea having an improved early vigor and/or early plant height phenotype, the method comprising the (a) introduction of, and/or (b) mutagenesis to confer, preferably chemical- or radiation-induced mutagenesis, and/or targeted mutagenesis, at least one functional copy of Zm00001eb075370, and/or a functional ortholog, homolog or paralog thereof, optionally wherein the plant of the genus Zea is a Zea mays plant.

Chemical- or radiation-induced mutagenesis, is well established in the art and may be performed by any known suitable method, such as but not limited to EMS (ethyl methanesulfonate) mutagenesis or UV mutagenesis. As chemical- or radiation-induced mutagenesis is a random mutagenesis approach, expression of ZmNdhM may be measured, as disclosed herein or by any suitable method known in the art. Through measuring the expression, mutated one or more plants having an increased expression of ZmNdhM can be identified and/or selected after chemical- or radiation-induced mutagenesis.

Introduction may be through stable or transient introduction by means of transformation and/or insertion using genome-modification technology. Preferably, the introduction of the at least one functional copy comprised its integrated into the chromosomal plant genome and/or the chloroplast genome. Transformation may be performed by any method known in the art, such as but not limited to agrobacterium-mediated transformation, particle bombardment, PEG-mediated transformation, particle uptake or electroporation. Introduction through genome-modification technology may be performed by any site-specific genome modification technique known in the art using an SDN-3 approach, wherein the introduced sequence may be inserted by replacing at least on endogenous copy of ZmNdhM or may be introduced elsewhere in the genome.

Embodiments using a) introduction and/or b) mutagenesis by genome modification, rely on a genome modification system, wherein a genome modification system refers to any DNA, RNA and/or amino acid sequence introduced into the cell, on a suitable vector and/or coated on a particles and/or directly introduced, wherein the genome modification system causes the modification of the genome of the cell in which it has been introduced, wherein the genome modification system comprises at least one site-directed nuclease, nickase or inactivated variant thereof, and optionally at least one further molecule, such as a guide molecule.

A “site-directed nuclease” herein refers to a nuclease or an active fragment thereof, which is capable of specifically recognizing and cleaving DNA at a certain location, the target sequence. Such nucleases typically produce a double-strand break (DSB), which is then repaired by non-homologous end-joining (NHEJ) or homologous recombination (HR). Site-specific nucleases include meganucleases, homing endonucleases, zinc finger nucleases, transcription activator-like nucleases and CRISPR nucleases, or variants including nickases or nuclease-dead variants thereof.

A “CRISPR nuclease”, as used herein, is a specific form of a site-directed nuclease and refers to any nucleic acid guided nuclease which has been identified in a naturally occurring CRISPR system, which has subsequently been isolated from its natural context, and which preferably has been modified or combined into a recombinant construct of interest to be suitable as tool for targeted genome engineering. Any CRISPR nuclease can be used and optionally reprogrammed or additionally mutated to be suitable for the various embodiments according to the present invention as long as the original wild-type CRISPR nuclease provides for DNA recognition, i.e., binding properties. CRISPR nucleases also comprise mutants or catalytically active fragments or fusions of a naturally occurring CRISPR effector sequences, or the respective sequences encoding the same. A CRISPR nuclease may in particular also refer to a CRISPR nickase or even a nuclease-dead variant of a CRISPR polypeptide having endonucleolytic function in its natural environment. A variety of different CRISPR nucleases/systems and variants thereof are meanwhile known to the skilled person and include, inter alia, CRISPR/Cas systems, including CRISPR/Cas9 systems (EP2771468), CRISPR/Cpf1 systems (EP3009511B1), CRISPR/C2C2 systems, CRISPR/CasX systems, CRISPR/CasY systems, CRISPR/Cmr systems, CRISPR/MAD systems, including, for example, CRISPR/MAD7 systems (WO2018236548A1) and CRISPR/MAD2 systems, CRISPR/CasZ systems and/or any combination, variant, or 30 catalytically active fragment thereof. A nuclease may be a DNAse and/or an RNAse, in particular taking into consideration that certain CRISPR effector nucleases have RNA cleavage activity alone, or in addition to the DNA cleavage activity.

The “guide molecule” or “guide nucleic acid sequence” (usually called and abbreviated as guide RNA, crRNA, crRNA+tracrRNA, gRNA, sgRNA, depending on the corresponding CRISPR system representing a prototypic nucleic acid-guided site-directed nuclease system), which recognizes a target sequence to be cut by the nuclease. The at least one “guide nucleic acid sequence” or “guide molecule” comprises a “scaffold region” and a “target region”. The “scaffold region” is a sequence, to which the nucleic acid guided nuclease binds to form a targetable nuclease complex. The scaffold region may comprise direct repeats, which are recognized and processed by the nucleic acid guided nuclease to provide mature crRNA. A pegRNAs may comprise a further region within the guide molecule, the so-called “primer-binding site”. The “target region” defines the complementarity to the target site, which is intended to be cleaved. A crRNA as used herein may thus be used interchangeably herein with the term guide RNA in case it unifies the effects of meanwhile well-established CRISPR nuclease guide RNA functionalities. Certain CRISPR nucleases, e.g., Cas9, may be used by providing two individual guide nucleic acid sequences in the form of a tracrRNA and a crRNA, which may be provided separately, or linked via covalent or non-covalent bonds/interactions. The guide RNA may also be a pegRNA of a Prime Editing system. The at least one guide molecule may be provided in the form of one coherent molecule, or the sequence encoding the same, or in the form of two individual molecules, e.g., crRNA and tracr RNA, orthe sequences encoding the same.

In all embodiments using an insertion nucleotide sequence to be expressed, e.g. sequence encoding a ZmNdhM gene product, such as but not limited to any one of SEQ ID NOs: 35, 38, 41, 44, 47, 77 or 80 the insertion is performed in a manner that allows functional expression. The inserted sequence may comprise all regulatory sequences necessary for efficient transcription and translation operably linked to the nucleotide sequence to be expressed. Alternatively, the nucleotide sequence to be expressed may be inserted without all regulatory sequences necessary for efficient transcription and translation if the insertion lead to an operably linked position relative to regulatory sequences present in the target genome and thereby the efficient transcription and translation are achieved. The skilled person is well aware of suitable regulatory sequences and the particulars of functional expression on plants of the genus Zea.

For SDN2 or SDN3 genome modification, at least one repair template may be delivered with the at least one genome modification or editing system simultaneously or subsequently with the proviso that it will be active, i.e., present and readily available at the site of a genomic target sequence in the plant cell to be modified together with the at least one further tools of interest.

In embodiments using a genome modification system, at least one site-directed nuclease, optionally at least one guide molecule, and (for SDN2 and SDN3) at least one repair template is introduced as DNA, RNA, polypeptide or a combination thereof, wherein a guide molecule and a repair template may be a single molecule. A CRISPR nuclease may be introduced as a complex, i.e. a ribonucleoprotein (RNP), with at least one cognate guide molecule. For SDN1 and SND2 genome editing, it may be desirable to introduce the at least one site-directed nuclease, optionally, the at least one guide molecule and (for SDN2) the at least one repair template as RNA and/or Polypeptide in order to avoid the introduction of transgenic DNA.

Plant cells for use in a method according to the second aspect, can be part of, or can be derived or isolated from any type of plant meristem, including shoot meristem, root meristem and/or inflorescence meristem, in vitro or in vivo. It is possible to use isolated plant cells as well as plant material, i.e. whole plants or parts of plants containing the plant cells. A part or parts of plants may be attached to or separated from a whole, intact plant.

For important cereal crops (e.g., maize, wheat, rye, oat, barley, sorghum, rice), the most widely used explant for genome engineering is immature zygotic embryo.

In some embodiments of the second aspect, the method comprises increasing the expression of Zm00001eb075370, or a functional fragment, ortholog, homolog or paralog thereof, in at least one plant cell.

In one embodiment, the method comprises mutation and/or introduction of at least one regulatory sequence, such as a promoter and/or a transcriptional enhancer and/or a translational enhancer, to increase expression of Zm00001eb075370, or a functional fragment, ortholog, homolog or paralog thereof.

In one embodiment of the second aspect, the method comprises the introduction of at least one nucleic acid molecule comprising or consisting of a nucleic acid sequence according to SEQ ID NO: 39, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto, or of a functional fragment, ortholog, homolog or paralog thereof, or a nucleic acid molecule encoding any one of SEQ ID NOs: 35, 38, 41, 44, 47, 77 or 80, or a sequence having at least 90%, 91%, 92%, 93%, 94%, 95%, 96%, 97%, 98% or at least 99% sequence identity thereto into at least one plant cell.

In some embodiment, comprising the introduction of at least one nucleic acid molecule encoding any one of SEQ ID NOs: 35, 38, 41, 44, 47, 77 or 80, or a sequence having at least 90%, 91%, 92%, 93%, 94%, 95%, 96%, 97%, 98% or at least 99% sequence identity thereto, the expression may be about 50% to about 150%, preferably about 80% to about 120% or about the same level as Zm00001eb075370 expression in Kemater DH line KE0482. Detection of expression may be performed by quantifying the ZmNdhM transcript as disclosed herein (through quantitative PCR and/or RNA seq analysis) or by any other means known in the art for quantifying transcripts and/or RNA. ZmNdhM expression may also be quantified on polypeptide level by any suitable method known in the art.

In one embodiment of the second aspect, the method comprises the introduction of at least one nucleic acid molecule comprising or consisting of a nucleic acid sequence according to SEQ ID NO: 45, 75 and/or 78, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

In another embodiment of the second aspect, the method comprises targeted mutagenesis of at least one plant cell to confer at least one functional copy of Zm00001eb075370 and/or a functional ortholog, homolog or paralog, optionally wherein the targeted mutagenesis is SDN1 genome editing.

Targeted mutagenesis conferring at least one functional copy of Zm00001eb075370, and/or a functional ortholog, homolog or paralog thereof, may be performed by mutating the coding sequence and/or the intronic sequence of the at least one functional copy of Zm00001eb075370, and/or a functional ortholog, homolog or paralog thereof, and/or by mutating and/or inserting at least one regulatory sequence, such as but not limited to a promoter, a terminator and/or a transcriptional and/or translational enhancer, optionally increasing the expression of the at least one functional copy of Zm00001eb075370, and/or a functional ortholog, homolog or paralog thereof.

In one embodiment, the method comprises mutating the intron sequence of ZmNdhM so that it does not comprise an insertion, relative to the intron sequence of ZmNdhM according to positions 672 to 897 of SEQ ID NO: 39, that is larger than 100 bp, 50 bp, 30 bp or 10 bp.

In one embodiment, the method comprises mutating the intron sequence of ZmNdhM so that it does not comprise an insertion between the nucleotides according to positions 710 and 718 of SEQ ID NO 39 that is larger than 100 bp, 100 bp, 50 bp, 30 bp or 10 bp, optionally wherein the intron sequence does not comprise an insertion between the nucleotides according to positions 710 and 718 of SEQ ID NO 39.

In another embodiment, the method comprises mutating the intron sequence of ZmNdhM so that does not comprise an insertion between the nucleotides according to positions 23631857 to 23631867 according to the AGPv05 reference annotation that is larger than 100 bp, 50 bp, 30 bp or 10 bp, optionally wherein the intron sequence does not comprise an insertion between the nucleotides according to positions 23631857 to 23631867 according to the AGPv05 reference annotation.

In another embodiment, the method comprises mutating the intron sequence of ZmNdhM to a sequence according to positions 672 to 897 of SEQ ID NO: 39 or a sequence having least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

In another embodiment, the method comprises mutating ZmNdhM to a sequence according to SEQ ID NOs: 39, or a sequence having least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

In another embodiment, the method comprises mutating ZmNdhM to a sequence according to any one of SEQ ID NOs: 45, 75 or 78, or a sequence having least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

In some embodiments of the second aspect, the method comprises, in addition or instead of the (a) introduction of, and/or (b) mutagenesis to confer, preferably chemical- or radiation-induced mutagenesis, and/or targeted mutagenesis, at least one functional copy of Zm00001eb075370, and/or a functional ortholog, homolog or paralog thereof, (c) inserting 1 nucleotide, preferably an “A”, to remove a 1 bp deletion at position 23911737 according to the AGPv05 reference annotation. The deletion is shown in SEQ ID NO: 86, which comprises a 1 bp deletion between positions 55 and 56 (numbers relative to the sequence of SEQ ID NO: 86) relative to SEQ ID NO: 85.

In another embodiment, the method comprises inserting 1 nucleotide, preferably an “A”, to remove a 1 bp deletion at position 23911737 according to the AGPv05 reference annotation, mutating a nucleotide at position 23911701 to an “A” and/or a nucleotide at position 23911732 to a “T” and/or a nucleotide at position 23911772 to a “T”, wherein the positions are according to the AGPv05 reference annotation. All positions are shown in SEQ ID NO: 85, which comprises an “A” at position 20, a “T” at positon 51 and “A” (no deletion) at position 56 and a “T” at position 91 (numbers relative to the sequence of SEQ ID NO: 85).

In another embodiment, the method comprises mutating the sequence at positions 23911682 to 23911781 according to the AGPv05 reference annotation to a sequence according to SEQ ID NO: 85.

In some embodiments of the second aspect, the method comprises the (a) introduction of, and/or (b) mutagenesis to confer, preferably chemical- or radiation-induced mutagenesis, and/or targeted mutagenesis, at least one functional copy of Zm00001eb075370, and/or a functional ortholog, homolog or paralog thereof, and (c) inserting 1 nucleotide, preferably an “A”, to remove a 1 bp deletion at position 23911737 according to the AGPv05 reference annotation. It may be of particular interest, to mutate and or introduce the/a sequence of Zm00001eb075370 and mutate the conserved regulatory sequence of Zm00001eb075420 in combination to allow best possible detection of the desired traits according to the invention.

In some embodiments, targeted mutagenesis may be performed by prime editing. In some embodiments, the targeted mutagenesis may be performed by base editing. As for Chemical- or radiation-induced mutagenesis, also for embodiments comprising base editing, expression of ZmNdhM may be measured and one or more plants having an increased expression of ZmNdhM may be identified and/or selected after base editing. In embodiments using base editing, the at least one site-directed nuclease is preferably a nickase or inactivated variant thereof.

In a preferred embodiment, the method comprises that the produced plant is not obtained by an essentially biological process. Instead, said plant is obtained by at least one step of artificial human intervention as such not occurring in nature and influencing the plant cell by modifying and/or introducing a step of technical nature influencing sexually crossing and selecting. Such a step may include a step of genome editing, e.g., to exchange a base or nucleotide of interest, a chemical treatment, e.g. for chromosome doubling an agent or gene or gene product including chromosome elimination, the introduction of an exogenous gene or genetic material into a plant genome (nuclear, mitochondrial or plastid genome) and the like, or any combination thereof.

In another aspect, there is provided a plant produced or producible by a method of the second aspect, wherein the plant is not exclusively obtained by means of an essentially biological process.

In a third aspect, there is provided a transgenic plant, or plant cell, seed, tissue, and/or organ thereof of the genus Zea, optionally a Zea mays plant, or plant cell, seed, tissue, and/or organ thereof, comprising at least one functional copy of Zm00001eb075370 and/or a functional fragment, ortholog, homolog or paralog thereof.

Preferably, the transgenic nucleic acid is integrated into the chromosomal genome and/or the chloroplast genome of the plant, wherein the transgenic nucleic acid sequence may have replaced an endogenous copy of ZmNdhM or parts thereof or may be integrated elsewhere.

In one embodiment, the transgenic plant, or plant cell, seed, tissue, and/or organ thereof comprises an intron sequence of ZmNdhM that does not comprise an insertion between the nucleotides according to positions 710 and 718 of SEQ ID NO 39 that is larger than 100 bp, 50 bp, 30 bp or 10 bp, optionally wherein the intron sequence does not comprise an insertion between the nucleotides according to positions 710 and 718 of SEQ ID NO 39.

In another embodiment, the transgenic plant, or plant cell, seed, tissue, and/or organ thereof comprises an intron sequence of ZmNdhM that does not comprise an insertion between the nucleotides according to positions 23631857 to 23631867 according to the AGPv05 reference annotation that is larger than 100 bp, 50 bp, 30 bp or 10 bp, optionally wherein the intron sequence does not comprise an insertion between the nucleotides according to positions 23631857 to 23631867 according to the AGPv05 reference annotation.

In another embodiment, the transgenic plant, or plant cell, seed, tissue, and/or organ thereof comprises an intron sequence of ZmNdhM according to positions 672 to 897 of SEQ ID NO: 39 or a sequence having least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

In another embodiment, the transgenic plant, or plant cell, seed, tissue, and/or organ thereof comprises a ZmNdhM sequence according to SEQ ID NOs: 39, or a sequence having least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

In another embodiment, the transgenic plant, or plant cell, seed, tissue, and/or organ thereof comprises at least one ZmNdhM sequence according to any one of SEQ ID NOs: 45, 75 and/or 78, or a sequence having least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto.

In some embodiment, the transgenic plant, or plant cell, seed, tissue, and/or organ thereof comprises the at least one nucleic acid molecule encoding any one of SEQ ID NOs: 35, 38, 41, 44, 47, 77 or 80, or a sequence having at least 90%, 91%, 92%, 93%, 94%, 95%, 96%, 97%, 98% or at least 99% sequence identity thereto, wherein expression is about 50% to about 150%, preferably about 80% to about 120% or about the same level as Zm00001eb075370 expression in Kemater DH line KE0482. Detection of expression may be performed by quantifying the ZmNdhM transcript as disclosed herein (through quantitative PCR and/or RNA seq analysis) or by any other means known in the art for quantifying transcripts and/or RNA. ZmNdhM expression may also be quantified on polypeptide level by any suitable method known in the art.

In some embodiments the transgenic plant, or plant cell, seed, tissue, and/or organ thereof comprises at least one transgenic regulatory region or a part thereof, to increase the expression of ZmNdhM.

In a fourth aspect, there is provided a use of one, two or more marker(s) capable of detecting the detecting the presence or absence of one, two, three or more of the QTL alleles as defined in claim 1 for identifying and/or selecting a plant of the genus Zea having an improved early vigor and/or early plant height phenotype, preferably wherein the use comprises a use of a marker capable of detecting the detecting the presence or absence of A″ at position 23478077, and/or a marker capable of detecting “G” at position 24154201, optionally a marker capable of detecting the detecting the presence or absence of A″ at position 23478077 and a of a marker capable of detecting “G” at position 24154201, wherein the positions are as defined in above, preferably wherein the one, two or more marker(s) is/are independently selected from SEQ ID NO: 1 to SEQ ID NO: 8, or a sequence having at least 95%, at least 96%, at least 97%, at least 98%, or at least 99% sequence identity thereto, preferably using at least one marker selected from SEQ ID NO: 4 and/or 5 or a sequence having at least 95%, at least 96%, at least 97%, at least 98%, or at least 99% sequence identity thereto.

In some embodiments of the fourth aspect, the use comprises the use of one, two or three or more markers selected from SEQ ID NO: 1 to SEQ ID NO: 8, or a sequence having at least 95%, at least 96%, at least 97%, at least 98%, or at least 99% sequence identity thereto, preferably using at least one marker selected from SEQ ID NO: 4 and/or 5 or a sequence having at least 95%, at least 96%, at least 97%, at least 98%, or at least 99% sequence identity thereto.

In a fifth aspect, there is provided a use of a QTL on chromosome 2, or at least one part thereof, associated with early vigor and/or plant height, flanked by positions 23261992 and 24154201, preferably flanked by positions 23478077 and 24154201, defined according to the Zea mays physical map B73_AGPv05 reference annotation, and/or a use of a functional copy of Zm00001eb075370 of Zm00001eb075370, and/or a functional ortholog, homolog or paralog thereof, for the identification, selection and/or manufacture of a plant of the genus Zea, including a Zea mays plant, having an improved early vigor and/or early plant height phenotype.

In embodiments of the fifth aspect that relate to a use for the manufacture of a plant, said use comprises that the plant is not obtained by an essentially biological process. Instead, said manufactured plant is obtained by at least one step of artificial human intervention as such not occurring in nature and influencing the plant cell by modifying and/or introducing a step of technical nature influencing sexually crossing and selecting. Such a step may include a step of genome editing, e.g., to exchange a base or nucleotide of interest, a chemical treatment, e.g. for chromosome doubling an agent or gene or gene product including chromosome elimination, the introduction of an exogenous gene or genetic material into a plant genome (nuclear, mitochondrial or plastid genome) and the like, or any combination thereof.

The use according to the fourth or the fifth aspect may comprise marker-assisted selection (MAS) strategies, including genomic selection (GS; Nakaya and Isobe, 2012, Van Vleck et al., 1992 and Heffner et al., 2009) also referred to as genome wide selection (GWS).

In certain embodiments of the fourth or the fifth aspect, the use may relate to a donor plant or donor plant population, comprising one or more alleles associated with an improved trait according to the present disclosure, may be crossed with a recipient plant or a recipient plant population, such as a plant of an elite line or any plant of interest, to introduce one or more alleles associated with an improved trait in the recipient plant, e.g. as part of a breeding program, for the identification of one or more progenies of such crossings, having an improved trait. The use may additionally comprise one or more further breeding/crossing, including backcrossing, the progenies of said further crossing steps may be selected again for an improved trait by methods according to the invention.

In certain embodiments of the fourth or the fifth aspect, the use may relate to the identification of a provided plant or plant population itself, having an improved trait according to the present disclosure, wherein one or more parts of the plant or plant population, comprising genomic DNA of said plant or plant population, is taken and used for detection of presence of one or more alleles associated with an improved trait in or representing the situation in said plant or plant population itself.

The use according to the fourth or the fifth aspect may comprise regeneration of an entire plant, preferably a fertile plant, from a plant cell, preferably derived from somatic tissue, embryonic tissue, callus tissue or protoplast. Regeneration may also be somatic embryogenesis, which is an artificial process in which a plant or embryo is derived from a single somatic cell or group of somatic cells. Somatic embryos are formed from plant cells that are not normally involved in the development of embryos, i.e. plant tissue like buds, leaves, shoots etc.

The present invention will now be further illustrated by the following not limiting Examples.

### EXAMPLES

### Example 1: Analyses of the Target QTL Region on Maize Chromosome 2

### Development of KASP Markers

To identify recombinants derived from the cross of KE0482 to KE0678 for fine mapping, molecular markers were developed. KASP markers positioned in the QTL region on chromosome 2 and polymorphic between the two parental lines were synthesized using the publicly available 600 k Axiom™ Maize Genotyping Array (Unterseer et al., 2014) as resource.

TABLE 1
KASP markers derived from 600K array with marker information (name, positional
coordinates) for the genetic map derived from EP1 × PH207 and the physical map
of B73_AGPv05 as well as the corresponding reference (Ref) SNP allele based
on AGPv05 as well as the alternative (Alt) allele. Parental SNP calls are given
for KE0482 and KE0678. “cM” refers to the classical definition of a
centimorgan wherein one cM is equal to a 1% chance that a trait at one genetic
locus will be separated from a trait at another locus due to crossing over in
a single meiosis (meaning the traits cosegregate 99% of the time).

| | | Genetic Map | | | | | | | |
|---|---|---|---|---|---|---|---|---|---|
| SEQ | | EP1 × PH207 | | | | SNP | | SNP Calls in: | |
| ID | | | Pos | B73_AGPv05 | | (AGPv05) | | KE0482 | KE0678 |
| NO: | MarkerID | Chr | (cM) | Chr | Pos | Ref | Alt | Allele “A” | Allele “B” |
| 1 | AX-91512997 | 2 | 50.33 | 2 | 21534401 | T | C | T | C |
| 2 | AX-91513237 | 2 | 51.16 | 2 | 22726499 | C | A | C | A |
| 3 | AX-90736227 | 2 | 52.53 | 2 | 23261992 | G | T | G | T |
| 4 | AX-90736268 | 2 | 52.53 | 2 | 23478077 | A | G | A | G |
| 5 | AX-90736476 | — | — | 2 | 24154201 | G | T | G | T |
| 6 | AX-90736551 | 2 | 54.45 | 2 | 24540732 | C | T | C | T |
| 7 | AX-90737128 | — | — | 2 | 26476289 | A | T | T | A |
| 8 | AX-90737994 | 2 | 57.75 | 2 | 29456545 | G | T | T | G |

### Development of Heterogeneous Inbred Families

F2 plants originating from the cross of KE0482 and KE0678 were grown in 2019. After leaf tissue sampling, genotyping using KASP markers (Table 1) was carried out. Plants showing recombination in the region between markers Seq1 (21534401 bp) and Seq8 (29456545 bp) were self-pollinated. Resulting recombinants were phenotyped in a field experiment in 2020 in three German locations to validate the QTL in field conditions. Recombinants which were heterozygous for the target segment were self-pollinated three more times and resulting F6 plants genotyped using KASP markers (Table 1). Two individual plants which carried a small heterozygous fragment in the target region were self-pollinated to develop heterogeneous inbred families (HIFs). Resulting offspring was genotyped using KASP markers and the two homozygous classes selected to contrast the QTL effect in near isogenic backgrounds. These HIFs assisted in identifying the causal QTL fragment within the 0.68 Mb target region (Table 2).

### Phenotypic Evaluation

Twelve individual plants per marker allele were selected from heterogeneous inbred families HIF1 and HIF2 and grown together with KE0482 and KE0678 in a growth chamber in optimal conditions (25° C./20° C. [day/night]). Plants were randomly assigned to a control group and a cold stress group which was treated with severe cold stress (6° C./2° C. [day/night] for 48 hours, starting 11 days after sowing. Maximum quantum yield efficiency of photosystem 2 (Fv/Fm) was measured in dark adapted plants before light was turned on in the growth chambers, one day before the treatment and at three timepoints after the treatment. The last fully developed leaf (leaf 3) was clipped in the middle with the portable photosynthesis system LI-6800 (LI-COR Inc., Lincoln, NE, USA) for measuring the minimum fluorescence (F0) in the dark-adapted leaf. A saturating light flash was applied to the leaf to measure maximum fluorescence (Fm). Variable fluorescence (Fv) was calculated by subtracting F0 from Fm and used for calculating Fv/Fm. After the treatment all plants were put together in control conditions in a greenhouse. Plant height was measured at 11 time points before and after the treatment, including growth stages V4 and V6. Recombinants were assigned to binary phenotypic groups based on their similarity to their parents KE0482 and KE0678. Plants that looked similar to KE0678 were scored as yellow-patch (YP), plants similar to KE0482 as wild type (WT). The YP phenotype was characterized by a low vigor, light green color, softer leaves and yellow patches on the leaves.

To estimate the effects of KE0482 and KE0678 alleles and their interaction with the cold treatment we fitted a linear mixed model (Equation 1). In the model Yijklmn is the phenotypic value for trait i (i=Fv/Fm, PH) and timepoint j with allele k (k=WT,YP) and genotype I (I=KE0482, KE0678, HIF1, HIF2) undertreatment m (m=cold, control), located in tray n (n=T1, T2, T3, T4); pij is the intercept for trait i and timepoint j; dl is a dummy variable for genotype I (dl=0 for I=KE0482, KE0678; dl=1 for I=HIF1, HIF2); aijk is the fixed effect of allele k for trait i and timepoint j; gijl is the fixed effect of genotype I for trait i and timepoint j; tijm is the fixed effect of treatment m for trait i and timepoint j; aijktijm is the interaction of allele k and treatment m for trait i and timepoint j; tijmbijn is the interaction of treatment m and tray n for trait i and timepoint j; eijklmn is the residual.

$${Y}_{ijklmn} = {μ}_{ij} + {d}_{l} {a}_{ijk} + {g}_{ijl} + {t}_{ijm} + {d}_{l} {a}_{ijk} {t}_{ijm} + {t}_{ijm} {b}_{ijn} + {e}_{ijklmn} \qquad ( 1 )$$

### Identification of DH Lines Differing in Chilling Tolerance

A subset of 116 DH-lines of “Petkuser Ferdinand Rot” (PE) and 238 DH lines of “Kemater Landmais Gelb” (KE) were chosen for phenotyping experiments under controlled growth conditions. Four to six plants per experiment were grown under three different temperature regimes: a) optimal growth condition/control experiment (25° C. during daylight/20° C. during 8 h night), b) mild stress (18° C. during daylight/12° C. during night), c) severe stress (4° C. during daylight/2° C.). In all experiments, light was applied for 16 hours. In the control and mild stress experiment, the growth conditions were constant for a period of 11 or 21 days respectively. In the severe stress experiment, plants were grown at optimal conditions for 11 days, until the V2 stage (second leaf fully developed) was reached. Then the severe stress conditions were applied for 2 days (started 4 hours after “light on”), followed by a period of recovery at optimal temperature for additional 7 days. During the experiment, the following traits were evaluated: plant height, plant vigor, leaf phenotype (color, wilting, rolling, chlorosis, necrosis), lodging.

At the end of the experiments, the plant fresh matter was determined. Experiments were repeated with lines, which performed better or worse than the average DH lines.

Finally, six lines (KE0007, KE0011, KE0090, KE0190, KE0288, KE0407, PE0048, PE0053, PE0075, PE0076, PE0106, PE0190) of each population and the lines F7 and A188 were chosen for transcriptome analyses. The line KE0007 was identified as cold sensitive line and displays the same yellow-patch phenotype as KE0678. As expected, it carries the significant haplotype of KE0678 in the GWAS.

### Transcriptome Analyses

Plants subjected for RNA isolation were grown under slightly adopted conditions. As the most prevalent expression changes were expected to occur within the first hours after the temperature changes, the severe stress temperatures as well as the recovery temperatures were applied for one day only. When the plants reached the V2 stage (11-12 days in control experiment, 21-23 days in mild stress experiment), the complete above ground material of three plants per line (in a few cases only two plants were collected) was collected and immediately frozen in liquid nitrogen. In the mild stress experiment, a second harvest took place after the one-day recovery at optimal temperatures. In the severe stress experiment, material was collected after the stress and after the recovery. Hence five samples each line were collected. Each growth experiment was performed in three completely independent replicates. Total RNA was isolated with the Qiaprep RNA isolation kit. After quality check (NanoDrop; Qubit; Bioanalyser) of the RNA, library preparation and Illumina sequencing were performed at the BMFZ, University of Dusseldorf.

30-45 million high quality (Phred score>32) 1×150 bp single reads were obtained for each sample. The reads were mapped to the B73AGPv4 version of maize reference (CLC Genomics workbench). Genes with >0.5 RPKM (reads per million per kb) were analyzed for differential expression.

Additionally, RNAseq reads were mapped against de-novo assemblies of KE0482 and KE0678 derived from PacBio HiFi long reads with STAR. Resulting BAM files were visualized in IGV browser to detect structurally different transcripts between the de-novo assemblies comparing the RNAseq data of the YP line KE0007 to all other sequenced Kemater DH lines.

### Proteome Analyses

For proteome measurement HIF1A, HIF1B, HIF3A, HIF3B, KE0482 and KE0678 were grown under optimal conditions and cold treated as described above and the last fully developed leaf sampled directly after the cold stress in frozen nitrogen. Total proteome was extracted and measured following the protocol described in Brajkovic et al. 2023 with Tandem Mass Tag (TMT) labeling of peptides after protein digestion for quantification. Proteins were identified and quantified by MaxQuant (Tyanova et al. 2016) using B73_AGPv4 as a reference followed by rescoring the identified proteins using Prosit (Gessulat et al. 2019). Differential expression was assessed by ANOVA using log 2 transformed TMT intensities as response variable and the ZmNdhM allele as the predictor.

### Example 2: Candidate Gene Extraction and RT-qPCR Analysis

Illumina whole genome sequencing data with a coverage of 50× for KE0482 and KE0678 was aligned to B73_AGPv05 using bwa-mem and variants were called with freebayes (Garrison E, Marth G, 2012). The effects of variants were annotated with Variant Effect Predictor (McLaren et al. 2016). Candidate SNPs and InDels were then visually verified in IGV browser.

Whole genome sequencing data using PACBio HiFi long reads was generated for KE0482 and KE0678 and de-novo assembled using the hifiasm assembler (Cheng et al, 2021). The fine-mapped region was extracted from both genotypes by identifying the positions corresponding to Seq ID NO: 4 and Seq ID NO: 5 probes from Table 1 in the de-novo assemblies. KE0482 and KE0678 target regions were aligned using nucmer (Kurtz et al. 2004) to identify structural variation between both genotypes.

Five replicates of the parents of the mapping population, KE0482 and KE0678, were grown in control conditions in the greenhouse and leaf material of plants in stage V6 was sampled from the middle of leaf 5, omitting the mid rip. Total RNA was extracted by phenol-chloroform extraction and reverse-transcribed to cDNA. For validation of candidate genes RT-qPCRs were performed. ZmNdhM was detected using the primer pair SEQ ID NOs: 81 and 82. For normalization RT-qPCR was performed for the house-keeping gene MEP (membrane protein PB1A10.07c, Zm00001eb257640, primer pair: SEQ ID NO: 83 and 84).

### Example 3: Marker/Phenotype Correlations within the Set of Identified Recombinants

For fine-mapping the QTL, recombinants in the target region were identified in a bi-parental mapping population by genotyping with KASP markers (Table 1). Recombinants were subsequently phenotyped for their early plant height, Fv/Fm and scored as yellow-patch (YP) if they show a similar phenotype as the DH line KE0678 as described above.

Two recombinants (HIF1-58 and HIF2-12) with the YP phenotype of KE0678 and matching Fv/Fm values showed recombination between the Markers of SEQ ID NO: 4 (AX-90736268) and SEQ ID NO: 5 (AX-90736476). Based on these recombinants we conclude that the gene causing the QTL is located in a 676 kB genomic segment between these markers (Table 2).

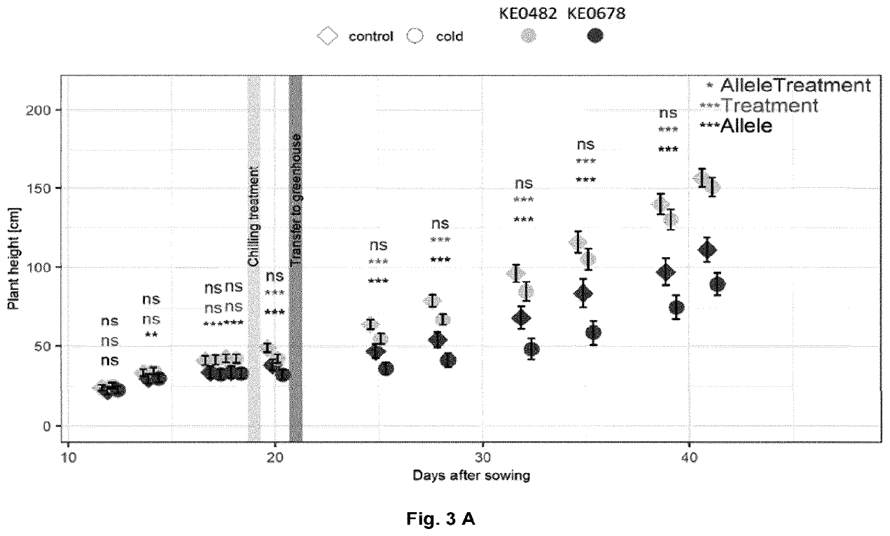

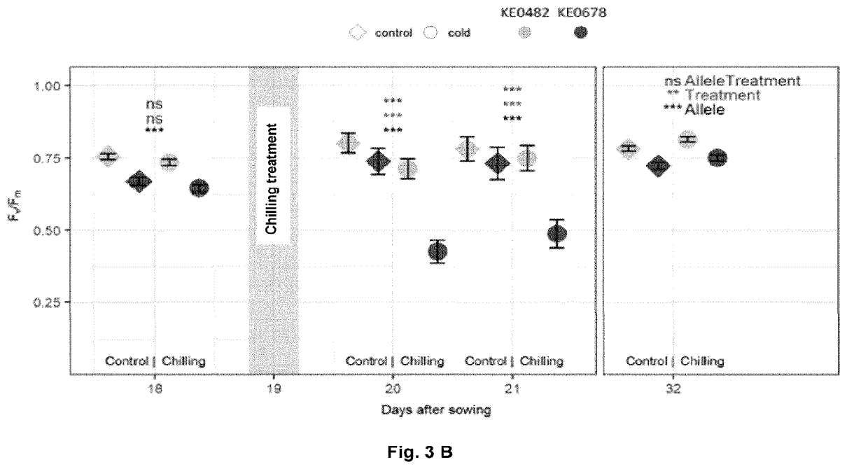

In addition to the yellow-patch phenotype, recombinants carrying the KE0678 allele were significantly smaller at stages V4 and V6 both, in controlled conditions and in a field experiment (table 3, field data not shown). Differing plant heights between QTL alleles were observed in cold treated recombinants as well (table 4). The effect of the allele was significant starting from 14 days after sowing and a significant interaction between the allele and the cold treatment was found starting from 32 days after sowing (FIG. 3A). Interaction of allele and treatment indicate an influence of the QTL on cold sensitivity in the bi-parental mapping population.

TABLE 2
Marker genotypes of parents of the mapping population and
derived heterogeneous inbred families HIF1 and HIF2. The
marker genotype of KE0482 is coded as “A”, the marker
genotype of KE0678 as “B” and heterozygous genotypes
as “H”. HIF1-58 and HIF2-12 are two individual plants
from HIF1 and HIF2 which enabled fine-mapping to the 676
kB genomic fragment between markers Seq4 and Seq7. In the
column “YP” a “+” indicates that the
respective genotype showed the yellow-patch phenotype.

| Genotype | Seq1 | Seq2 | Seq3 | Seq4 | Seq5 | Seq6 | Seq7 | Seq8 | YP |
|---|---|---|---|---|---|---|---|---|---|
| KE0482 | A | A | A | A | A | A | A | A | − |
| KE0678 | B | B | B | B | B | B | B | B | + |
| HIF1_A | A | A | A | A | A | B | B | B | − |
| HIF1_B | A | A | B | B | B | B | B | B | + |
| HIF2_A | A | A | A | A | A | A | A | A | − |
| HIF2_B | A | A | B | B | B | A | A | A | + |
| HIF2-12 | A | A | B | B | H | A | A | A | + |
| HIF1-58 | A | A | H | H | B | B | B | B | + |
| HIF3_A | A | A | A | A | A | A | A | A | − |
| HIF3_B | A | A | B | B | A | A | A | A | + |

Maximum potential quantum efficiency of photosystem II (Fv/Fm) was significantly smaller for plants carrying the KE0678 allele in the target region for all time points and was significantly lower after a cold treatment (Table 3, Table 4, FIG. 3B). The interaction between QTL allele and the treatment was highly significant as well one day and two days after the cold treatment indicating an influence of the QTL on the severity of cold induced damages of Photosystem II (FIG. 3B).

TABLE 3
Means and standard deviation of plant height at stages V4
and V6 (n = 6) and Fv/Fm in control conditions. Recombinants
HIF1-58 and 2-12 were individual plants belonging to groups HIF1_B
and HIF2_B respectively, in this experiment. Their seed numbers will be
increased and the recombinants phenotypically evaluated in the future.

| Genotype | PH_V4 | PH_V6 | Fv/Fm | YP |
|---|---|---|---|---|
| KE0482 | 53.4 ± 3.3 | 99.1 ± 7.4 | 0.76 ± 0.01 | − |
| KE0678 | 45.1 ± 6.3 | 76.3 ± 19.3 | 0.69 ± 0.02 | + |
| HIF1_A | 48.7 ± 6.8 | 95.1 ± 12.2 | 0.77 ± 0.02 | − |
| HIF1_B | 36.9 ± 4.5 | 67.1 ± 12.2 | 0.68 ± 0.03 | + |
| HIF2_A | 40.4 ± 8.7 | 83.5 ± 3.2 | 0.76 ± 0.01 | − |
| HIF2_B | 34.4 ± 5.9 | 68.0 ± 17.4 | 0.70 ± 0.01 | + |
| HIF3_A | 46.8 ± 4.8 | 71.2 ± 5.1 | 0.80 ± 0.01 | − |
| HIF3_B | 35.7 ± 6.4 | 51.5 ± 7.5 | 0.72 ± 0.02 | + |

TABLE 4
Means and standard deviation of plant height at stages V4 and
V6 (n = 6) and Fv/Fm after severe cold treatment. Recombinants
HIF1-58 and 2-12 were individual plants belonging to groups HIF1_B
and HIF2_B respectively, in this experiment. Their seed numbers will be
increased and the recombinants phenotypically evaluated in the future.

| Genotype | PH_V4 | PH_V6 | Fv/Fm | YP |
|---|---|---|---|---|
| KE0482 | 38.5 ± 7.1 | 85.8 ± 20.3 | 0.69 ± 0.02 | − |
| KE0678 | 38.6 ± 6.8 | 54.8 ± 8.2 | 0.43 ± 0.07 | + |
| HIF1_A | 38.7 ± 5.8 | 77.8 ± 18.3 | 0.69 ± 0.08 | − |
| HIF1_B | 31.3 ± 5.4 | 42.1 ± 7.1 | 0.38 ± 0.10 | + |
| HIF2_A | 35.0 ± 8.8 | 70.0 ± 14.1 | 0.69 ± 0.04 | − |
| HIF2_B | 26.3 ± 7.0 | 34.7 ± 13.2 | 0.28 ± 0.16 | + |
| HIF3_A | 52.5 ± 4.9 | 56.2 ± 3.9 | 0.71 ± 0.06 | − |
| HIF3_B | 33.6 ± 5.2 | 37.3 ± 4.9 | 0.55 ± 0.07 | + |

Further Fine-mapping of the region between SEQ ID NO: 4 and SEQ ID NO: 5 is ongoing. The key recombinants are currently being re-sequenced with 50× coverage to identify exact recombination breakpoints in the region between SEQ ID NO: 4 and SEQ ID NO: 5. Moreover, for further mapping of crossings between KE0678 and other Kemater DH lines with more polymorphisms relative to KE0678 in the target region has been initiated to fine-map closer to the causal polymorphism.

### Example 4: Identification and Validation of Candidate Genes

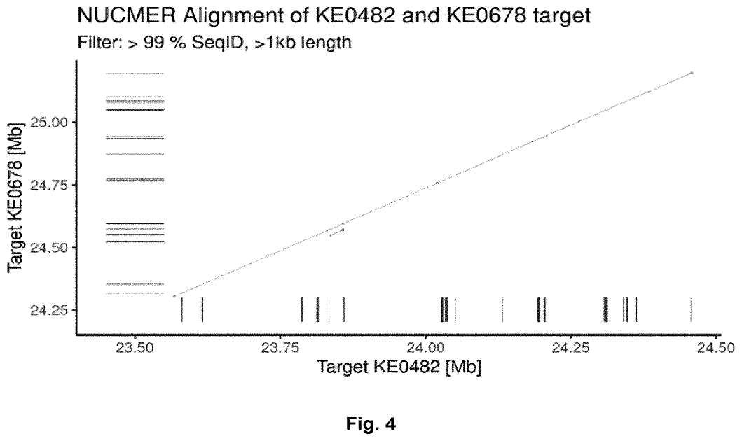

While the QTL encompasses 0.68 Mb in B73_AGPv05, the region between markers of SEQ ID NO: 4 and SEQ ID NO: 5 is about 0.9 Mb in the consensus sequences generated from PACBio reads of KE0482 and KE0678. The sequences of both lines are very similar in this region (FIG. 4). No SNPs overlapping with gene models were identified in the target region between KE0482 and KE0678, indicating expression differences as pivotal for the QTL. Pairwise alignment of the target regions of KE0482 and KE0678 led to the identification of a duplicated genomic region (FIG. 4). In the duplicated region an additional copy of the gene Zm00001eb075370 relative to B73_AGPv05 was located in both assemblies.

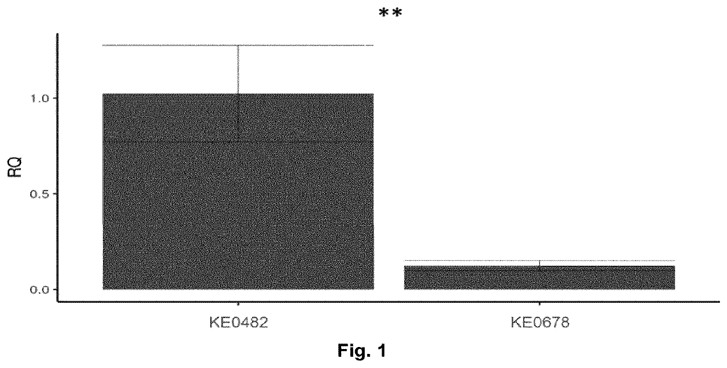

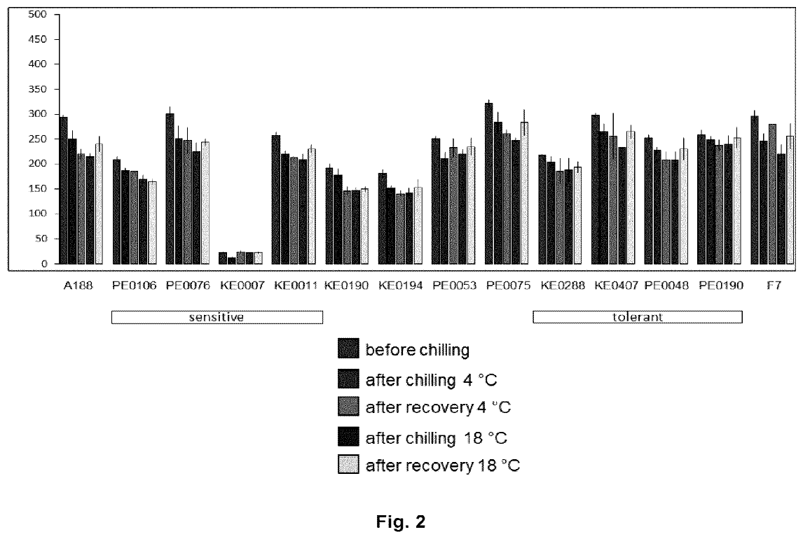

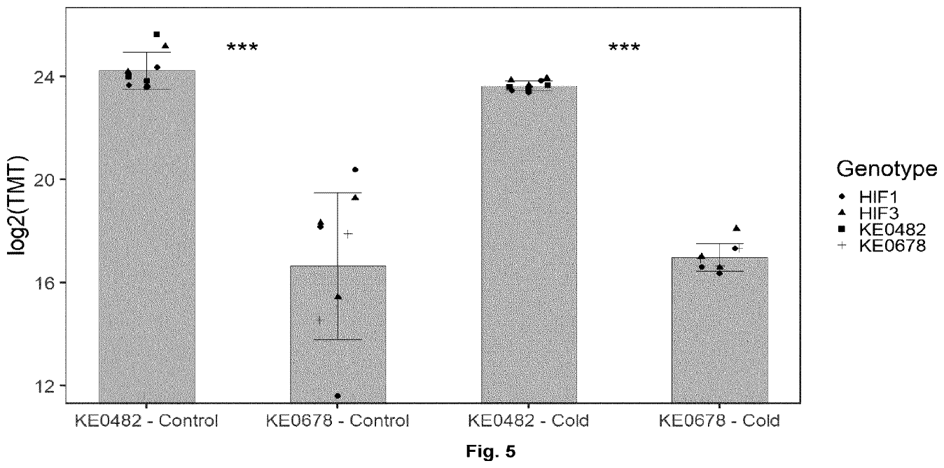

Zm00001eb075370 is annotated as thylakoid located NAD(P)H-quinone oxidoreductase subunit M (NDH-M, ZmNdhM). The first copy is lacking 169 bp coding for the 5′ UTR of the mRNA of the annotated B73 gene. Hence, these copies are presumably not functional in both lines (KE0482 and KE0678). While the second copy of Zm00001eb075370 is intact in KE0482, in KE0678 the second copy is disrupted by an 842 bp insertion within the first intron of the gene. The intron sequence of ZmNdhM is shown as positions 672 to 1739 of SEQ ID NO: 33 for KE0678, with the insertion 842 bp insertion being from position 711 to 1552 of SEQ ID NO: 33, and as positions 672 to 897 of SEQ ID NO 39 for KE0482. It might be that the splicing of the pre-mRNA is impaired by the inserted sequence. Additionally, mapped RNAseq data show that the inserted sequence, which has similarity to an E3-ubiquitin ligase, is transcribed (data not shown). To confirm the hampered expression of Zm00001eb075370, RT-qPCR in optimal conditions at stage V6 with primer pairs binding both copies of the Zm00001eb075370 homologue in both assemblies was conducted. Expression of the candidate ZmNdhM in KE0678 was only about 12% of the expression in KE0482 (FIG. 1). Expression of NDHM was highly different on protein level, only about 1% of the protein levels of lines with the KE0482 allele were identified in lines with the KE0678 allele (FIG. 5). Additionally, Zm00001eb075370 expression was analyzed in a set of six Kemater and six Petkuser DH lines differing in chilling tolerance (FIG. 2). Zm00001eb075370 (ZmNdhM) was the only differentially expressed gene in the QTL. Its expression in the yellow-patch line KE0007 was ten times lower than in the lines without the yellow-patch phenotype, fitting to the observed differences by RT-qPCR. Hence, there is a strong correlation between the observed phenotype and the expression of ZmNdhM. NDH is involved in cyclic electron transport in photosynthesis, and it is known that the cyclic electron transport is involved in stress response. Therefore, ZmNdhM is the most promising candidate gene for being responsible for the observed phenotype. Nevertheless, polymorphisms, located in the target region, may contribute to the observed phenotypes. For example, a one-base deletion in close proximity to Zm00001eb075420 was further identified in K0KE0678 and influence gene expression. Interestingly, KE0482 predominantly showed expression of the longer transcript variant higher Zm00001eb075420_T001, while KE0678 predominantly showed expression of the shorter transcript variant Zm00001eb075420_T002 (cf. Maize Genetics and Genomics Database—MaizeGDB). In the highly conserved regulatory region of chloroplastic elongation factor G (Zm00001eb075420, Table 5) the InDel was identified about 300 bp away from the putative transcription start site. It is well known that regulatory variants can be far away from the genes that they influence in maize. Thus, Zm00001eb075450 (Table 5), annotated as thylakoid-located heat shock protein, is another candidate gene because of its proximity to the InDel. Zm00001eb075400 is a further candidate gene as it is an iron transporter homolog (Table 5) and deficiency in iron homeostasis might explain the observed yellow patches.

TABLE 5
Candidate genes identified for the observed phenotypic differences between
KE0482 and KE0678 within the fine-mapped genomic region. The candidate region
is located on chromosome 2 between SEQ ID NO: 4 and SEQ ID NO: 5.

| SEQ ID | | Position on | | |
|---|---|---|---|---|
| NOs: | ID (B73_AGPv05) | (B73_AGPv05) | Annotation | Notes |
| 33, 36, 39, | Zm00001eb075370 | chromosome 2: | NAD(P)H- | Thylakoid |
| 42, 45 | | 23629471- | quinone | localization, |
| | | 23634057 | oxidoreductase | located in |
| | | | subunit M | duplication |
| 48, 51, 54 | Zm00001eb075420 | chromosome 2: | Elongation | Chloroplast |
| | | 23910599- | factor G, | localization, |
| | | 23917769 | chloroplastic | small |
| | | | | deletion in |
| | | | | highly |
| | | | | conserved |
| | | | | sequence |
| 57, 60, 63 | Zm00001eb075450 | chromosome 2: | HSP70- | Thylakoid |
| | | 23914905- | HSP90 | localization, |
| | | 23927310 | organizing | proximity to |
| | | | protein | small |
| | | | | deletion |
| 66, 69, 72 | Zm00001eb075400 | chromosome 2: | Vacuolar iron | Yellow |
| | | 23728486- | transporter | patches |
| | | 23732583 | homolog 1 | might be |
| | | | | related to |
| | | | | iron |
| | | | | deficiency |

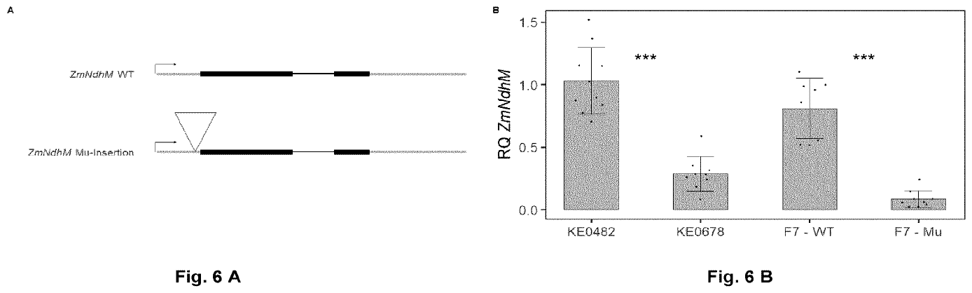

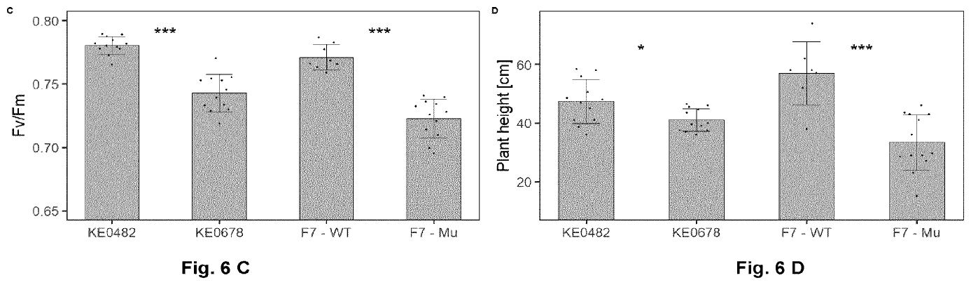

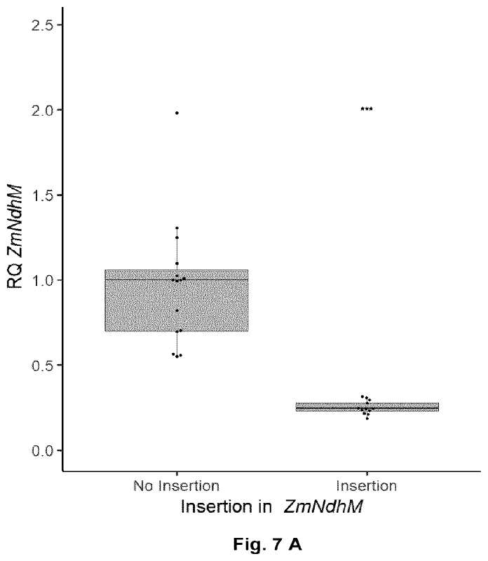

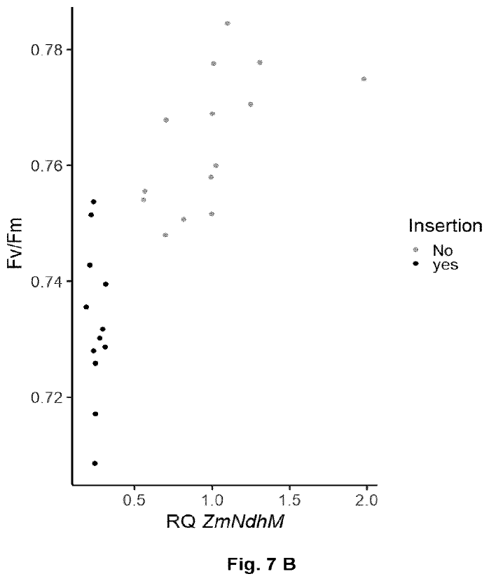

For the candidate gene Zm00001eb075370, Mu-Insertion lines were selected in different backgrounds from the flint heterotic pool (F7, DK105). In F7, a Mutant with an insertion in the 5′UTR of ZmNdhM was validated (FIG. 6 A, cf. SEQ ID NO: 90 for F7_MuMutant contig sequence and SEQ ID NOs: 91 and 92) and phenotyped under optimal conditions. The insertion in the 5′UTR of ZmNdhM reduced its transcript levels (FIG. 6B) and lead to lower Fv/Fm and early plant height (FIG. 6C, FIG. 6D) Additionally, a reverse genetic screen in existing TILLLING populations in PH207 and EP1 background of KWS Saat SE & Co. KGaA is and/or a forward genetic screen by mutagenesis in the Kemater DH line KE0482 to disentangle the function of both gene copies of ZmNdhM by selecting mutations targeting only one copy of the gene is possible. Further, putative causative polymorphisms was evaluated in a broader panel of Kemater DH lines with different alleles of the candidate genes segregating to validate marker—phenotype associations and to assess expression levels of the candidate genes in control and cold conditions. Introducing of KE0678 allele in a cold resistant maize and/or introducing the donor allele of KE0482 into in cold sensitive lines line to validate the association of candidate genes with cold tolerance. 27 Kemater DH lines were grown in the field in 2023 to correlate the previously described insertion in ZmNdhM with RNA levels of ZmNdhM and Fv/Fm. The 842 bp insertion was genotyped in a PCR based assay using SEQ ID 88 and SEQ ID 89 as primers and RT-qPCRs and determination of Fv/Fm conducted as described in previous paragraphs. FIG. 7A shows that RNA levels of ZmNdhM were consistently lower in DH lines with an insertion in ZmNdhM. In FIG. 7B clear differences in levels of Fv/Fm based on the expression of ZmNdhM can be seen. With the help of this assay, the expression level of a functional copy or fragment of Zm00001eb75370 can be determined by determining the transcript level of the RNA. Further detection techniques to quantify transcription/expression levels of RNA, and expression levels of proteins can be used. For example, on RNA level, as a high-throughput alternative to RT-qPCR, gene expression assays can be used, for example using a Quantigene RNA Assay (ThermoFisher). On Protein level, quantification of expression levels can be performed by antibody-based methods, e. g. ELISA (HNASKO, Robert (Hg.). Elisa. New York, NY, USA: Humana Press, 2015.). For Arabidopsis NDHM, an antibody is readily available (https://www.biorbyt.com/ndhm-antibody-orb792644.html).

Further, natural alleles of ZmNdhM could be edited by an SND-1 approach to generate the Donor (KE0482) allele. Another possibility is to exploit natural alleles of ZmNdhM that already resemble the allele of KE0482, for example the American NAM line Tx303 is almost identical to KE0482 in the target region.

For validation of the candidate gene Zm00001eb075420 Mu-Insertion lines with insertions upstream of the 5′UTR of Zm00001eb075420 were identified. Insertions in these mutants were validated by polymerase chain reaction and Sanger sequencing and phenotyping for the target trait Fv/Fm is ongoing.

### REFERENCES

Baker, N. R. and G. Y. Nie (1994). Chilling Sensitivity of Photosynthesis in Maize. Biotechnology in Agriculture—Maize. Y. P. S. Bajaj. Berlin, Heidelberg, Springer Berlin Heidelberg: 465-481.
Bhosale S U, Rymen B, Beemster G T, Melchinger A E, Reif J C. Chilling tolerance of Central European maize lines and their factorial crosses. Ann Bot. 2007 December; 100(6):1315-21. doi: 10.1093/aob/mcm215.
Cheng, H., et al. (2021) Haplotype-resolved de novo assembly using phased assembly graphs with hifiasm. Nat Methods 18, 170-175 (2021). https://doi.org/10.1038/s41592-020-01056-5.
Foyer, C. H., et al. (2002). “Regulation of photosynthesis and antioxidant metabolism in maize leaves at optimal and chilling temperatures: review.” Plant Physiology and Biochemistry 40(6): 659-668.
Frei, O. (2000). “Changes in yield physiology of corn as a result of breeding in northern Europe.” Maydica 45(3): 173-183.
Fryer, M. J., et al. (1998). “Relationship between CO2 Assimilation, Photosynthetic Electron Transport, and Active O2 Metabolism in Leaves of Maize in the Field during Periods of Low Temperature1.” Plant Physiology 116(2): 571-580.
Garrison, E., et al. (2012). “Haplotype-based variant detection from short-read sequencing”. arXiv preprint arXiv:1207.3907 [q-bio.GN] https://doi.org/10.48550/arXiv.1207.3907.
Gessulat et al. 2019—Gessulat, S., Schmidt, T., Zolg, D. P. et al. Prosit: proteome-wide prediction of peptide tandem mass spectra by deep learning. Nat Methods 16, 509-518 (2019). https://doi.org/10.1038/s41592-019-0426-7.
Greaves, J. Improving suboptimal temperature tolerance in maize—the search for variation. Journal of Experimental Botany, 1996; 47(3): 307-323, doi: 10.1093/jxb/47.3.307.
Hashim, H., Al-Shuhaib, M. Exploring the Potential and Limitations of PCR-RFLP and PCR-SSCP for SNP Detection: A Review. Journal of Applied Biotechnology Reports. 2019; 6(4): 137-144. doi: 10.29252/JABR.06.04.02.
Hufford, M B., et al. (2021) “De novo assembly, annotation, and comparative analysis of 26 diverse maize genomes”. Science. 2021 Aug. 6; 373(6555):655-662. doi:10.1126/science.abg5289. PMID: 34353948; PMCID: PMC8733867.
Kucharik, C. J. (2006). “A Multidecadal Trend of Earlier Corn Planting in the Central USA.” Agronomy Journal 98(6): 1544-1550.
Kurtz, S., Phillippy, A., Delcher, A. L. et al. Versatile and open software for comparing large genomes. Genome Biol 5, R12 (2004). https://doi.org/10.1186/gb-2004-5-2-r12.
Leipner, J., et al. (1999). “Effect of growing season on the photosynthetic apparatus and leaf antioxidative defenses in two maize genotypes of different chilling tolerance.” Environmental and Experimental Botany 42(2): 129-139.
Marocco A., Lorenzoni C and Fracheboud Y. Chilling stress on maize. Maydica 2005; (50): 571-580.
Mayer M, Hölker A C, Gonzalez-Segovia E, Bauer E, Presterl T, Ouzunova M, Melchinger A E, Schön C C. Discovery of beneficial haplotypes for complex traits in maize landraces. Nat Commun. 2020 Oct. 2; 11(1):4954. doi: 10.1038/s41467-020-18683-3.
McLaren, W., (2016) et al. The Ensembl Variant Effect Predictor. Genome Biol 17, 122. https://doi.org/10.1186/s13059-016-0974-4.
Poulsen L, Søe M J, Møller L B, Dufva M Investigation of parameters that affect the success rate of microarray-based allele-specific hybridization assays. PLoS One. 2011; 6(3):e14777. doi: 10.1371/journal.pone.0014777.
Revilla, P., et al. (2005). Breeding for Cold Tolerance. Abiotic Stresses, Plant Resistance Through Breeding and Molecular Approaches. P. H. M. Ashraf. New York, Haworth Press: p. 301-398.
Semagn, K., Babu, R., Hearne, S. et al. Single nucleotide polymorphism genotyping using Kompetitive Allele Specific PCR (KASP): overview of the technology and its application in crop improvement. Mol. Breeding. 2014; 33, 1-14. doi:10.1007/s11032-013-9917-x.
Strigens, A., et al. (2012). “Genetic Variation among Inbred Lines and Testcrosses of Maize for Early Growth Parameters and Their Relationship to Final Dry Matter Yield.” Crop Science 52(3): 1084-1092.
Tyanova, Stefka, Tikira Temu, and Juergen Cox. “The MaxQuant computational platform for mass spectrometry-based shotgun proteomics.” Nature protocols 11.12 (2016): 2301-2319.
Unterseer S, Bauer E, Haberer G, Seidel M, Knaak C, Ouzunova M, Meitinger T, Strom T M, Fries R, Pausch H, Bertani C, Davassi A, Mayer K F, Schön C C. A powerful tool for genome analysis in maize: development and evaluation of the high density 600 k SNP genotyping array. BMC Genomics. 2014 Sep. 29; 15(1):823. doi: 10.1186/1471-2164-15-823.
Qu, J., Liu, J. A genome-wide analysis of simple sequence repeats in maize and the development of polymorphism markers from next-generation sequence data. BMC Res. Notes. 2013; 6, 403. doi: 10.1186/1756-0500-6-403.
Zhao X, Tong C, Pang X, Wang Z, Guo Y, Du F, Wu R. Functional mapping of ontogeny in flowering plants. Brief Bioinform. 2012 May; 13(3):317-28. doi: 10.1093/bib/bbr054.

1. A method for identifying and/or selecting a plant of the genus Zea having an improved early vigor and/or early plant height phenotype, the method comprising:
a) providing a plant or a plant population of the genus Zea comprising genomic DNA; and
b1) detecting the presence or absence of one, two, three or more QTL allele(s) comprising at least one allele being selected from the group consisting of a “G” at position 23261992, an “A” at position 23478077, a “G” at position 24154201, and combinations thereof, wherein the positions refer to chromosome 2 and are defined according to a Zea mays physical map B73_AGPv05 reference annotation; and/or
b2) detecting the presence or absence of at least one functional copy of Zm00001eb075370, and/or a functional fragment, ortholog, homolog or paralog thereof; and
c) optionally: selecting at least one plant as having an improved early vigor and/or early plant height phenotype if the one or more of the QTL alleles of b1) and/or the at least one functional copy of Zm00001eb075370 of b2) is/are present.

2. The method of claim 1, wherein the method comprises an additional step of obtaining at least one plant, or plant cell, seed, tissue, and/or organ thereof identified as having an improved early vigor and/or early plant height phenotype based on the detection step b1) and/or b2).

3. The method of claim 1, wherein the presence or absence of the one, two, three or more QTL alleles is detected using one, two, three or more markers selected from the group consisting of SEQ ID NO: 1 to SEQ ID NO: 8, or a sequence having at least 95%, at least 96%, at least 97%, at least 98%, or at least 99% sequence identity thereto.

4. The method of claim 1, wherein the method comprises the detection of an “A” at position 23478077.

5. The method of claim 1, wherein the method comprises the detection of a “G” at position 24154201.

6. The method of claim 1, wherein the method comprises the detection of an “A” at position 23478077 and a “G” at position 24154201.

7. The method of claim 1, wherein the plant of the genus Zea is a Zea mays plant.

8. A method of producing a plant of the genus Zea having an improved early vigor and/or early plant height phenotype, the method comprising the (a) introduction of, and/or (b) mutagenesis to confer at least one functional copy of Zm00001eb075370, and/or a functional ortholog, homolog or paralog thereof, optionally wherein the plant of the genus Zea is a Zea mays plant.

9. The method of claim 8, wherein the method comprises increasing the expression of Zm00001eb075370, or a functional fragment, ortholog, homolog or paralog thereof, in at least one plant cell.

10. The method of claim 8, wherein the method comprises the introduction of at least one nucleic acid molecule comprising or consisting of a nucleic acid sequence according to SEQ ID NO: 39, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity thereto, or of a functional fragment, ortholog, homolog or paralog thereof, or a nucleic acid molecule encoding any one of SEQ ID NOs: 35, 38, 41, 44, 47, 77 or 88, or a sequence having at least 90%, 91%, 92%, 93%, 94%, 95%, 96%, 97%, 98% or at least 99% sequence identity thereto into at least one plant cell.

11. The method of claim 8, wherein the method comprises targeted mutagenesis of at least one plant cell to confer at least one functional copy of Zm00001eb075370 and/or a functional ortholog, homolog or paralog, optionally wherein the targeted mutagenesis is SDN1 genome editing.

12. A transgenic plant, or plant cell, seed, tissue, and/or organ thereof of the genus Zea, optionally a Zea mays plant, or plant cell, seed, tissue, and/or organ thereof, comprising at least one functional copy of Zm00001eb075370 and/or a functional fragment, ortholog, homolog or paralog thereof.

13. A method of using one, two or more marker(s) capable of detecting the presence or absence of one, two, three or more of the QTL alleles of claim 1, the method comprising using the one, two or more marker(s) for identifying and/or selecting a plant of the genus Zea having an improved early vigor and/or early plant height phenotype.

14. The method of claim 13, wherein the one, two or three or more markers is/are selected from the group consisting of SEQ ID NO: 1 to SEQ ID NO: 8, or a sequence having at least 95%, at least 96%, at least 97%, at least 98%, or at least 99% sequence identity thereto.

15. A method of using a QTL on chromosome 2, or at least one part thereof, associated with early vigor and/or plant height, flanked by positions 23261992 and 24154201, wherein the one, two or more marker(s) is/are independently selected from the group consisting of SEQ TD NO: 1 to SEQ TD NO: 8, or a sequence having at least 95%, at least 96%, at least 97%, at least 98%, or at least 99% sequence identity thereto, the method comprising using the QTL for the identification, selection and/or manufacture of a plant of the genus Zea having an improved early vigor and/or early plant height phenotype.

16. The method of claim 1, wherein the improved early vigor and/or early plant height phenotype is due to an improved abiotic stress tolerance, including chilling tolerance, and/or an improved quantum efficiency.

17. The method of claim 1, wherein the one, two, three or more QTL allele(s) is/are associated with an improved early vigor and/or early plant height phenotype.

18. The method of claim 1, wherein said functional fragment, ortholog, homolog or paralog of the at least one functional copy of Zm00001eb075370 encodes a polypeptide according to SEQ ID NO: 35, 38, 41, 44, 47, 77 or 80, or a sequence having at least 90%, 91%, 92%, 93%, 94%, 95%, 96%, 97%, 98% or at least 99% sequence identity thereto and/or wherein expression of said gene encoding the at least one functional copy of Zm00001eb075370 is about 50% to about 150% of a level of Zm00001eb075370 expression as in Kemater DH line KE0482 deposited as NCIMB 44328.

19. The method of claim 1, wherein the method comprises an additional step of directly obtaining at least one plant, or plant cell, seed, tissue, and/or organ thereof in step c).

20. The method of claim 1, wherein the presence or absence of the one, two, three or more QTL alleles is detected using at least one marker selected from the group consisting of SEQ ID NO: 4 and/or 5 or a sequence having at least 95%, at least 96%, at least 97%, at least 98%, or at least 99% sequence identity thereto.
